(19) United States
(12) Patent Application Publication
(10) Pub. No.: US 2008/0171873 A1
(43) Pub. Date: Jul. 17, 2008

(54) Synthesis of selected stereoisomers of certain substituted alcohols

(76) Inventor: Arthur E. Harms, Fairport, NY (US)

Correspondence Address: Bausch & Lomb Incorporated, One Bausch & Lomb Place, Rochester, NY, 14604-2701, US

(21) Appl. No.: 11/652,767

(22) Filed: Jan. 12, 2007

(51) Int. Cl. C07D 209/04; C07D 215/00; C07D 237/00; C07D 307/00; C07D 401/00

(52) U.S. Cl. 544/224; 546/152; 546/276.4; 548/469; 549/469

(57) ABSTRACT

A process for producing one selected stereoisomer of a substituted alcohol comprises reacting a stereoisomeric amine with a halogen-substituted heterocyclic compound, or a stereoisomeric ketone or aldehyde with an amino-substituted heterocyclic compound. The process avoids the production of a racemic mixture of stereoisomers of the prior art. Such a stereoisomeric substituted alcohol can be used for anti-inflammatory therapy.

### BACKGROUND OF THE INVENTION

The present invention relates to the synthesis of selected stereoisomers of certain substituted alcohols. In particular, the present invention relates to the selective synthesis of one stereoisomer of certain substituted alcohols substantially free of another stereoisomer.

The interface between the body and its environment is large, and thus presents many potential opportunities for invasion by environmental virulent pathogens. The outer tissues of the eye constitute parts of this interface, and thus, the eye and its surrounding tissues are also vulnerable to Virulent microorganisms, the invasion and uncontrolled growth of which cause various types of ophthalmic infections, such as blepharitis, conjunctivitis, keratitis, or trachoma, which can result in serious impairment of Vision if untreated. The common types of microorganisms causing ophthalmic infections are Viruses, bacteria, and fungi. These microorganisms may directly invade the surface of the eye, or permeate into the globe of the eye through trauma or surgery, or transmit into the eye through the blood stream or lymphatic system as a consequence of a systemic disease. The microorganisms may attack any part of the eye structure, including the conjunctiva, the cornea, the uvea, the Vitreous body, the retina, and the optic nerve. Ophthalmic infections can cause severe pain, swollen and red tissues in or around the eye, and blurred and decreased Vision.

The body's innate cascade is activated soon after invasion by a foreign pathogen begins. Leukocytes (neutrophils, eosinophils, basophils, monocytes, and macrophages) are attracted to the site of infection in an attempt to eliminate the foreign pathogen through phagocytosis. Leukocytes and some affected tissue cells are activated by the pathogens to synthesize and release proinflammatory cytokines such as IL-1β, IL-3, IL-5, IL-6, IL-8, TNF-α (tumor necrosis factor-α), GM-CSF (granulocyte-macrophage colony-stimulating factor), and MCP-1 (monocyte chemotactic protein-1). These released cytokines then further attract more immune cells to the infected site, amplifying the response of the immune system to defend the host against the foreign pathogen. For example, IL-8 and MCP-1 are potent chemoattractants for, and activators of, neutrophils and monocytes, respectively, while GM-CSF prolongs the survival of these cells and increases their response to other proinflammatory agonists. TNF-α can activate both types of cell and can stimulate further release of IL-8 and MCP-1 from them. IL-1 and TNF-α are potent chemoattractants for T and B lymphocytes, which are activated to produce antibodies against the foreign pathogen.

Although an inflammatory response is essential to clear pathogens from the site of infection, a prolonged or overactive inflammatory response can be damaging to the surrounding tissues. For example, inflammation causes the blood vessels at the infected site to dilate to increase blood flow to the site. As a result, these dilated vessels become leaky. After prolonged inflammation, the leaky vessels can produce serious edema in, and impair the proper functioning of, the surrounding tissues (see; e.g., V. W. M. van Hinsbergh, Arteriosclerosis, Thrombosis, and Vascular Biology, Vol. 17, 1018 (1997)). In addition, a continued dominating presence of macrophages at the injured site continues the production of toxins (such as reactive oxygen species) and matrix-degrading enzymes (such as matrix metalloproteinases) by these cells, which are injurious to both the pathogen and the host's tissues. Therefore, a prolonged or overactive inflammation should be controlled to limit the unintended damages to the body and to hasten the body's recovery process.

Glucocorticoids (also referred to herein as “corticosteroids”) represent one of the most effective clinical treatment for a range of inflammatory conditions, including acute inflammation. However, steroidal drugs can have side effects that threaten the overall health of the patient.

It is known that certain glucocorticoids have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Unlike acute ocular inflammation associated with physical trauma or infection of the outer surface of the anterior portion of the eye, which requires short-term therapy on the order of a few weeks, infection and inflammation of the posterior portion of the eye can require treatment for extended periods of time, generally several months or more. This chronic use of corticosteroids significantly increases the risk of IOP elevations. In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide pharmaceutical compounds and compositions to treat, control, reduce, ameliorate, or prevent inflammation or infections and their inflammatory sequelae, which compounds and compositions cause a lower level of at least an adverse side effect than a composition comprising at least a prior-art glucocorticoid used to treat, reduce, or ameliorate the same conditions. Certain substituted alcohols have been disclosed to have anti-inflammatory properties similar to those of glucocorticoids, but with lower levels of some side effects (see; e.g., U.S. Pat. Nos. 6,897,224 and 7,109,212 and U.S. patent application Publication 2006/0116396). It is often found that one of the stereoisomers of these substituted alcohols has higher efficacy than the other stereoisomer. However, the prior-art syntheses of these substituted alcohols (as disclosed in these patents and patent application) typically yield a racemic mixture, which requires elaborate separation and increases the manufacturing cost. Therefore, it is very desirable to provide a method for producing only the selected stereoisomer of a desired substituted alcohol.

### SUMMARY

In general, the present invention provides a method for selectively producing a stereoisomer of a substituted alcohol that has a Formula Ia or Ib,

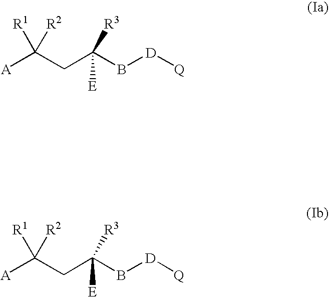

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ cycloalkyl group (or alternatively, C₃-C₆, or C₃-C₅, or C₃ cycloalkyl group); R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; B comprises a methylene or substituted methylene group, wherein a substituent on the methylene group is C₁-C₅ alkyl (or alternatively, C₁-C₃ alkyl), hydroxy, halogen, or amino; E is hydroxy; and D is —NH— or NR′—, wherein R′ comprises an unsubstituted or substituted C₁-C₁₅ (alternatively, C₁-C₁₀, or C₁-C₅, or C₁-C₃) linear or branched alkyl group. The method comprises reacting a compound having Formula IVa or IVb

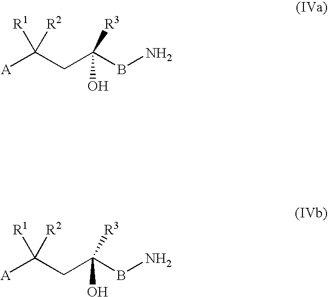

with a compound having a formula of Q-X under a base catalysis condition (such as in the presence of a tertiary amine, alkali carbonate, or alkali hydroxide) or under transition metal catalysis (such as palladium or platinum catalysis), wherein X is a halogen (such as bromine, chlorine, fluorine, or iodine) or the tosylate group. Such a reaction is recognized as the Buchwald-Hartwig reaction (see; e.g., J. P. Wolfe, S. Wagaw, J.-F. Marcoux, and S. L. Buchwald, Acc. Chem. Res., Vol. 31, 805 (1998); J. F. Hartwig, Acc. Chem. Res., Vol. 31, 852 (1998)).

Alternatively, the method comprises reacting a compound having Formula Va or Vb

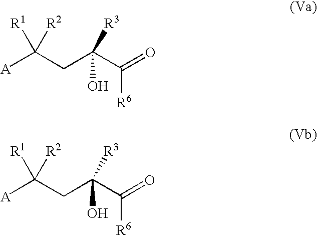

with a compound having a formula of Q-NH₂ (or Q-NHR′), wherein R⁶ is hydrogen, C₁-C₅ alkyl (or alternatively, C₁-C₃ alkyl), halogen, or amino; and R′ has the meaning disclosed above. Such a reaction is recognized as a reductive amination.

In one aspect, a method of the present invention produces a substantially pure isomer having Formula Ia or Ib.

In another aspect of the present invention, compound having Formula IVa or IVb is obtained by a method comprising: (a) converting a chiral epoxyester or epoxycarboxamide having Formula VIa or VIb to a chiral primary amine having Formula VIIa or VIIb; and (b) reducing the chiral primary amine having Formula VIIIa or VIIIb to form the compound having Formula IVa or IVb.

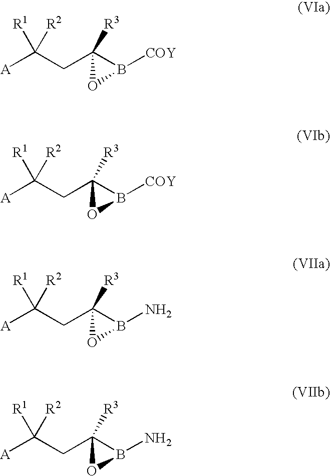

wherein Y is OR*, NHR*, or NH₂, and R* represents a chiral auxiliary.

In still another aspect, a compound having Formula Va or Vb is obtained by a method comprising: (a) converting a chiral carboxamide having Formula VIa or VIb to a chiral primary amine having Formula VIIa or VIIb; and (b) converting the chiral primary amine having Formula VIIa or VIIb under an acidic condition to a chiral aldehyde or ketone having Formula Va or Vb.

Other features and advantages of the present invention will become apparent from the following detailed description and claims.

### DETAILED DESCRIPTION

Glucocorticoids (“GCs”) are among the most potent drugs used for the treatment of allergic and chronic inflammatory diseases or of inflammation resulting from infections. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-1, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol. 94, 557-572 (1998). On the other hand, there is persuasive evidence that the synthesis of IκBα, which are proteins having inhibitory effects on the NF-κB proinflammatory transcription factors, is increased by GCs. These proinflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäcke et al., Pharmacol. Ther., Vol. 96, 23-43 (2002). Thus, while the transactivation of certain genes by GCs produces beneficial effects, the transactivation of other genes by the same GCs can produce undesired side effects, one of which is glaucoma. Therefore, GCs would not be employed to treat or prevent glaucoma or its progression. Consequently, it is very desirable to provide pharmaceutical compounds and compositions that produce differentiated levels of transactivation and transrepression activity on GC-responsive genes such that undesired side effects are not produced or at least are minimized.

In certain aspects, a compound that produces differentiated levels of transactivation and transrepression activity on GC-responsive genes such that undesired side effects are not produced or at least are minimized can satisfy some unmet needs for therapies that heretofore have relied on glucocorticoids. Such a compound, termed herein a dissociated glucocorticoid receptor agonist (“DIGRA”), is capable of binding to the glucocorticoid receptor (which is a polypeptide) and, upon binding, is capable of producing differentiated levels of transrepression and transactivation of gene expression. A compound that binds to a polypeptide is sometimes herein referred to as a ligand.

As used herein, the term “alkyl” or “alkyl group” means a linear- or branched-chain saturated aliphatic hydrocarbon monovalent group, which may be unsubstituted or substituted. The group may be partially or completely substituted with halogen atoms (F, Cl, Br, or I). Non-limiting examples of alkyl groups include methyl, ethyl, n-propyl, 1-methylethyl(isopropyl), n-butyl, n-pentyl, 1,1-dimethylethyl(t-butyl), and the like. It may be abbreviated as “Alk”.

As used herein, the term “alkenyl” or “alkenyl group” means a linear- or branched-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

As used herein, the term “alkynyl” or “alkynyl group” means a linear- or branched-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

As used herein, the term “alkylene” or “alkylene group” means a linear- or branched-chain saturated aliphatic hydrocarbon divalent radical having the specified number of carbon atoms. This term is exemplified by groups such as methylene, ethylene, propylene, n-butylene, and the like, and may alternatively and equivalently be denoted herein as “-(alkyl)-”.

The term “alkenylene” or “alkenylene group’ means a linear- or branched-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as “-(alkylenyl)-”.

The term “alkynylene” or “alkynylene group” means a linear- or branched-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as “-(alkynyl)-′”.

As used herein, the term “aryl” or “aryl group” means an aromatic carbocyclic monovalent or divalent radical of from 5 to 16 carbon atoms having a single ring (e.g., phenyl or phenylene), multiple condensed rings (e.g., naphthyl or anthranyl), or multiple bridged rings (e.g., biphenyl). Unless otherwise specified, the aryl ring may be attached at any suitable carbon atom which results in a stable structure and, if substituted, may be substituted at any suitable carbon atom which results in a stable structure. In some embodiments, the aryl group comprises from 5 to 14 carbon atoms. In some other embodiments, the aryl group comprises from 5 to 10 carbon atoms. Non-limiting examples of aryl groups include phenyl, naphthyl, anthryl, phenanthryl, indanyl, indenyl, biphenyl, and the like. It may be abbreviated as “Ar”.

The term “heteroaryl’ or “heteroaryl group” means a stable aromatic 5- to 16-membered, monocyclic or polycyclic monovalent or divalent radical, which may comprise one or more fused or bridged ring(s), preferably a 5- to 7-membered monocyclic or 7- to 10-membered bicyclic radical, having from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

The term “heterocycle”, “heterocycle group”, “heterocyclyl”, “heterocyclyl group”, “heterocyclic”, or “heterocyclic group” means a stable non-aromatic 5- to 16-membered monocyclic or polycyclic, monovalent or divalent, ring which may comprise one or more fused or bridged ring(s), preferably a 5- to 7-membered monocyclic or 7- to 10-membered bicyclic ring, having from one to three heteroatoms in at least one ring independently selected from nitrogen, oxygen, and sulfur, wherein any sulfur heteroatoms may optionally be oxidized and any nitrogen heteroatom may optionally be oxidized or be quaternized. As used herein, a heterocyclyl group excludes heterocycloalkyl, heterocycloalkenyl, and heterocycloalkynyl groups. Unless otherwise specified, the heterocyclyl ring may be attached at any suitable heteroatom or carbon atom which results in a stable structure and, if substituted, may be substituted at any suitable heteroatom or carbon atom which results in a stable structure. Non-limiting examples of heterocycles include pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl, and the like.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl(tetralin), 1-decalinyl, bicyclo[2.2.2]octanyl, 1-methylcyclopropyl, 2-methylcyclopentyl, 2-methylcyclooctyl, and the like.

The term “cycloalkenyl” or “cycloalkenyl group” means a stable aliphatic 5- to 15-membered monocyclic or polycyclic monovalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenyl ring may be attached at any carbon atom which results in a stable structure and, if substituted, may be substituted at any suitable carbon atom which results in a stable structure. Exemplary cycloalkenyl groups include cyclopentenyl, cyclohexenyl, cycloheptenyl, cyclooctenyl, cyclononenyl, cyclodecenyl, norbornenyl, 2-methylcyclopentenyl, 2-methylcyclooctenyl, and the like.

The term “cycloalkynyl” or “cycloalkynyl group” means a stable aliphatic 8- to 15-membered monocyclic or polycyclic monovalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynyl ring may be attached at any carbon atom which results in a stable structure and, if substituted, may be substituted at any suitable carbon atom which results in a stable structure. Exemplary cycloalkynyl groups include cyclooctynyl, cyclononynyl, cyclodecynyl, 2-methylcyclooctynyl, and the like.

The term “carbocycle” or “carbocyclic group’ means a stable aliphatic 3- to 15-membered monocyclic or polycyclic monovalent or divalent radical consisting solely of carbon and hydrogen atoms which may comprise one or more fused or bridged rings, preferably a 5- to 7-membered monocyclic or 7- to 10-membered bicyclic ring. Unless otherwise specified, the carbocycle may be attached at any carbon atom which results in a stable structure and, if substituted, may be substituted at any suitable carbon atom which results in a stable structure. The term comprises cycloalkyl (including spiro cycloalkyl), cycloalkylene, cycloalkenyl, cycloalkenylene, cycloalkynyl, and cycloalkynylene, and the like.

The terms “heterocycloalkyl”, “heterocycloalkenyl”, and “heterocycloalkynyl” mean cycloalkyl, cycloalkenyl, and cycloalkynyl group, respectively, having at least a heteroatom in at least one ring, respectively.

As used herein, the phrase “substantiall free of the ther steroisomer” means containing less than 5 mole percent of the other stereoisomer; preferably, less than 3, and more preferably, less than 1 mole percent of the other stereoisomer.

In general, the present invention provides a method for selectively producing a stereoisomeric compound having Formula Ia or Ib,

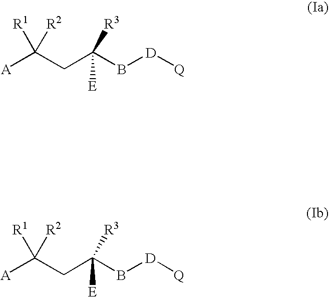

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ (or alternatively C₃-C₆, or C₃-C₅, or C₃) cycloalkyl group; R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; B comprises a methylene or substituted methylene group, wherein a substituent on the methylene group are independently C₁-C₅ alkyl (or alternatively, C₁-C₃ alkyl), hydroxy, halogen, or amino group; E is hydroxy; and D is —NH— or —NR′—, wherein R′ comprises an unsubstituted or substituted C₁-C₁₅ (alternatively, C₁-C₁₀, or C₁-C₅, or C₁-C₃) linear or branched alkyl group. The method comprises reacting a chiral primary amine having Formula IVa or IVb

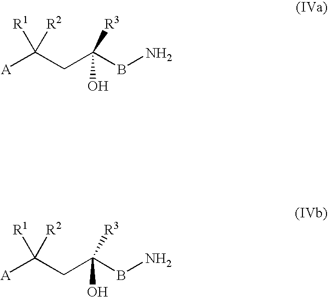

with a compound having a formula of Q-X under a base catalysis condition (such as in the presence of a tertiary amine, alkali carbonate, or alkali hydroxide) or under transition metal catalysis (such as palladium or platinum catalysis), wherein X is a halogen or tosylate group, such as bromine, chlorine, fluorine, or iodine.

Alternatively, the method comprises reacting a compound having Formula Va or Vb

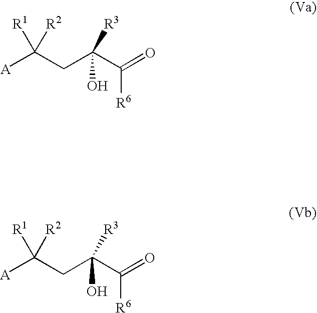

with a compound having a formula of Q-NH₂ (or Q-NHR′), wherein R⁴ is hydrogen, C₁-C₅ alkyl (or alternatively, C₁-C₃ alkyl), hydroxy, halogen, or amino; and R′ has the meaning disclosed above.

In one embodiment, B is the methylene group.

In another embodiment, A and Q are independently selected from the group consisting of aryl and heteroaryl groups substituted with at least a halogen atom, cyano group, hydroxy group, or C₁-C₁₀ alkoxy group (alternatively, C₁-C₅ alkoxy group, or C₁-C₃ alkoxy group); R¹, R², and R³ are independently selected from the group consisting of unsubstituted and substituted C₁-C₅ alkyl groups (preferably, C₁-C₃ alkyl groups); B is a methylene group; D is the —NH— or —NR′— group, wherein R′ is a C₁-C₅ alkyl group (preferably, C₁-C₃ alkyl group); and E is the hydroxy group.

In still another embodiment, A comprises a dihydrobenzofuranyl group substituted with a halogen atom; Q comprises a quinolinyl or isoquinolinyl group substituted with a C₁-C₁₀ alkyl group; R¹ and R² are independently selected from the group consisting of unsubstituted and substituted C₁-C₅ alkyl groups (preferably, C₁-C₃ alkyl groups); B is a methylene group; D is the —NH— group; E is the hydroxy group; and R³ comprises a completely halogenated C₁-C₁₀ alkyl group (preferably, completely halogenated C₁-C₅ alkyl group; more preferably, completely halogenated C₁-C₃ alkyl group).

In yet another embodiment, A comprises a dihydrobenzofuranyl group substituted with a fluorine atom; Q comprises a quinolinyl or isoquinolinyl group substituted with a methyl group; R¹ and R² are independently selected from the group consisting of unsubstituted and substituted C₁-C₅ alkyl groups; B is a methylene group; D is the —NH— group; E is the hydroxy group; and R³ comprises a trifluoromethyl group.

Compounds having Formula Ia or Ib are useful as a dissociated glucocorticoid receptor agonist (“DIGRA”).

In one aspect of the present invention, a selected stereoisomeric compound having Formula Ia or Ib is produced by a method comprising reacting a chiral primary amine having Formula IVa or IVb

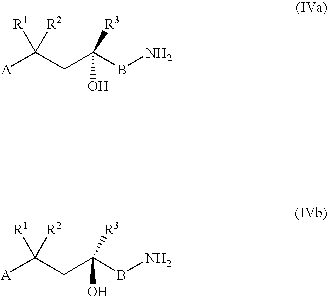

with a compound having a formula of Q-X under a base catalysis condition (such as in the presence of a tertiary amine, alkali carbonate, or alkali hydroxide) or under transition metal catalysis (such as palladium or platinum catalysis), wherein X is a halogen, such as bromine, chlorine, fluorine, or iodine; and A, Q, B, R¹, R², and R³ have the various meanings disclosed herein above. Such a reaction is recognized as the Buchwald-Hartwig reaction.

Alternatively, the method comprises reacting a compound having Formula Va or Vb

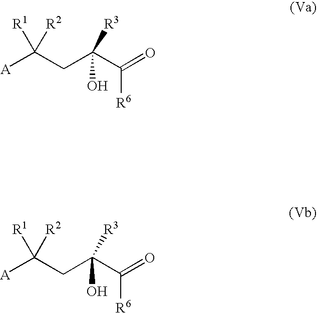

with a compound having a formula of Q-NH₂ (or Q-NHR′), wherein R⁶ is hydrogen, C₁-C₅ alkyl (or alternatively, C₁-C₃ alkyl), hydroxy, halogen, or amino; R′ comprises an unsubstituted or substituted C₁-C₁₅ (alternatively, C₁-C₁₀, or C₁-C₅, or C₁-C₃) linear or branched alkyl group; and R″ is hydrogen or a C₁-C₅ alkyl group (preferably, C₁-C₃ alkyl group); A, Q, R¹, R², and R³ have the various meanings disclosed above. Such a reaction is recognized as a reductive amination.

In one aspect of the present invention, a compound having Formula IVa or IVb can be prepared according a method comprising: (a) converting a chiral epoxyester or epoxycarboxamide having Formula VIa or VIb to a chiral primary epoxyamine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite; preferably, sodium hypobromite or sodium hypochlorite) (see;. e.g., T. Shioiri, Comp. Org. Syn., Vol. 6, 800 (1991)); and (b) reducing the chiral primary epoxyamine having Formula VIIa or VIIb to form the chiral primary amine having Formula IVa or IVb.

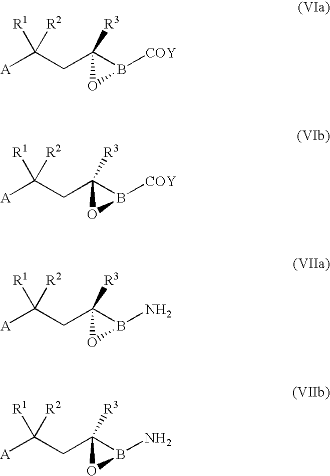

Such a method can be illustrated in Scheme 1.

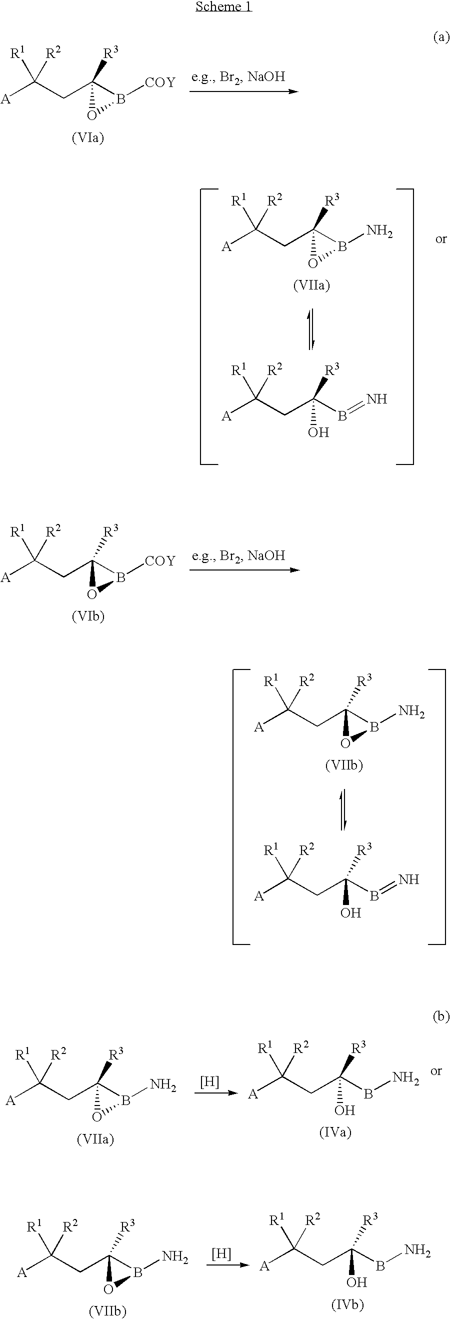

Step (b) of the method disclosed immediately above may be carried out using a reducing agent such as LiAlH₄, NaBH₄, diisobutyl aluminum hydride (“DIBAL”), a 65% (by weight) solution of sodium bis(2-methoxyethoxy)aluminum hydride in toluene, or a mixture of trifluoroacetic acid anhydride and sodium iodide (P. Bravo et al., J. Org. Chem., Vol. 57, 2726 (1992)), a mixture of trifluoroacetic acid anhydride and 2,4,6-trimethylpyridine (P. Bravo et al., J. Org Chem., Vol. 55, 4216 (1990)), or hydrogen chloride in ethanol (J. L. Garcia Ruano et al., J. Org. Chem., Vol. 59, 533 (1994)). Alternatively, this reduction may be carried out under hydrogen in the presence of a transition metal catalyst, such as Pd or Pt catalyst.

In yet another aspect of the invention, when the reduction step (b) is performed with a reducing agent, such as the aluminum hydride reagents listed above, a suitable solvent is diethyl ether, toluene, tetrahydrofuran (“THF”), tert-butyl methyl ether (“MTBE”), hexanes, or a mixture thereof. Otherwise, a suitable solvent for step (b) is diethyl ether, toluene, THF, MTBE, hexanes, benzene, acetonitrile, acetone, dichloromethane, ethyl acetate, or a mixture thereof.

In an alternative embodiment of the present invention, a compound having Formula IVa or IVb can be prepared according a method comprising: (a) converting a chiral epoxyester or epoxycarboxamide having Formula VIa or VIb to a chiral primary epoxyamine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite); and (b) converting the chiral primary epoxyamine having Formula VIIa or VIIb under an acidic condition, for example in an aqueous medium, to the chiral aldehyde or ketone having Formula Va or Vb.

Such a method can be illustrated in Scheme 2.

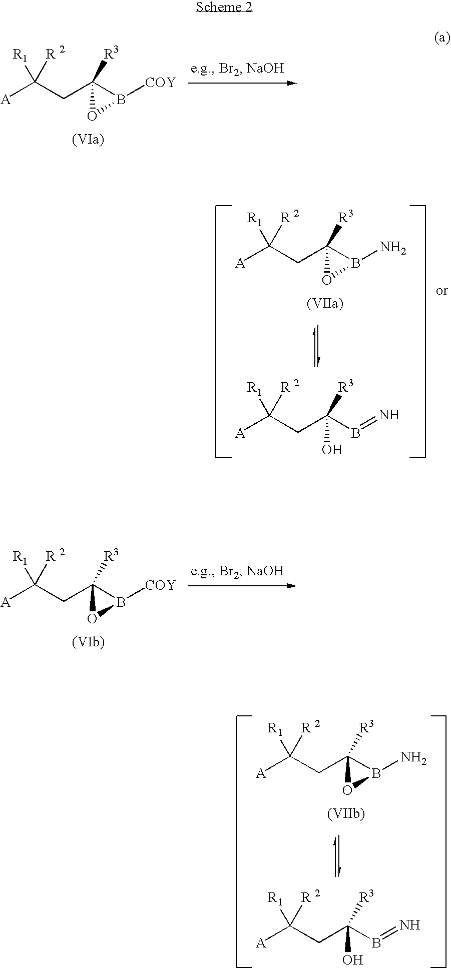

wherein Y is OR*, NH₂, or NHR*, and R* is a chiral auxiliary. When Y is OR*, the compound VIa or VIb is reacted with ammonia or an amine before the Hoffman rearrangement reaction;

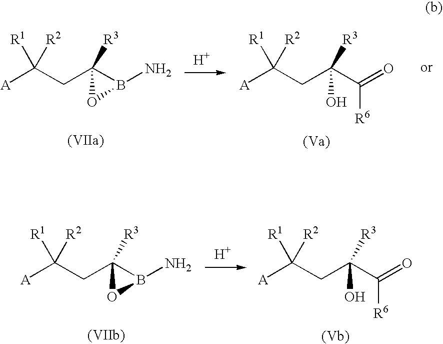

In still another aspect, a chiral carboxamide having Formula VIa or VIb can be produced Via a Darzens condensation (or also known as Darzens-Claisen reaction or glycidic ester condensation) (see;. e.g., T. Rosen, Comp. Org. Syn., Vol. 2, 409 (1991)) of an aldehyde or ketone with an chiral ester of an α-haloacid. One embodiment of such a condensation reaction is shown in Scheme 3. Typically, this reaction is carried out in a solvent (such as anhydrous THF) under an inert atmosphere, at low temperature (such as from −10° C. to room temperature) and in the presence of a basic condensation agent such as metal alcoholate, sodium amide, or metallic sodium. See; e.g., C. Kimura et al., Ind. Eng. Chem. Prod. Res. Dev., Vol. 22, 118 (1983).

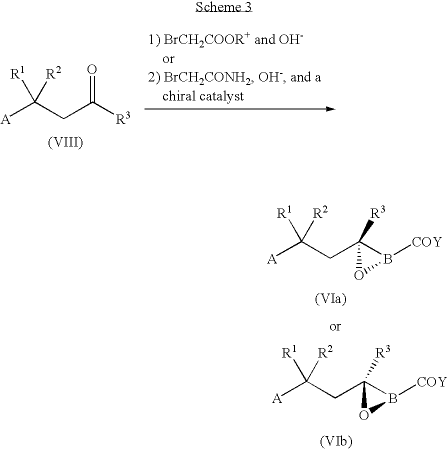

wherein Y is OR*, NH₂, or NHR*, and R* is a chiral auxiliary, and A, R¹, R², and R³ have the meanings disclosed herein above.

In one aspect of the present invention, a single substituted alcohol steroisomer having Formula Ia or Ib, substantially free of the other stereoisomer, can be produced according to a method comprising: (a) reacting a ketone having Formula III with a chiral ester of an α-haloacid under a basic condition to form a chiral epoxyester having Formula VIa or VIb

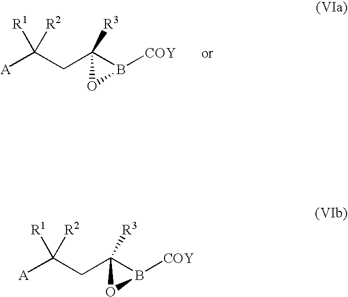

wherein Y is OR* and R* is a chiral auxiliary;
(b) reacting the chiral epoxyester having Formula VIa or VIb with ammonia or an amine to produce a chiral epoxycarboxamide having Formula IXa or IXb

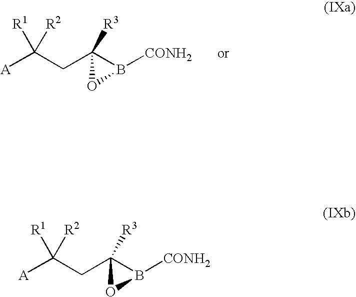

(c) converting the chiral epoxycarboxamide having Formula IXa or IXb to a chiral primary amine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

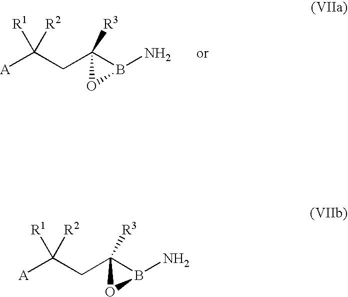

(d) reducing the chiral primary epoxyamine having Formula VIIa or VIIb to form a chiral hydroxyamine having Formula IVa or IVb

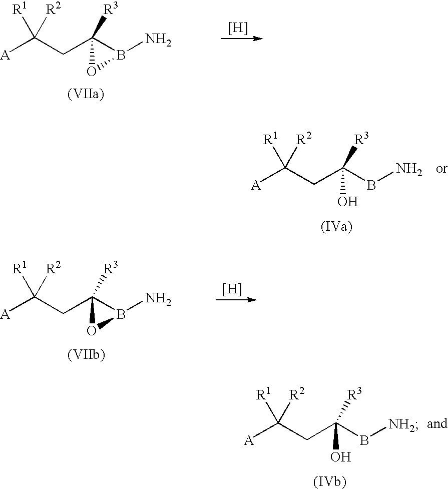

(e) reacting the chiral hydroxyamine having Formula IVa or IVb with a compound having a formula of Q-X under a base catalysis condition or under transition metal catalysis to produce the single substituted alcohol stereoisomer having Formula Ia or Ib;

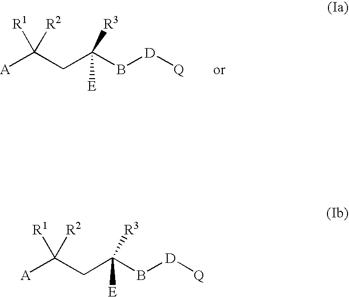

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ cycloalkyl group (or alternatively, C₃-C₆, or C₃-C₅, or C₃ cycloalkyl group); R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; B comprises a methylene or substituted methylene group, wherein a substituent on the methylene group is C₁-C₅ alkyl (or alternatively, C₁-C₃ alkyl), hydroxy, halogen, or amino; E is hydroxy; D is —NH— or —NR′—, wherein R′ comprises an unsubstituted or substituted C₁-C₁₅ (alternatively, C₁-C₁₀, or C₁-C₅, or C₁-C₃) linear or branched alkyl group; and X is a halogen (such as bromine, chlorine, fluorine, or iodine) or tosylate group.

In another aspect of the present invention, a single substituted alcohol steroisomer having Formula Ia or Ib, substantially free of the other stereoisomer, can be produced according to a method comprising: (a) reacting a ketone having Formula VIII with an amide of an α-haloacid under a basic condition in the presence of a chiral catalyst to form a chiral epoxycarboxamide having Formula IXa or IXb

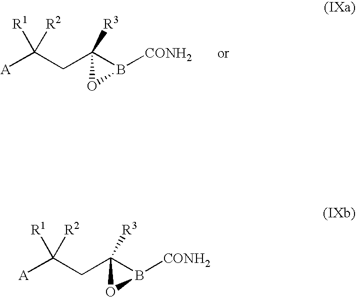

(b) converting the chiral epoxycarboxamide having Formula IXa or IXb to a chiral primary amine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

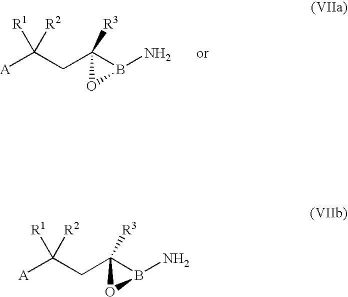

(c) reducing the chiral primary epoxyamine having Formula VIIa or VIIb to form a chiral hydroxyamine having Formula IVa or IVb

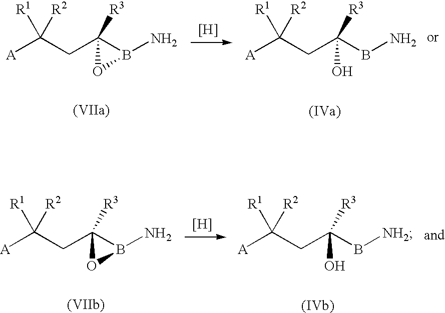

(d) reacting the chiral hydroxyamine having Formula IVa or IVb with a compound having a formula of Q-X under a base catalysis condition or under transition metal catalysis to produce the single substituted alcohol stereoisomer having Formula Ia or Ib;

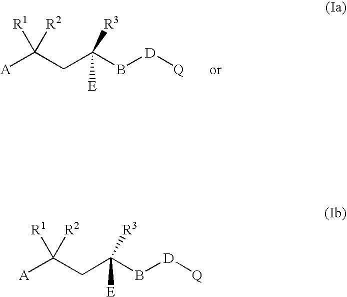

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ cycloalkyl group (or alternatively, C₃-C₆, or C₃-C₅, or C₃ cycloalkyl group); R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; B comprises a methylene or substituted methylene group, wherein a substituent on the methylene group is C₁-C₅ alkyl (or alternatively, C₁-C₃ alkyl), hydroxy, halogen, or amino; E is hydroxy; D is —NH— or —NR′—, wherein R′ comprises an unsubstituted or substituted C₁-C₁₅ (alternatively, C₁-C₁₀, or C₁-C₅, or C₁-C₃) linear or branched alkyl group; and X is a halogen (such as bromine, chlorine, fluorine, or iodine) or tosylate group.

In still another aspect of the present invention, a single substituted alcohol steroisomer having Formula Ia or Ib, substantially free of the other stereoisomer, can be produced according to a method comprising: (a) reacting a ketone having Formula VIII with a chiral ester of an α-haloacid under a basic condition to form a chiral epoxyester having Formula VIa or VIb

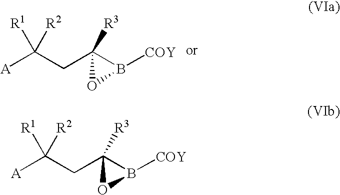

wherein Y is OR* and R* is a chiral auxiliary;
(b) reacting the chiral epoxyester having Formula VIa or VIb with ammonia or an amine to produce a chiral epoxycarboxamide having Formula IXa or IXb;

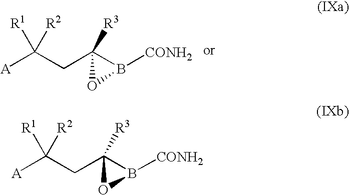

(c) converting the chiral epoxycarboxamide having Formula IXa or IXb to a chiral primary amine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite);

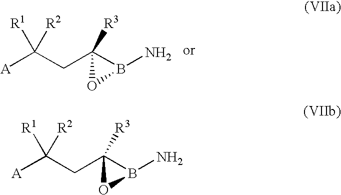

(d) converting the chiral primary amine having Formula VIIa or VIIb under an acidic condition, for example in an aqueous medium, to the chiral aldehyde or ketone having Formula Va or Vb; and

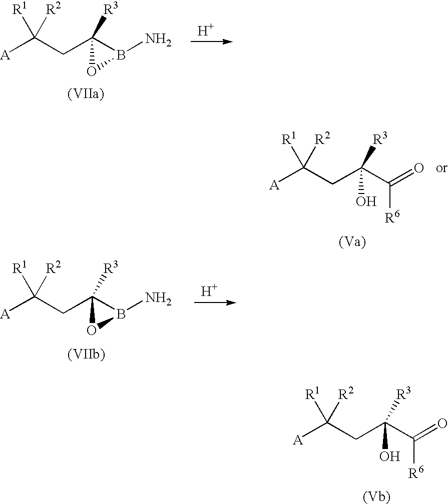

(e) reacting the chiral aldehyde or ketone having Formula Va or Vb with a compound having a formula of Q-NH₂ (or Q-NHR′) to produce the single substituted alcohol stereoisomer having Formula Ia or Ib;

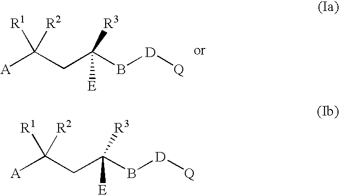

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₁₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ cycloalkyl group (or alternatively, C₃-C₆, or C₃-C₅, or C₃ cycloalkyl group); R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, L C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; R⁶ is hydrogen, C₁-C₅ alkyl (or alternatively, C₁-C₃ alkyl), hydroxy, halogen, or amino; B comprises a methylene or substituted methylene group, wherein a substituent on the methylene group is C₁-C₁₅ alkyl (or alternatively, C₁-C₃ alkyl), hydroxy, halogen, or amino; E is hydroxy; D is —NH— or —NR′—, wherein R′ comprises an unsubstituted or substituted C₁-C₁₅ (alternatively, C₁-C₁₀, or C₁-C₅, or C₁-C₃) linear or branched alkyl group; and X is a halogen (such as bromine, chlorine, fluorine, or iodine) or tosylate group.

In yet another aspect of the present invention, a single substituted alcohol steroisomer having Formula Ia or Ib, substantially free of the other stereoisomer, can be produced according to a method comprising: (a) reacting a ketone having Formula VIII with an amide of an α-haloacid under a basic condition in the presence of a chiral catalyst to form a chiral epoxycarboxamide having Formula IXa or IXb;

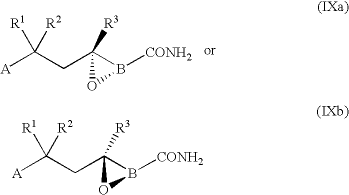

(b) converting the chiral epoxycarboxamide having Formula IXa or IXb to a chiral primary epoxyamine having Formula VIIa or VIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite);

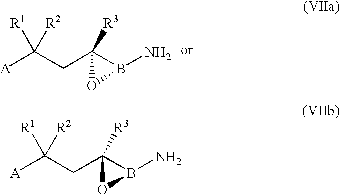

(c) converting the chiral primary epoxyamine having Formula VIIa or VIIb under an acidic condition, for example in an aqueous acidic medium, to the chiral aldehyde or ketone having Formula Va or Vb; and

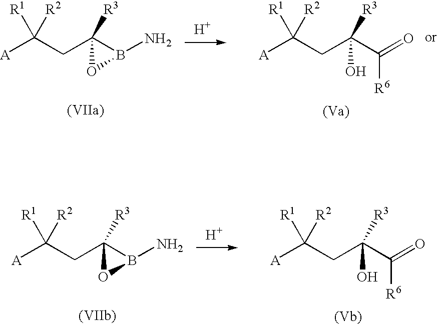

(e) reacting the chiral aldehyde or ketone having Formula Va or Vb with a compound having a formula of Q-NH₂ (or Q-NHR′) to produce the single substituted alcohol stereoisomer having Formula Ia or Ib;

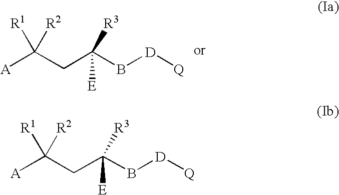

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; or R¹ and R² together form an unsubstituted or substituted C₃-C₁₅ cycloalkyl group (or alternatively, C₃-C₆, or C₃-C₅, or C₃ cycloalkyl group); R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclic groups; R⁶ is hydrogen, C₁-C₅ alkyl (or alternatively, C₁-C₃ alkyl), hydroxy, halogen, or amino; B comprises a methylene or substituted methylene group, wherein a substituent on the methylene group is C₁-C₅ alkyl (or alternatively, C₁-C₃ alkyl), hydroxy, halogen, or amino; E is hydroxy; D is —NH— or —NR′—, wherein R′ comprises an unsubstituted or substituted C₁-C₁₅ (alternatively, C₁-C₁₀, or C₁-C₅, or C₁-C₃) linear or branched alkyl group; and X is a halogen (such as bromine, chlorine, fluorine, or iodine) or tosylate group.

In still another aspect, the present invention provides a method for producing stereoisomeric DIGRA compounds having Formula IIa, IIb, IIc, or IId,

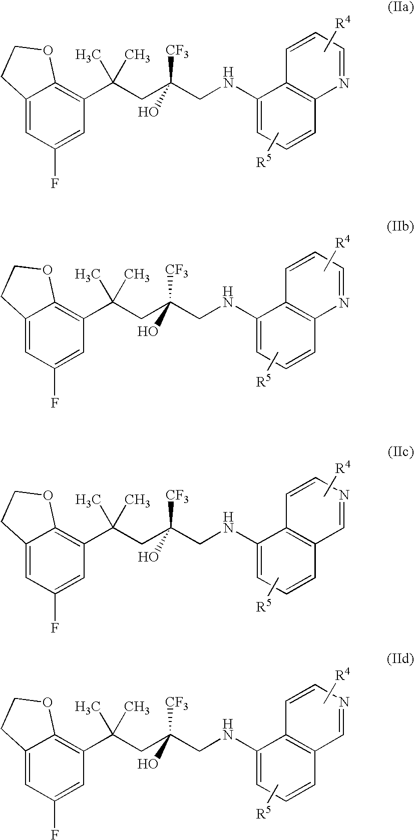

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In one embodiment of the present invention, a single substituted alcohol steroisomer having Formula IIa or IIb, substantially free of the other stereoisomer, can be produced according to a method comprising: (a) reacting a ketone having Formula X with a chiral ester of an α-haloacid (such as BrCH₂COOR*, wherein R* is a chiral auxiliary) under a basic condition to form a chiral epoxyester having Formula XIa or XIb

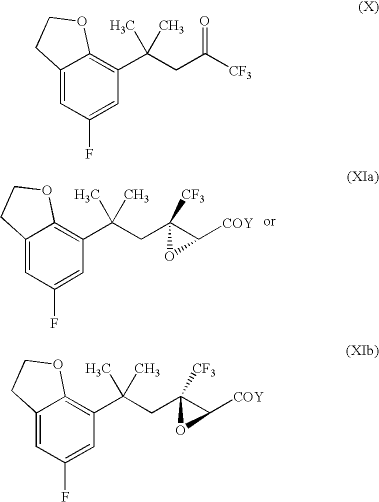

wherein Y is OR* and R* is a chiral auxiliary;
(b) reacting the chiral epoxyester having Formula XIa or XIb with ammonia or an amine to produce a chiral epoxycarboxamide having Formula XIIa or XIIb;

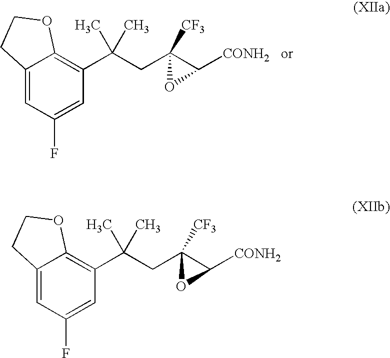

(c) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

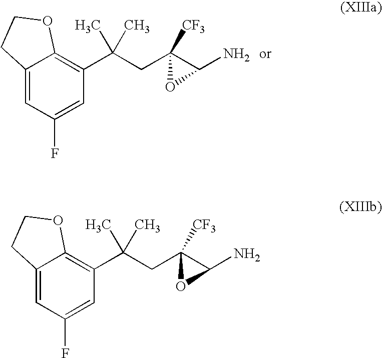

(d) reducing the chiral primary epoxyamine having Formula XIIIa or XIIIb to form a chiral hydroxyamine having Formula XIVa or XIVb

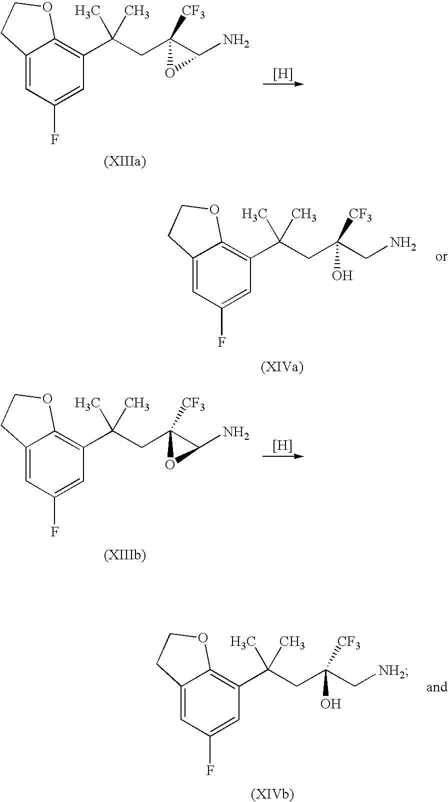

(e) reacting the chiral hydroxyamine having Formula XIVa or XIVb with a compound having a formula of Q-X under a base catalysis condition or under transition metal catalysis to produce the single substituted alcohol stereoisomer having Formula IIa or IIb;

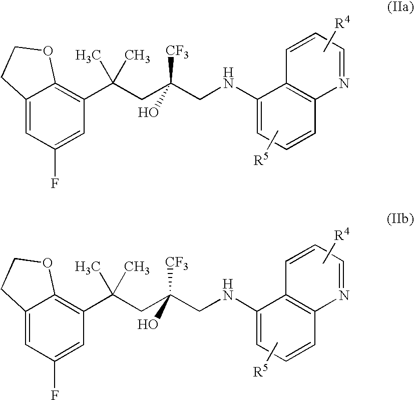

wherein Q is the quinolin-5-yl group which is unsubstituted or substituted at one or more positions 2, 3, 4, 6, 7, or 8; and X is a halogen (such as bromine, chlorine, fluorine, or iodine) or tosylate group attached to the quinolinyl group at the 5 position.

In another embodiment of the present invention, a single substituted alcohol steroisomer having Formula IIa or IIb, substantially free of the other stereoisomer, can be produced according to a method comprising: (a) reacting a ketone having Formula X with a chiral ester of an α-haloacid (such as BrCH₂COOR*, wherein R* is a chiral auxiliary) under a basic condition to form a chiral epoxyester having Formula XIa or XIb

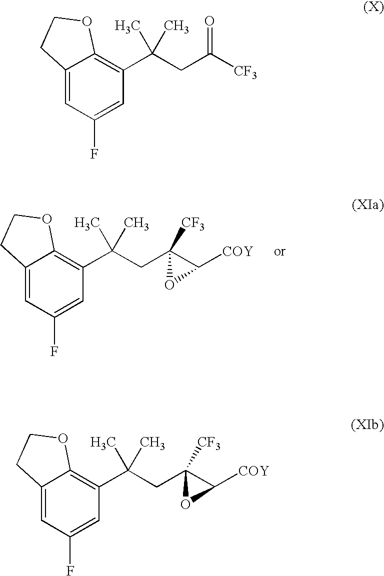

wherein Y is OR* and R* is a chiral auxiliary;
(b) reacting the chiral epoxyester having Formula XIa or XIb with ammonia or an amine to produce a chiral epoxycarboxamide having Formula XIIa or XIIb;

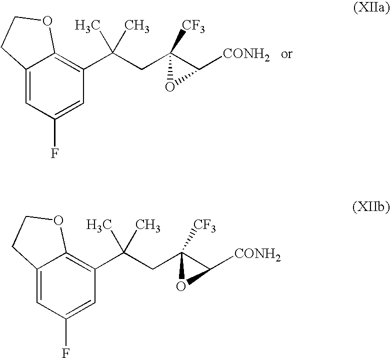

(c) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

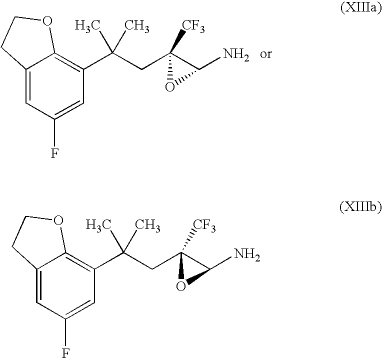

(d) converting the chiral primary epoxyamine having Formula XIIIa or XIIIb under an acidic condition to form a chiral chiral aldehyde having Formula XVa or XVb

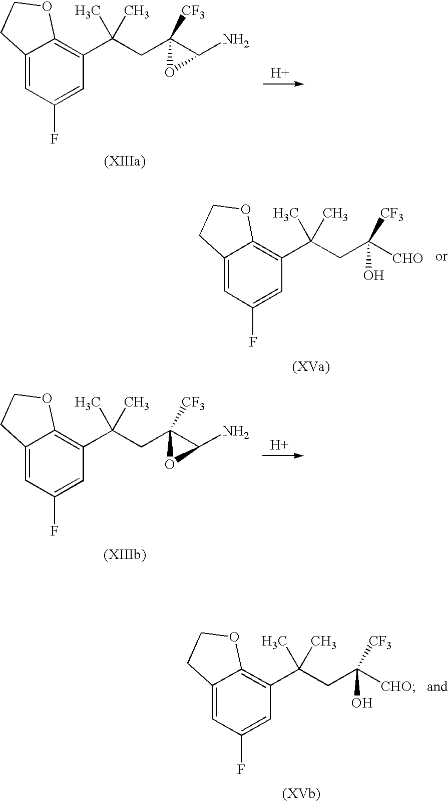

(e) reacting the chiral aldehyde having Formula XVa or XVb with a compound having a formula of Q-NH₂ or Q-NR′ under a base catalysis condition or under transition metal catalysis to produce the single substituted alcohol stereoisomer having Formula IIa or IIb;

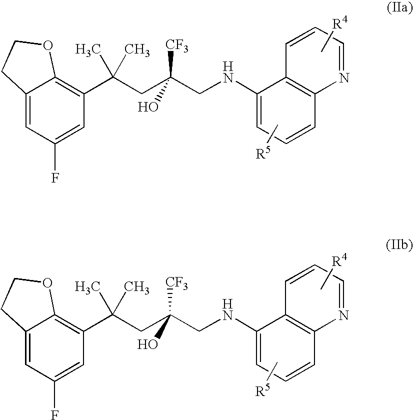

wherein Q is the quinolin-5-yl group which is unsubstituted or substituted at one or more positions 2, 3, 4, 6, 7, or 8; and R′ is an unsubstituted or substituted C₁-C₅ linear or branched alkyl group.

In another embodiment, compounds having Formula IIc or IId can be prepared according to either of the two methods described immediately above by replacing the quinolinyl group with the isoquinolinyl group.

In still another embodiment of the present invention, a single substituted alcohol steroisomer having Formula IIa or IIb, substantially free of the other stereoisomer, can be produced according to a method comprising: (a) reacting a ketone having Formula X with a with an amide of an α-haloacid (such as BrCH₂CONH₂ or ClCH₂CONH₂) under a basic condition in the presence of a chiral catalyst to form a chiral epoxycarboxamide having Formula having Formula XIa or XIb

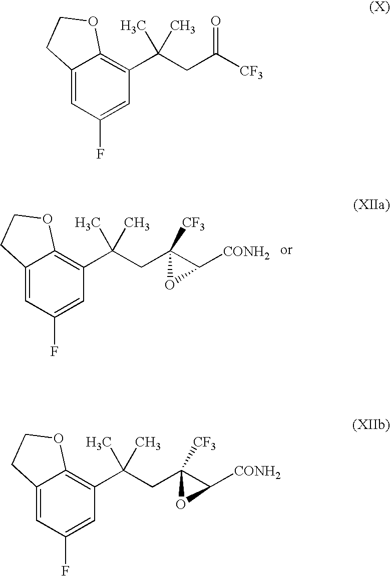

(b) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

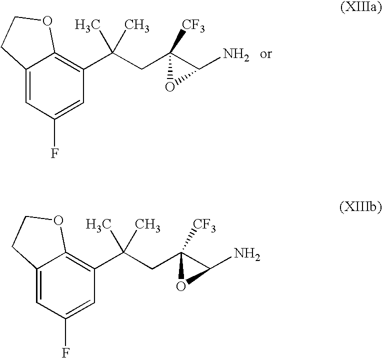

(c) reducing the chiral primary epoxyamine having Formula XIIIa or XIIIb to form a chiral hydroxyamine having Formula XIVa or XIVb

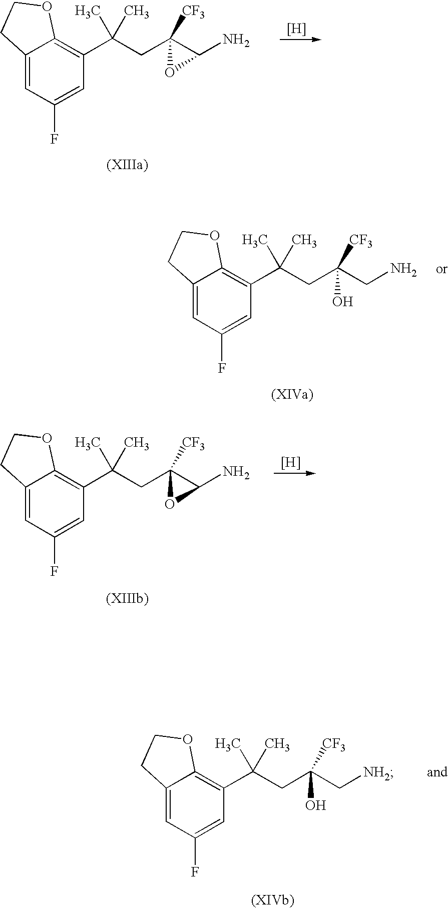

(d) reacting the chiral hydroxyamine having Formula XIVa or XIVb with a compound having a formula of Q-X under a base catalysis condition or under transition metal catalysis to produce the single substituted alcohol stereoisomer having Formula IIa or IIb;

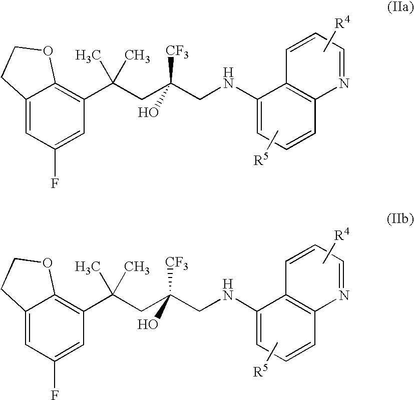

wherein Q is the quinolin-5-yl group which is unsubstituted or substituted at one or more positions 2, 3, 4, 6, 7, or 8; and X is a halogen (such as bromine, chlorine, fluorine, or iodine) or tosylate group attached to the quinolinyl group at the 5 position.

In still another embodiment of the present invention, a single substituted alcohol steroisomer having Formula IIa or IIb, substantially free of the other stereoisomer, can be produced according to a method comprising: (a) reacting a ketone having Formula X with a with an amide of an α-haloacid (such as BrCH₂CONH₂ or ClCH₂CONH₂) under a basic condition in the presence of a chiral catalyst to form a chiral epoxycarboxamide having Formula having Formula XIa or XIb

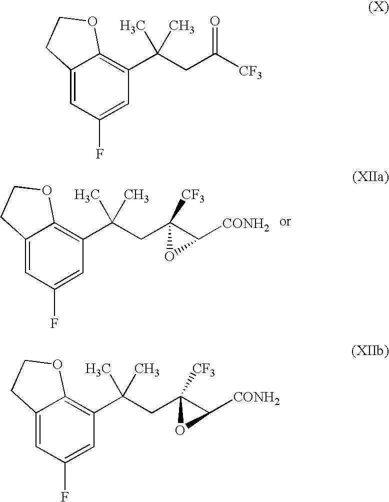

(b) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

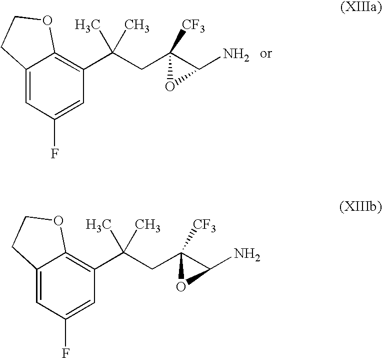

(c) converting the chiral primary epoxyamine having Formula XIIIa or XIIIb under an acidic condition to a chiral aldehyde having Formula XVa or XVb

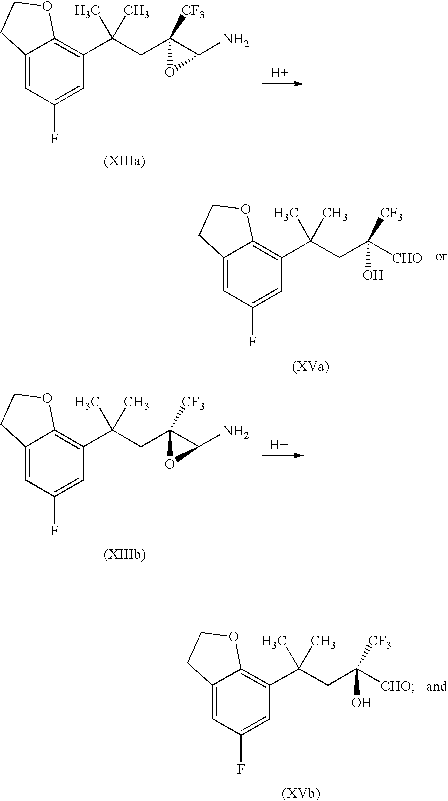

(d) reacting the chiral aldehyde having Formula XVa or XVb with a compound having a formula of Q-NH₂ or Q-NR′ to produce the single substituted alcohol stereoisomer having Formula IIa or IIb;

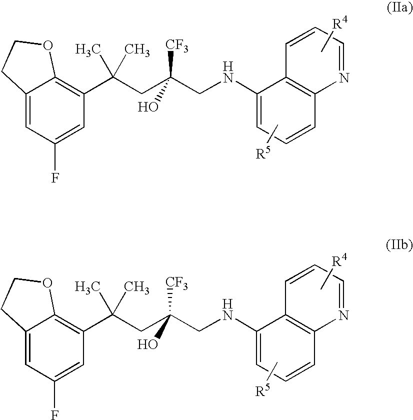

wherein Q is the quinolin-5-yl group which is unsubstituted or substituted at one or more positions 2, 3, 4, 6, 7, or 8; and R′ is an unsubstituted or substituted C₁-C₅ linear or branched alkyl group.

In still another embodiment, compounds having Formula IIc or IId can be prepared according to either of the two methods described immediately above by replacing the quinolinyl group with the isoquinolinyl group.

In still another aspect, the present invention provides a method for producing stereoisomeric DIGRA compounds having Formula IIIa, IIIb, IIIc, or IIId.

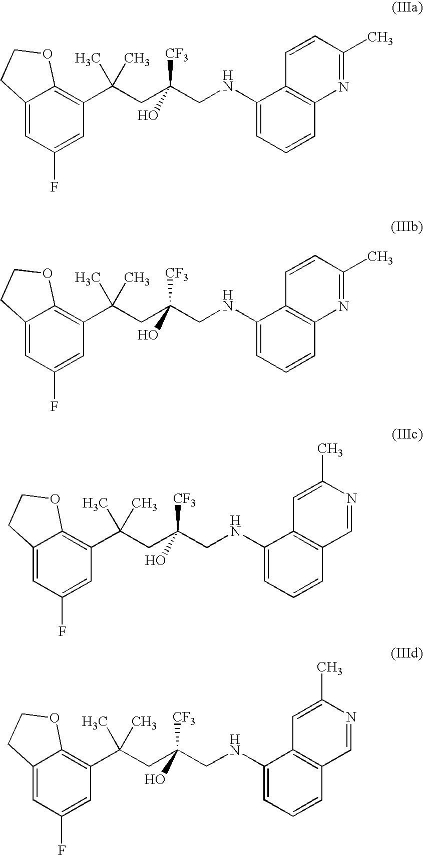

In yet another embodiment of the present invention, a single substituted alcohol steroisomer having Formula IIIa or IIIb, substantially free of the other stereoisomer, can be produced according to a method comprising: (a) reacting a ketone having Formula X with a chiral ester of an α-haloacid (such as BrCH₂COOR*, wherein R* is a chiral auxiliary) under a basic condition to form a chiral epoxyester having Formula XIa or XIb

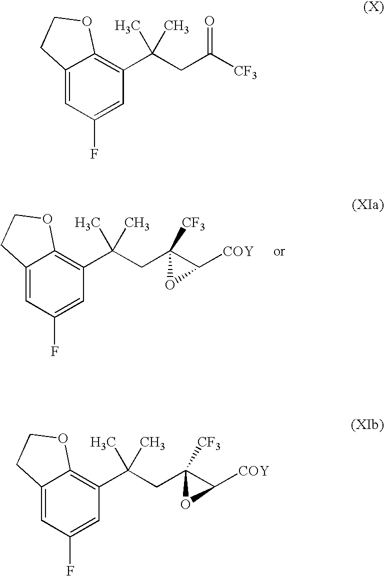

wherein Y is OR* and R* is a chiral auxiliary;
(b) reacting the chiral epoxyester having Formula XIa or XIb with ammonia or an amine to produce a chiral epoxycarboxamide having Formula XIIa or XIIb

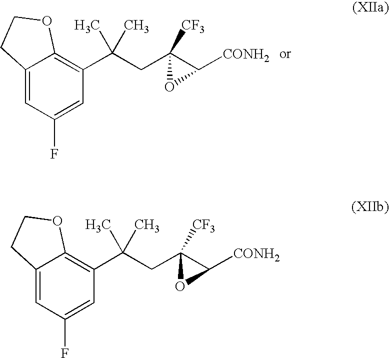

(c) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

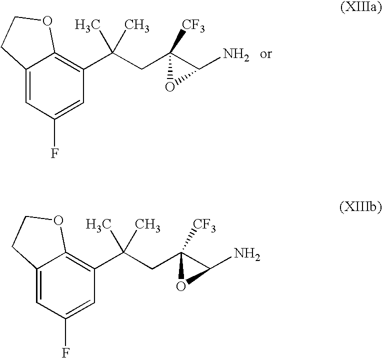

(d) reducing the chiral primary epoxyamine having Formula XIIIa or XIIIb to form a chiral hydroxyamine having Formula XIVa or XIVb

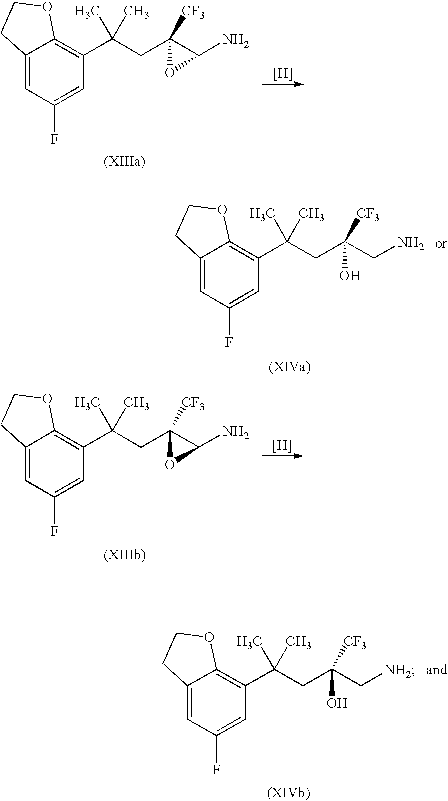

(e) reacting the chiral hydroxyamine having Formula XIVa or XIVb with 2-methyl-5-halo-quinoline under a base catalysis condition or under transition metal catalysis to produce the single substituted alcohol stereoisomer having Formula IIIa or IIIb;

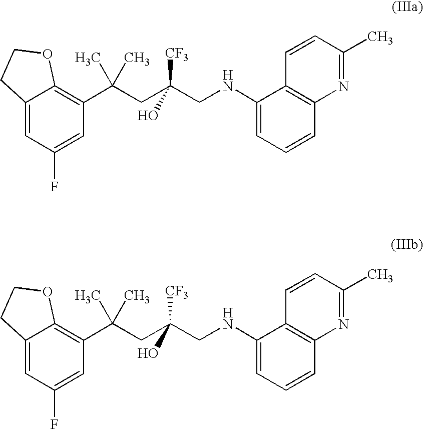

wherein the halo substituent at the 5 position on the substituted quinoline is selected from the group consisting bromine, chlorine, fluorine, and iodine.

In yet another embodiment of the present invention, a single substituted alcohol steroisomer having Formula IIIa or IIIb, substantially free of the other stereoisomer, can be produced according to a method comprising: (a) reacting a ketone having Formula X with a chiral ester of an α-haloacid (such as BrCH₂COOR*, wherein R* is a chiral auxiliary) under a basic condition to form a chiral epoxyester having Formula XIa or XIb

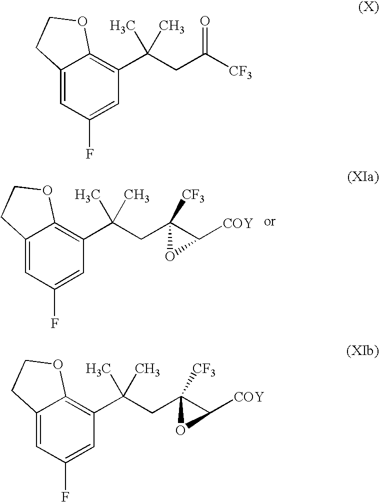

wherein Y is OR* and R* is a chiral auxiliary;
(b) reacting the chiral epoxyester having Formula XIa or XIb with ammonia or an amine to produce a chiral epoxycarboxamide having Formula XIIa or XIIb

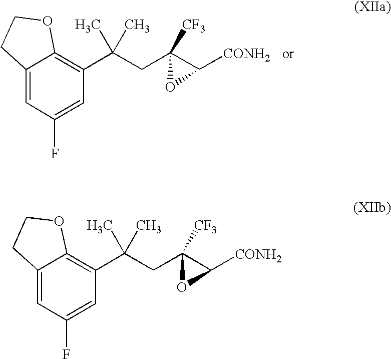

(c) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

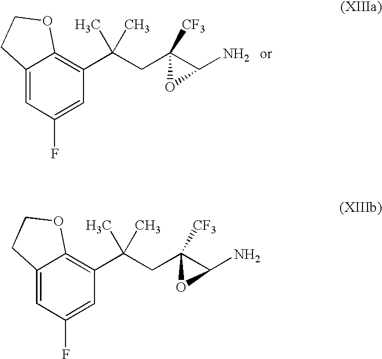

(d) converting the chiral primary epoxyamine having Formula XIIIa or XIIIb to form a chiral aldehyde having Formula XVa or XVb

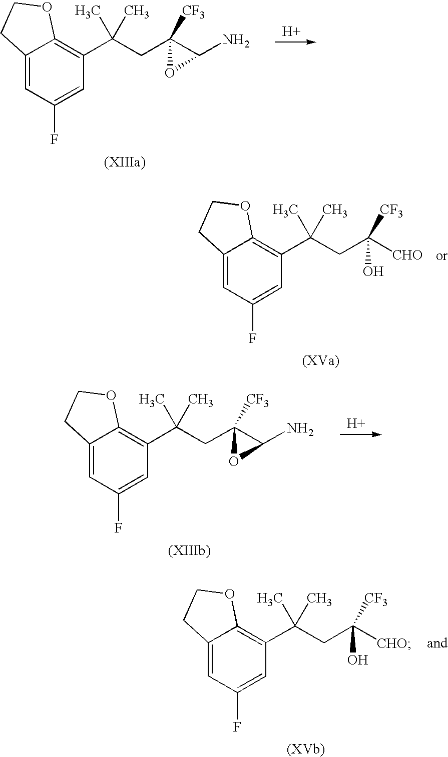

(e) reacting the chiral aldehyde having Formula XVa or XVb with 2-methyl-5-amino-quinoline to produce the single substituted alcohol stereoisomer having Formula IIIa or IIIb.

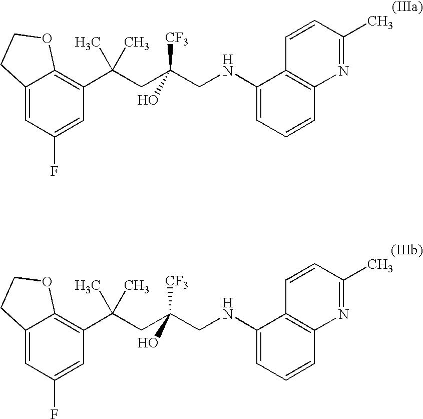

In another embodiment, compounds having Formula IIIc or IIId can be prepared according to either of the two methods described immediately above by replacing the quinolinyl group with the isoquinolinyl group.

In a further embodiment of the present invention, a single substituted alcohol steroisomer having Formula IIIa or IIIb, substantially free of the other stereoisomer, can be produced according to a method comprising: (a) reacting a ketone having Formula X with a with an amide of an α-haloacid (such as BrCH₂CONH₂ or ClCH₂CONH₂) under a basic condition in the presence of a chiral catalyst to form a chiral epoxycarboxamide having Formula having Formula XIa or XIb

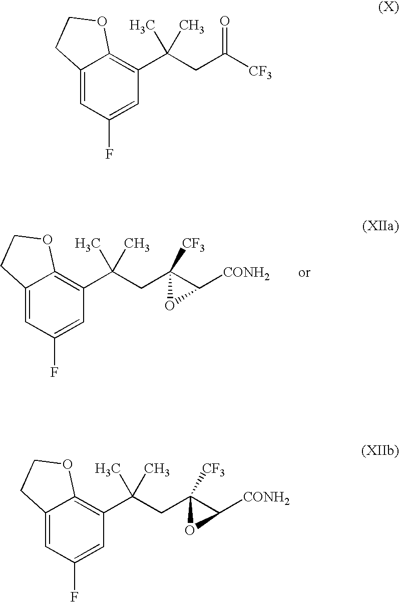

(b) converting the chiral epoxycarboxamide having Formula XIIa or XIIb to a chiral primary epoxyamine having Formula XIIIa or XIIIb through a Hoffman rearrangement under a halogen (such as bromine, chlorine, fluorine, or iodine; preferably, bromine) and in the presence of a base (such as NaOH or KOH), or upon treatment with an alkali hypohalite (such as alkali hypobromite, hypochlorite, hypofluorite, or hypoiodite)

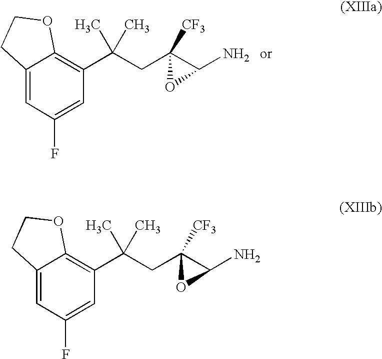

(c) converting the chiral primary epoxyamine having Formula XIIIa or XIIIb to form a chiral aldehyde having Formula XVa or XVb

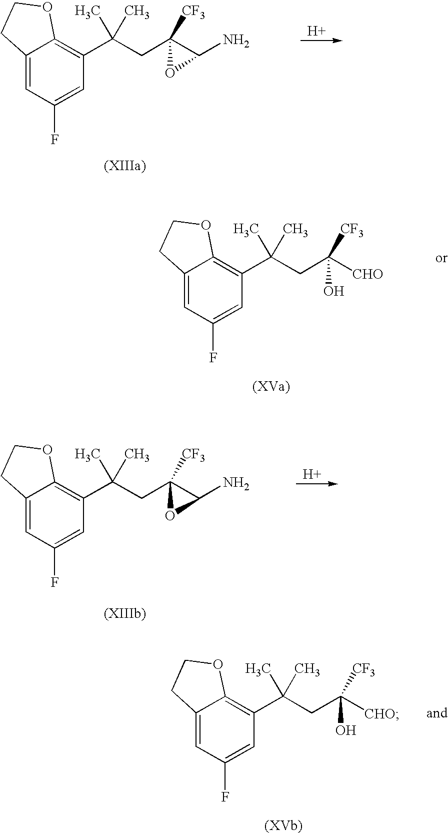

(d) reacting the chiral aldehyde having Formula XVa or XVb with 2-methyl-5-amino-quinoline to produce the single substituted alcohol stereoisomer having Formula IIIa or IIIb.

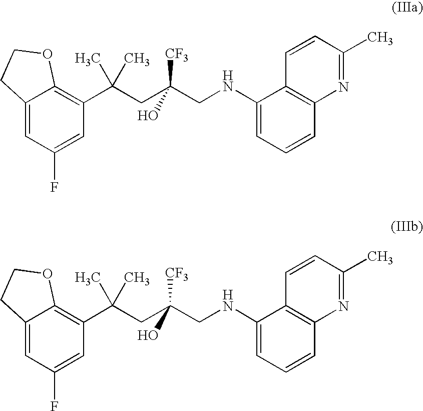

In still another embodiment, compounds having Formula IIIc or IIId can be prepared according to the method described immediately above by replacing the quinolinyl group with the isoquinolinyl.

In still another aspect, the present invention provides a stereoisomeric compound having Formula Ia, Ib, IIa, IIb, IIIa, or IIIb and a method for their production, whence a prodrug, a pharmaceutically acceptable salt, or a pharmaceutically acceptable ester of such a stereoisomeric compound may be prepared.

In yet another aspect, a method of the present invention yields such a steroisomeric compound in substantially pure form (i.e., subbstantially free of the other steroisomer of the racemic mixture).

Non-limiting examples of compounds having Formula Ia or Ib that may be produced by a method of the present invention include 5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-1-methylisoquinoline, 5-[4-(5-fluoro-2,3-dihydrobenzofuran-7-yl)-2-hydroxy-4-methyl-2-trifluoromethyl-pentylamino]isoquinol-1(2H)-one, 5-[4-(5-fluoro-2,3-dihydrobenzofuran-7-yl)-2-hydroxy-4-methyl-2-trifluoromethyl-pentylamino]-2,6-dimethylquinoline, 5-[4-(5-fluoro-2,3-dihydrobenzofuran-7-yl)-2-hydroxy-4-methyl-2-trifluoromethyl-pentylamino]-6-chloro-2-methylquinoline, 5-[4-(5-fluoro-2,3-dihydrobenzofuran-7-yl)-2-hydroxy-4-methyl-2-trifluoromethyl-pentylamino]isoquinoline, 5-[4-(5-fluoro-2,3-dihydrobenzofuran-7-yl)-2-hydroxy-4-methyl-2-trifluoromethyl-pentylamino]quinoline, 5-[4-(2,3-dihydro-5-fluoro-7-benzofuranyl)-2-hydroxy-4-methyl-2-trifluoromethyl-pentylamino]quinolin-2[1H]-one, 6-fluro-5-[4-(5-fluoro-2,3-dihydrobenzofuran-7-yl)-2-hydroxy-4-methyl-2-trifluoromethyl-pentylamino]-2-methylquinoline, 8-fluoro-,5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-2-methylisoquinol-1-[2H]-one, and enantiomers thereof.

In yet another embodiment, the present invention provide a method for producing a stereoisomeric DIGRA compound having Formula Ia or Ib, wherein

(a) A is an aryl or heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is the trifluoromethyl group;

(d) B is a methylene or substituted methylene group, wherein a substituent group of B is independently C₁-C₃ alkyl, hydroxy, halogen, amino, or oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q is an azaindolyl group optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, halogen, hydroxy, oxo, cyano, amino, and trifluoromethyl.

Non-limiting examples of these compounds include 1,1,1-trifluoro-4-(5-fluoro-2-methoxyphenyl)-4-methyl-2-(((1H-pyrrolo[2,3-c]pyridin-2-yl)methylamino)methyl)pentan-2-ol; 1,1,1-trifluoro-4-(5-fluoro-2-methoxyphenyl)-4-methyl-2-(((1H-pyrrolo[3,2-c]pyridin-2-yl)methylamino)methyl)pentan-2-ol; 1,1,1-trifluoro-4-methyl-4-phenyl-2-(((1H-pyrrolo[2,3-c]pyridin-2-yl)methylamino)methyl)pentan-2-ol; 1,1,1-trifluoro-4-(4-fluoro-2-methoxyphenyl)-4-methyl-2-((1H-pyrrolo[2,3-c]pyridin-2-yl)methylamino)methyl)pentan-2-ol; 1,1,1-trifluoro-4-methyl-4-phenyl-2-(((1H-pyrrolo[3,2-c]pyridin-2-yl)methylamino)methyl)pentan-2-ol; 1,1,1-trifluoro-4-(4-fluoro-2-methoxyphenyl)-4-methyl-2-(((1H-pyrrolo[3,2-c]pyridin-2-yl)methylamino)methyl)pentan-2-ol; and 1,1,1-trifluoro-4-(5-fluoro-2-methoxyphenyl)-4-methyl-2-(((3-methyl-1H-pyrrolo[2,3-c]pyridin-2-yl)methylamino)methyl)pentan-2-ol.

In still another embodiment, the present invention provide a method for producing a stereoisomeric DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) B is a methylene or substituted methylene group, wherein one or two substituents on the methylene group is C₁-C₅ alkyl (or alternatively, C₁-C₃ alkyl), hydroxy, amino, or oxo group;

(d) R³ is a carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a methylated benzoxazinone.

Non-limiting examples of these compounds include 6-[2-benzyl-4-(5-fluoro-2-methoxyphenyl)-2-hydroxy-4-methylpentylamino]-(4-methyl-1-oxo-1H-benzo[d][1,2]oxazine); 7-[2-benzyl-4-(5-fluoro-2-hydroxyphenyl)-2-hydroxy-4-methylpentylamino]-(4-methyl-1-oxo-1H-benzo[d][1,2]oxazine); 6-[2-cyclohexylmethyl-4-(5-fluoro-2-methoxyphenyl)-2-hydroxy-4-methylpentylamino]-(4-methyl-1-oxo-1H-benzo[d][1,2]oxazine); 6-[2-cyclohexylmethyl-4-(5-fluoro-2-hydroxyphenyl)-2-hydroxy-4-methylpentylamino]-(4-methyl-1-oxo-1H-benzo[d][1,2]oxazine); 5-benzyl-5-methyl-3-hydroxy-3-trifuoromethyl-hexanoic acid-(4-methyl-1-oxo-1H-benzo[d][1,2]oxazin-6-yl)amide; and 5-(2-methoxyphenyl)-3-cyclohexylmethyl-3-hydroxy-5-methylhexanoic acid-(4-methyl-1-oxo-1H-benzo[d][1,2]oxazin-6-yl)amide.

In still another embodiment, the present invention provide a method for producing a stereoisomeric DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is C₁-C₁₀ alkyl or substituted C₁-C₁₀ alkyl group (in certain embodiments, R³ is a partially or completely halogenated C₁-C₁₀ alkyl group, and in certain other embodiments, R³ is the trifluoromethyl group);

(d) B is a methylene or substituted methylene group, wherein a substituent group of B is independently C₁-C₃ alkyl, hydroxy, halogen, amino, or oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, and trifluoromethyl.

Non-limiting examples of these compounds include 2-(3,5-difluorobenzylamino)-1,1,1-trifluoro-4-(5-fluoro-2-methoxyphenyl)-4-methylpentan-2-ol; 2-biphenyl-4-ylmethyl-2-hydroxy-1,1,1-trifluoro-4-(5-fluoro-2-methoxyphenyl)-4-methylpentane; 2-(3,5-dimethylbenzylamino)-1,1,1-trifluoro-4-(5-fluoro-2-methoxyphenyl)-4-methylpentan-2-ol; 2-(3-bromobenzylamino)-1,1,1-trifluoro-4-(5-fluoro-2-methoxyphenyl)-4-methylpentan-2-ol; 2-(3,5-dichlorobenzylamino)-1,1,1-trifluoro-4-(5-fluoro-2-methoxyphenyl)-4-methylpentan-2-ol; 2-(3,5-bis-trifluoromethylbenzylamino)-1,1,1-trifluoro-4-(5-fluoro-2-methoxyphenyl)-4-methylpentan-2-ol; 1,1,1-trifluoro-4-(5-fluoro-2-methoxyphenyl)-2-(3-fluoro-5-trifluoromethylbenzylamino)-4-methylpentan-2-ol; 2-(3-chloro-2-fluoro-5-trifluoromethylbenzylamino)-1,1,1-trifluoro-4-(5-fluoro-2-methoxyphenyl)-4-methylpentan-2-ol; 2-(3,5-dibromobenzylamino)-1,1,1-trifluoro-4-(5-fluoro-2-methoxyphenyl)-4-methylpentan-2-ol; 1,1,1-trifluoro-4-(5-fluoro-2-methoxyphenyl)-2-(2-fluoro-3-trifluoromethylbenzylamino)-4-methylpentan-2-ol; 1,1,1-trifluoro-4-(5-fluoro-2-methoxyphenyl)-2-(2-fluoro-5-trifluoromethylbenzylamino)-4-methylpentan-2-ol.

In still another embodiment, the present invention provide a method for producing a stereoisomeric DIGRA compound having Formula Ia or Ib, wherein

(a) A is an aryl, heteroaryl, or C₅-C₁₅ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen, C₁-C₅ alkyl, C₅-C₁₅ arylalkyl, or R¹ and R² together with the carbon atom they are commonly attached to form a C₃-C₈ spiro cycloalkyl ring;

(c) R³ is the trifluoromethyl group;

(d) B is methylene or substituted methylene group, wherein one or two substituents on the methylene group are independently C₁-C₅ alkyl (or alternatively, C₁-C₃ alkyl), hydroxy, amino, halogen, or oxo group;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is a hydroxy group; and

(g) Q comprises a quinoline, isoquinoline, pyrrolidine, morpholine, thiomorpholine, piperazine, piperidine, 1H-pyridin-4-one, 1H-pyridin-2-one, 1H-pyridin-4-ylideneamine, 1H-quinolin-4-ylideneamine, pyran, tetrahydropyran, 1,4-diazepane, 2,5-diazabicyclo[2.2.1]heptane, 2,3,4,5-tetrahydrobenzo[b][1,4]diazepine, dihydroquinoline, tetrahydroquinoline, 5,6,7,8-tetrahydro-1H-quinolin-4-one, tetrahydroisoquinoline, decahydroisoquinoline, 2,3-dihydro-1H-isoindole, 2,3-dihydro-1H-indole, chroman, 1,2,3,4-tetrahydroquinoxaline, 1,2-dihydroindazol-3-one, 3,4-dihydro-2H-benzo[1,4]oxazine, 4H-benzo[1,4]thiazine, 3,4-dihydro-2H-benzo[1,4]thiazine, 1,2-dihydrobenzo[d][1,3]oxazin-4-one, 3,4-dihydrobenzo[1,4]oxazin-4-one, 3H-quinazolin-4-one, 3,4-dihydro-1H-quinoxalin-2-one, 1H-quinnolin-4-one, 1H-quinazolin-4-one, 1H-[1,5]naphthyridin-4-one, 5,6,7,8-tetrahydro-1H-[1,-5]naphthyridin-4-one, 2,3-dihydro-1H-[1,5]naphthyridin-4-one, 1,2-dihydropyrido[3,2-d][1,3]oxazin-4-one, pyrrolo[3,4-c]pyridine-1,3-dione, 1,2-dihydropyrrolo[3,4-c]pyridin-3-one, or tetrahydro[b][1,4]diazepinone group, each optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, oxo, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from C₁-C₃ alkyl, C₁-C₃ alkoxy, C₁-C₃ alkoxycarbonyl, acyl, aryl, benzyl, heteroaryl, heterocyclyl, halogen, hydroxy, oxo, cyano, amino wherein the nitrogen atom is optionally independently mono- or di-substituted by C₁-C₅ alkyl, or ureido wherein either nitrogen atom is optionally independently substituted with C₁-C₅ alkyl.

Non-limiting examples of these compounds include 2-((2,6-dimethylmorpholin-4-yl)methylamino)methyl)-1,1,1-trifluoro-4-(5-fluoro-2-methoxyphenyl)-4-methylpentan-2-ol; 6-[(4-(5-fluoro-2-methoxyphenyl)-2-hydroxy-4-methyl-2-trifluoromethylpentyl)amino]-(1H-quinolin-4-one); 3-[(4-(5-fluoro-2-methoxyphenyl)-2-hydroxy-4-methyl-2-trifluoromethylpentyl)amino]-(5-methylpiperidin-4-one); 6-[(4-(5-fluoro-2-methoxyphenyl)-2-hydroxy-4-methyl-2-trifluoromethylpentyl)amino]-(3-methyl-1H-quinolin-4-one); 6-[(4-(5-fluoro-2-methoxyphenyl)-2-hydroxy-4-methyl-2-trifluoromethylpentyl)amino]-(2,3-dihydro-1H-quinolin-4-one); 6-[(4-(4-fluorophenyl)-2-hydroxy-4-methyl-2-trifluoromethylpentyl)amino]-(1H-quinolin-4-one); 5-[(4-(3-fluorophenyl)-2-hydroxy-4-methyl-2-trifluoromethylpentyl)amino]-(1H-quinolin-3-one); 6-[(4-(4-fluoro-2-hydroxyphenyl)-2-hydroxy-4-methyl-2-trifluoromethylpentyl)amino]-(1H-quinolin-4-one); 5-[(4-phenyl-2-hydroxy-4-methyl-2-trifluoromethylpentyl)amino]-(1H-quinolin-3-one); 7-[(4-(5-fluoro-2,3-dihydrobenzofuran-7-yl)-2-hydroxy-4-methyl-2-trifluoromethylpentyl)amino]-(1H-quinolin-4-one); 6-[(4-(5-bromo-2,3-dihydrobenzofuran-7-yl)-2-hydroxy-4-methyl-2-trifluoromethylpentyl)amino]-(1H-quinolin-4-one); 6-[(4-(5-methyl-2,3-dihydrobenzofuran-7-yl)-2-hydroxy-4-methyl-2-trifluoromethylpentyl)amino]-(1H-quinolin-4-one); 6-[(4-(5-chloro-2,3-dihydrobenzofuran-7-yl)-2-hydroxy-4-methyl-2-trifluoromethylpentyl)amino]-(1H-quinolin-4-one); 6-[(4-(2,3-dihydrobenzofuran-7-yl)-2-hydroxy-4-methyl-2-trifluoromethylpentyl)amino]-(1H-quinolin-4-one); 6-[(4-(5-fluoro-2-hydroxyphenyl)-2-hydroxy-4-methyl-2-trifluoromethylpentyl)amino]-(1H-[1,5]naphthyridin-4-one); 1-[(4-(5-fluoro-2-methoxyphenyl)-2-hydroxy-2,4-dimethylpentyl)amino]-(3,5-dimethyl-1H-pyridin-4-one); 6-[(2-hydroxy-4-(2-methoxy-5-thiophen-2-ylphenyl)-4-methyl-2-trifluoromethylpentyl)amino]-(1H-quinolin-4-one); 6-[(4-(6-bromobenzo[1,3]dioxol-4-yl)-2-hydroxy-4-methyl-2-trifluoromethylpentyl)amino]-(1H-quinolin-4-one); 7-[(4-(5-fluoro-2-hydroxyphenyl)-2-hydroxy-4-methyl-2-trifluoromethylpentyl)amino]-(3-methyl-1H-quinolin-4-one); 6-[(2-hydroxy-4-(4-hydroxybiphenyl-3-yl)-4-methyl-2-trifluoromethylpentyl)amino]-(1H-quinolin-4-one); 6-{(4-[5-(3,5-dimethylisoxazol-4-yl)-2-hydroxyphenyl]-2-hydroxy-4-methyl-2-trifluoromethylpentyl)amino}-(1H-quinolin-4-one); 2-[(2-hydroxy-4-(2-hydroxy-5-thiophen-3-ylphenyl)-4-methyl-2-trifluoromethylpentyl)amino]-(1H-quinolin-4-one); 6-{(4-[5-(3,5-dimethylisoxazol-4-yl)-2-methoxyphenyl]-2-hydroxy-4-methyl-2-trifluoromethylpentyl)amino}-(1H-quinolin-4-one); 2-[(2-hydroxy-4-methyl-4-(3-pyridin-3-ylphenyl)-2-trifluoromethylpentyl)amino]-(1H-quinolin-4-one); 6-[(2-hydroxy-4-(2-methoxy-5-thiophen-3-ylphenyl)-4-methyl-2-trifluoromethylpentyl)amino]-(1H-quinolin-4-one); 7-[(4-(5-furan-3-yl-2-methoxyphenyl)-2-hydroxy-4-methyl-2-trifluoromethylpentyl)amino]-(1H-quinolin-4-one); 6-[(2-hydroxy-4-(4-methoxybiphenyl-3-yl)-4-methyl-2-trifluoromethylpentyl)amino]-(1H-quinolin-4-one); 6-[(4-(5-acetyl-2-hydroxyphenyl)-2-hydroxy-4-methyl-2-trifluoromethylpentyl)amino]-(1H-quinolin-4-one); 5-[(3,3,3-trifluoro-2-(6-fluoro-4-methylchroman-4-ylmethyl)-2-hydroxypropyl)amino]-(1H-quinolin-3-one); 5-[(4-{3-[1-(benzyloxyimino)ethyl]phenyl}-2-hydroxy-4-methyl-2-trifluoromethylpentyl)amino]-(1H-quinolin-3-one); 6-[(4-(5-acetyl-2-methoxyphenyl)-2-hydroxy-4-methyl-2-trifluoromethylpentyl)amino]-(1H-quinolin-4-one); 7-[(2-hydroxy-4-{3-[1-(methoxyimino)ethyl]phenyl}-4-methyl-2-trifluoromethylpentyl)amino]-(1H-quinolin-4-one); 6-[(4-(5-bromo-2-hydroxyphenyl)-2-hydroxy-4-methyl-2-trifluoromethylpentyl)amino]-(1H-quinolin-4-one); 6-[(2-hydroxy-4-{3-[1-(hydroxyimino)ethyl]phenyl}-4-methyl-2-trifluoromethylpentyl)amino]-(1H-quinolin-4-one); 6-[(4-(5-bromo-2-methoxyphenyl)-2-hydroxy-4-methyl-2-trifluoromethylpentyl)amino]-(1H-quinolin-4-one); 7-[(4-(3,5-difluorophenyl)-2-hydroxy-4-methyl-2-trifluoromethylpentyl)amino]-(1H-quinolin-4-one); 6-[(4-(3,5-dimethylphenyl)-2-hydroxy-4-methyl-2-trifluoromethylpentyl)amino]-(1H-quinolin-4-one); 6-[{2-hydroxy-4-methyl-4-[3-(2-methyl-[1,3]dioxolan-2-yl)phenyl]-2-trifluoromethylpentyl}amino]-(1H-quinolin-4-one); 7-[(4-(2,3-dihydrobenzofuran-7-yl)-2-hydroxy-4-methyl-2-trifluoromethylpentyl)amino]-(1H-[1,5]naphthyridin-4-one); 6-[(4-(3-[1,3]dioxan-2-ylphenyl)-2-hydroxy-4-methyl-2-trifluoromethylpentyl)amino]-(1H-quinolin-4-one); 6-[{4-[3-(3,5-dimethylisoxazol-4-yl)phenyl]-2-hydroxy-4-methyl-2-trifluoromethylpentyl}amino]-(1H-quinolin-4-one); 1-[(4-(2,3-dihydrobenzofuran-7-yl)-2-hydroxy-4-methyl-2-trifluoromethylpentyl)amino]-(3,5-dimethyl-1H-pyridin-4-one); 1-[(4-(5-fluoro-2-methoxyphenyl)-2-hydroxy-4-methyl-2-trifluoromethylpentyl)amino]-(2-hydroxymethyl-3,5-dimethyl-1H-pyridin-4-one); 6-[(4-(5-fluoro-2-hydroxyphenyl)-2-hydroxy-4-methyl-2-tri fluoromethylpentyl) amino]-(3-hydroxymethyl-1H-quinolin-4-one); 6-[(4-(3-bromophenyl)-2-hydroxy-4-methyl-2-trifluoromethylpentyl)amino]-(1H-quinolin-4-one); 6-[(4-(5-fluoro-2-methoxyphenyl)-2-hydroxy-4-methyl-2-trifluoromethylpentyl]-6-methyl-(1H-quinolin-4-one); 6-[(2-hydroxy-4-(2-hydroxy-5-pyridin-3-ylphenyl)-4-methyl-2-trifluoromethylpentyl)amino]-(1H-quinolin-4-one); and 7-[(2-hydroxy-4-(2-hydroxy-5-pyridin-5-ylphenyl)-4-methyl-2-trifluoromethylpentyl)amino]-(1H-quinolin-4-one).

In still another embodiment, said DIGRA compound has Formula Ia or Ib, wherein A, R¹, R², B, D, E, and Q have the meanings disclosed immediately above, and R³ is hydrogen, C₁-C₈ alkyl, C₂-C₈ alkenyl, C₂-C₈ alkynyl, carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein R³ cannot be trifluoromethyl.

In still another embodiment, the present invention provide a method for producing a stereoisomeric DIGRA compound having Formula Ia or Ib, wherein

(a) A is an aryl, heteroaryl, or C₅-C₁₅ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ Spiro cycloalkyl ring;

(c) R³ is the trifluoromethyl group;

(d) B is a methylene group;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an optionally substituted phenyl group having the formula

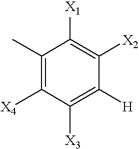

wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein the nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

Non-limiting examples of these compounds include 1-[4-(5-fluoro-2-hydroxyphenyl)-2-hydroxy-4-methyl-2-trifluoromethyl-pentylamino]-(3,5-dichlorobenzene); 1-[4-(5-fluoro-2-hydroxyphenyl)-2-hydroxy-4-methyl-2-trifluoromethyl-pentylamino]-(3-chlorobenzene); 5-(5-fluoro-2-hydroxyphenyl)-3-hydroxy-5-methyl-3-trifluoromethyl-hexanoic acid-(2-chlorophenyl)amide; 5-(5-fluoro-2-hydroxyphenyl)-3-hydroxy-5-methyl-3-trifluoromethyl-hexanoic acid-(2,6-dichloropyrimidin-4-yl)amide; 5-(5-fluoro-2-hydroxyphenyl)-3-hydroxy-5-methyl-3-trifluoromethyl-hexanoic acid-(2,6-dichloropyridin-4-yl)amide; 5-(5-fluoro-2-hydroxyphenyl)-3-hydroxy-5-methyl-3-trifluoromethyl-hexanoic acid-(2,3-dichlorophenyl)amide; 5-(5-fluoro-2-hydroxyphenyl)-2-hydroxy-4-methyl-2-trifluoromethyl-hexanoic acid-(3,5-dimethylphenyl)amide; 5-(5-fluoro-2-hydroxyphenyl)-3-hydroxy-5-methyl-3-trifluoromethyl-hexanoic acid-(3,5-bis-trifluoromethylphenyl)amide; 5-(5-fluoro-2-hydroxyphenyl)-3-hydroxy-5-methyl-3-trifluoromethyl-hexanoic acid-(2,5-dichlorophenyl)amide; 5-(5-fluoro-2-hydroxyphenyl)-3-hydroxy-5-methyl-3-trifluoromethyl-hexanoic acid-(3-bromophenyl)amide; 5-(5-fluoro-2-hydroxyphenyl)-3-hydroxy-5-methyl-3-trifluoromethyl-hexanoic acid-(3,5-difluorophenyl)amide; 5-(5-fluoro-2-hydroxyphenyl)-3-hydroxy-5-methyl-3-trifluoromethyl-hexanoic acid-(3,5-dibromophenyl)-amide.

In still another embodiment, the present invention provides a method for producing a DIGRA compound having Formula Ia or Ib, wherein:

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is C₁-C₈ alkyl, C₂-C₈ alkenyl, C₂-C₈ alkynyl, carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein R³ cannot be trifluoromethyl;

(d) B is a methylene or substituted methylene group, wherein one or two substituent groups of B is independently C₁-C₅ alkyl (or alternatively, C₁-C₃ alkyl), hydroxy, halogen, amino, or oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an azaindolyl group optionally independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from C₁-C₃ alkyl, C₁-C₃ alkoxy, halogen, hydroxy, oxo, cyano, amino, or trifluoromethyl.

Non-limiting examples of these compounds include [1,1,1-trifluoro-4-(5-fluoro-2-methoxyphen-1-yl)-4-methyl-2-(1H-pyrrolo[2,3-c]pyridin-2-ylmethyl)amino]pentan-2-ol; [1,1,1-trifluoro-4-(5-fluoro-2-methoxyphenyl)-4-methyl-2-(1H-pyrrolo[2,3-b]pyridin-2-ylmethyl)amino]pentan-2-ol; [1,1,1-trifluoro-4-(5-fluoro-2-methoxyphenyl)-4-methyl-2-(1H-pyrrolo[3,2-c]pyridin-2-ylmethyl)amino]pentan-2-ol; [1,1,1-trifluoro-4-(5-fluoro-2-methoxyphenyl)-4-methyl-2-(1H-pyrrolo[3,2-b]pyridin-2-ylmethyl)amino]pentan-2-ol; [1,1,1-trifluoro-4-(3-fluorophenyl)-4-methyl-2-(1H-pyrrolo[2,3-c]pyridin-2-ylmethyl)amino]pentan-2-ol; [1,1,1-trifluoro-4-(4-fluorophenyl)-4-methyl-2-(1H-pyrrolo[2,3-c]pyridin-2-ylmethyl)amino]pentan-2-ol; [4-(2,3-dihydrobenzofuran-7-yl)-1,1,1-trifluoro-4-methyl-2-(1H-pyrrolo[2,3-c]pyridin-2-ylmethyl)amino]pentan-2-ol; [4-(2,3-dihydrobenzofuran-7-yl)-1,1,1-trifluoro-4-methyl-2-(1H-pyrrolo[3,2-c]pyridin-2-ylmethyl)amino]pentan-2-ol; [1,1,1-trifluoro-4-methyl-4-phenyl-2-(1H-pyrrolo[2,3-c]pyridine-2-ylmethyl)amino]pentan-2-ol; [1,1,1-trifluoro-4-(4-fluoro-2-methoxyphenyl)-4-methyl-2-(1H-pyrrolo[2,3-c]pyridin-2-ylmethyl)amino]pentan-2-ol; [1,1,1-trifluoro-4-(4-fluoro-2-methoxyphenyl)-4-methyl-2-(1H-pyrrolo[3,2-c]pyridin-2-ylmethyl)amino]pentan-2-ol; [1,1,1-trifluoro-4-methyl-4-phenyl-2-(1H-pyrrolo[3,2-c]pyridin-2-ylmethyl)amino]pentan-2-ol; [1,1,1-trifluoro-4-(4-fluorophenyl)-4-methyl-2-(1H-pyrrolo[3,2-c]pyridin-2-ylmethyl)amino]pentan-2-ol; [1,1,1-trifluoro-4-(5-fluoro-2-methylphenyl)-4-methyl-2-(1H-pyrrolo[2,3-c]pyridin-2-ylmethyl)amino]pentan-2-ol; [1,1,1-trifluoro-4-(5-fluoro-2-methoxyphenyl)-4-methyl-2-(3-methyl-1H-pyrrolo[2,3-c]pyridin-2-ylmethyl)amino]pentan-2-ol; and [1,1,1-trifluoro-4-(5-fluoro-2,3-dihydrobenzofuran-7-yl)-4-methyl-2-(1H-pyrrolo[2,3-c]pyridine-2-ylmethyl)amino]pentan-2-ol.

In still another embodiment, the present invention provides a method for producing a DIGRA compound having Formula Ia or Ib, wherein

(a) A is cycloalkyl, an aryl, or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ Spiro cycloalkyl ring;

(c) R³ is the trifluoromethyl group;

(d) B is a methylene or substituted methylene group having one or two substituents independently selected from the group consisting of C₁-C₃ alkyl, hydroxy, halogen, amino, and oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

Non-limiting examples of these compounds include 4-cyclohexyl-1,1,1-trifluoro-4-methyl-2-[(2-methyl-quinolin-4-yl)amino]pentan-2-ol; 1,1,1-trifluoro-4-(5-fluoro-2-methoxyphen-1-yl)-4-methyl-2-[(3-methyl-1H-pyrrolo[3,2-c]pyridin-2-ylmethyl)amino]pentan-2-ol; 1,1,1-trifluoro-4-(5-fluoro-2,3-dihydrobenzofuran-7-yl)-4-methyl-2-[(1H-pyrrolo[3,2-c]pyridin-2-ylmethyl)amino]pentan-2-ol; 1,1,1-trifluoro-4-(5-fluoro-2-methylphen-1-yl)-4-methyl-2-[(3-methyl-1H-pyrrolo[2,3-c]pyridin-2-ylmethyl)amino]pentan-2-ol; 2-[(4,6-dimethyl-1H-pyrrolo[3,2-c]pyridin-2-ylmethyl)amino]-1,1,1-trifluoro-4-(5-fluoro-2-methoxyphenyl)-4-methylpentan-2-ol; 2-[(5,7-dimethyl-1H-pyrrolo[2,3-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(6-methyl-1H-pyrrolo[3,2-c]pyridin-2-ylmethyl)amino]pentan-2-ol; 1,1,1-trifluoro-4-(5-fluoro-2-methoxyphenyl)-4-methyl-2-[(4-methyl-1H-pyrrolo[3,2-c]pyridin-2-ylmethyl)amino]pentan-2-ol; 1,1,1-trifluoro-4-(5-fluoro-2-methoxyphenyl)-4-methyl-2-[(5H-pyrrolo[3,2-d]pyrimidin-6-ylmethyl)amino]pentan-2-ol; 1,1,1-trifluoro-4-(5-fluoro-2-methoxyphenyl)-4-methyl-2-[(thieno[2,3-d]pyridazin-2-ylmethyl)amino]pentan-2-ol; 1,1,1-trifluoro-4-(5-fluoro-2-methoxyphenyl)-4-methyl-2-[(5H-pyrrolo[3,2-c]pyridazin-6-ylmethyl)amino]pentan-2-ol; 1,1,1-trifluoro-4-(5-fluoro-2-methoxyphenyl)-4-methyl-2-[(2-methyl-5H-pyrrolo[3,2-d]pyrimidin-6-ylmethyl)amino]pentan-2-ol; 1,1,1-trifluoro-4-(5-fluoro-2-methylphenyl)-4-methyl-2-[(1H-pyrrolo[2,3-d]pyridazin-2-ylmethyl)amino]pentan-2-ol; 2-[(4,6-dimethyl-H-pyrrolo[3,2-c]pyridin-2-ylmethyl)amino]-1,1,1-trifluoro-4-(5-fluoro-2-methylphenyl)-4-methylpentan-2-ol; 4-(5-chloro-2,3-dihydrobenzofuran-7-yl)-2-[(4,6-dimethyl-1H-pyrrolo[3,2-c]pyridin-2-ylmethyl)amino]-1,1,1-trifluoro-4-methylpentan-2-ol; 4-(5-chloro-2,3-dihydrobenzofuran-7-yl)-1,1,1-trifluoro-4-methyl-2-[(3-methyl-1H-pyrrolo[2,3-c]pyridin-2-ylmethyl)amino]pentan-2-ol; 1,1,1-trifluoro-4-(5-fluoro-2-methylphenyl)-4-methyl-2-[(5H-pyrrolo[3,2-c]-pyridazin-6-ylmethyl)amino]pentan-2-ol; 4-(5-chloro-2,3-dihydrobenzofuran-7-yl)-1,1,1-trifluoro-4-methyl-2-[(5H-pyrrolo[3,2-c]pyridazin-6-ylmethyl)amino]pentan-2-ol; 4-(5-chloro-2,3-dihydrobenzofuran-7-yl)-1,1,1-trifluoro-4-methyl-2-[(1H-pyrrolo[2,3-d]pyridazin-2-ylmethyl)amino]pentan-2-ol; 1,1,1-trifluoro-4-(5-fluoro-2-methoxyphenyl)-2-[(7-fluoro-1H-pyrrolo[2,3-c]pyridin-2ylmethyl)amino]-4-methylpentan-2-ol; 1,1,1-trifluoro-4-(5-fluoro-2-methoxyphenyl)-4-methyl-2-[(4-methyl-1H-pyrrolo[2,3-c]pyridin-2-ylmethyl)amino]pentan-2-ol; 2-[(5,7-dichloro-1H-pyrrolo[2,3-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(5-trifluoromethyl-1H-pyrrolo[2,3-c]pyridin-2-ylmethyl)amino]pentan-2-ol; 1,1,1-trifluoro-4-(5-fluoro-2-methoxyphenyl)-2-[(5-methoxy-1H-pyrrolo[2,3-c]pyridin-2-ylmethyl)amino]-4-methylpentan-2-ol; 1,1,1-trifluoro-4-(5-fluoro-2-methylphenyl)-4-methyl-2-[(4-methyl-1H-pyrrolo[2,3-c]pyridin-2-ylmethyl)amino]pentan-2-ol; 1,1,1-trifluoro-4-(5-fluoro-2-methylphenyl)-2-[(5-isopropoxy-1H-pyrrolo[2,3-c]pyridin-2-ylmethyl)amino]-4-methylpentan-2-ol; 1,1,1-trifluoro-4-(5-fluoro-2-methylphenyl)-2-[(5-methoxy-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-2-[(5-methoxy-1H-pyrrolo[2,3-c]pyridin-2-ylmethyl)amino]-4-methylpentan-2-ol; 1,1,1-trifluoro-4-(5-fluoro-2-methylphenyl)-2-[(7-fluoro-1H-pyrrolo[2,3-c]pyridin-2-ylmethyl)amino]-4-methylpentan-2-ol; 4-(5-chloro-2,3-dihydrobenzofuran-7-yl)-1-trifluoro-4-methyl-2-[(5-trifluoromethyl-1H-pyrrolo[2,3-c]pyridin-2-ylmethyl)amino]pentan-2-ol; 1,1,1-trifluoro-4-(5-fluoro-2-methylphenyl)-4-methyl-2-[(5-trifluoromethyl-1H-pyrrolo[2,3-c]pyridin-2-ylmethyl)amino]pentan-2-ol; 4-(5-chloro-2,3-dihydrobenzofuran-7-yl)-1,1,1-trifluoro-2-[(5-isopropoxy-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-2-[(7-fluoro-1H-pyrrolo[2,3-c]pyridin-2-ylmethyl)amino]-4-methylpentan-2-ol; 4-(5-chloro-2,3-dihydrobenzofuran-7-yl)-2-[(5-dimethylamino-1H-pyrrolo[2,3-c]pyridin-2-ylmethyl)amino]-1,1,1-trifluoro-4-methylpentan-2-ol; 4-(5-chloro-2,3-dihydrobenzofuran-7-yl)-1,1,1-trifluoro-4-methyl-2-[(5-piperidin-1-yl-1H-pyrrolo[2,3-c]pyridin-2-ylmethyl)amino]pentan-2-ol; 4-(5-chloro-2,3-dihydrobenzofuran-7-yl)-1,1-trifluoro-4-methyl-2-[(5-morpholin-4-yl-1H-pyrrolo[2,3-c]pyridin-2-ylmethyl)amino]pentan-2-ol; 1,1,1-trifluoro-4-(5-fluoro-2-methylphenyl)-4-methyl-2-[(5-piperidin-1-yl-1H-pyrrolo[2,3-c]pyridin-2-ylmethyl)amino]pentan-2-ol; 4-(5-chloro-2,3-dihydrobenzofuran-7-yl)-2-[(5-ethoxy-1H-pyrrolo[2,3-c]pyridin-2-ylmethyl)amino]-1,1,1-trifluoro-4-methylpentan-2-ol; 2-[(5-benzyloxy-1H-pyrrolo[2,3-c]pyridin-2-ylmethyl)amino]-1,1,1-trifluoro-4-(5-fluoro-2-methylphenyl)-4-methylpentan-2-ol; 2-[(5-benzyloxy-1H-pyrrolo[2,3-c]pyridin-2-ylmethyl)amino]-4-(5-chloro-2,3-dihydrobenzofuran-7-yl)-1,1,1-trifluoro-4-methylpentan-2-ol; 1,1,1-trifluoro-4-(5-fluoro-2-methoxyphenyl)-2-[(5-chloro-1H-pyrrolo[2,3-c-]pyridin-2-ylmethyl)amino]-4-methylpentan-2-ol; 1,1,1-trifluoro-4-(5-fluoro-2-methoxyphenyl)-4-methyl-2-[(5-(methylamino)-1H-pyrrolo[2,3-c]pyridin-2-ylmethyl)amino]pentan-2-ol; 1,1,1-trifluoro-4-(5-fluoro-2-methoxyphenyl)-4-methyl-2-[(5-amino-1H-pyrrolo[2,3-c]pyridin-2-ylmethyl)amino]pentan-2-ol; 1,1,1-trifluoro-4-(5-fluoro-2-methylphenyl)-4-methyl-2-[(6-amino-1H-pyrrol-o[2,3-c]pyridin-2-ylmethyl)amino]pentan-2-ol; 4-(5-chloro-2,3-dihydrobenzofuran-7-yl)-1,1,1-trifluoro-2-[(5-amino-1H-pyrrolo[2,3-c]pyridin-2-ylmethyl)amino]-4-methylpentan-2-ol; 4-(5-chloro-2,3-dihydrobenzofuran-7-yl)-1,1,1-trifluoro-4-methyl-2-[(5-methylamino-1H-pyrrolo[2,3-c]pyridin-2-ylmethyl)amino]pentan-2-ol; 4-(5-bromo-2,3-dihydrobenzofuran-7-yl)-1,1,1-trifluoro-4-methyl-2-[(1H-pyrrolo[2,3-c]pyridin-2-ylmethyl)amino]pentan-2-ol; 1,1,1-trifluoro-4-methyl-4-(5-methyl-2,3-dihydrobenzofuran-7-yl)-2-[(1H-pyrrolo[2,3-c]pyridin-2-ylmethyl)amino]pentan-2-ol; 4-(5-chloro-2,3-dihydrobenzofuran-7-yl)-1,1,1-trifluoro-4-methyl-2-[(1H-pyrrolo[2,3-c]pyridin-2-ylmethyl)amino]pentan-2-ol; 1,1,1-trifluoro-4-(5-fluoro-2-methoxyphenyl)-4-methyl-2-[(pyrrolo[2,3-b]pyridin-1-ylmethyl)amino]pentan-2-ol; 1,1,1-trifluoro-4-(5-fluoro-2-methoxyphenyl)-4-methyl-2-[(6-oxy-1H-pyrrolo[2,3-c]pyridin-2-ylmethyl)amino]pentan-2-ol; 1,1,1-trifluoro-4-(5-fluoro-2-methoxyphenyl)-4-methyl-2-[(pyrrolo[2,3-c]pyridin-1-ylmethyl)amino]pentan-2-ol; 2-[(benzo[b]thiophen-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-4-methyl-2-[(thieno[2,3-c]pyridin-2-ylmethyl)amino]pentan-2-ol; 1,1,1-trifluoro-4-(5-fluoro-2-methoxyphenyl)-2-[(indazol-1-ylmethyl)amino]-4-methylpentan-2-ol; 1,1,1-trifluoro-4-(5-fluoro-2-methoxyphenyl)-4-methyl-2-[(pyrazolo[1,5-a]pyridin-2-ylmethyl)amino]pentan-2-ol; 1,1,1-trifluoro-4-(5-fluoro-2-methoxyphenyl)-2-[(furo[2,3-c]pyridin-2-ylmethyl)amino]-4-methylpentan-2-ol; 1,1,1-trifluoro-4-methyl-4-(5-methyl-2,3-dihydrobenzofuran-7-yl)-2-[(1H-pyrrolo[3,2-c]pyridin-2-ylmethyl)amino]pentan-2-ol; 4-(5-chloro-2,3-dihydrobenzofuran-7-yl)-1,1,1-trifluoro-4-methyl-2-[(1H-pyrrolo[3,2-c]pyridin-2-ylmethyl)amino]pentan-2-ol; 4-(5-bromo-2,3-dihydrobenzofuran-7-yl)-1,1,1-trifluoro-4-methyl-2-[(1H-pyrrolo[3,2-c]pyridin-2-ylmethyl)amino]pentan-2-ol; 2-[(3-dimethylaminomethyl-1H-pyrrolo[3,2-c]pyridin-2-ylmethyl)amino]-1,1,1-trifluoro-4-(5-fluoro-2-methoxyphenyl)-4-methylpentan-2-ol; 1,1,1-trifluoro-4-(5-fluoro-2-methoxyphenyl)-2-[(furo[3,2-c]pyridin-2-ylmethyl)amino]-4-methylpentan-2-ol; 4-(5-chloro-2,3-dihydrobenzofuran-7-yl)-1,1,1-trifluoro-4-methyl-2-[(pyrrolo[3,2-b]pyridin-1-ylmethyl)amino]pentan-2-ol; 1,1,1-trifluoro-4-(5-fluoro-2-methoxyphenyl)-4-methyl-2-[(thieno[3,2-c]pyridin-2-ylmethyl)amino]pentan-2-ol; 4-(5-chloro-2,3-dihydrobenzofuran-7-yl)-1,1,1-trifluoro-4-methyl-2-[(thieno[3,2-c]pyridin-2-ylmethyl)amino]pentan-2-ol; 1,1,1-trifluoro-4-(5-fluoro-2-methylphenyl)-4-methyl-2-[(pyrrolo[3,2-b]pyridin-1-ylmethyl)amino]pentan-2-ol; 1,1,1-trifluoro-4-(5-fluoro-2-methylphenyl)-4-methyl-2-[(thieno[3,2-c]pyridin-2-ylmethyl)amino]pentan-2-ol; 2-[4-(5-fluoro-2-hydroxyphenyl)-2-hydroxy-4-methyl-2-trifluoromethylpentylamino]-H-indole-6-carboxylic acid; 2-[4-(5-fluoro-2-hydroxyphenyl)-2-hydroxy-4-methyl-2-trifluoromethylpentylamino]-1H-indole-6-carboxylic acid dimethylamide; 2-[4-(5-fluoro-2-methoxyphenyl)-2-hydroxy-4-methyl-2-trifluoromethylpentylamino]-1H-indole-6-carboxylic acid dimethylamide; 2-[4-(5-fluoro-2-hydroxyphenyl)-2-hydroxy-4-methyl-2-trifluoromethylpentylamino]-1H-indole-6-carboxylic acid amide; 2-[4-(5-fluoro-2-methoxyphenyl)-2-hydroxy-4-methyl-2-trifluoromethylpentylamino]-1H-indole-6-carboxylic acid amide; 1,1,1-trifluoro-4-(4-fluoro-2-methoxyphenyl)-2-[(7-fluoro-4-methyl-1H-indol-2-ylmethyl)amino]-4-methylpentan-2-ol; 2-[4-(5-fluoro-2-methoxyphenyl)-2-hydroxy-4-methyl-2-trifluoromethylpentylamino]-1H-indole-5-carboxylic acid-2-trimethylsilanylethyl ester; 2-[4-(5-fluoro-2-methoxyphenyl)-2-hydroxy-4-methyl-2-trifluoromethylpentylamino]-1H-indole-5-carboxylic acid; 2-[4-(5-fluoro-2-methoxyphenyl)-2-hydroxy-4-methyl-2-trifluoromethylpentylamino]-1H-indole-5-carboxylic acid methylamide; 2-[4-(5-bromo-2,3-dihydrobenzofuran-7-yl)-2-hydroxy-4-methyl-2-trifluoromethylpentylamino]-1H-indole-5-carboxylic acid; 2-[4-(5-bromo-2,3-dihydrobenzofuran-7-yl)-2-hydroxy-4-methyl-2-trifluoromethylpentylamino]-1H-indole-5-carboxylic acid amide; 2-[4-(5-bromo-2,3-dihydrobenzofuran-7-yl)-2-hydroxy-4-methyl-2-trifluoromethylpentylamino]-1H-indole-5-carboxylic acid dimethylamide; 2-[4-(5-bromo-2,3-dihydrobenzofuran-7-yl)-2-hydroxy-4-methyl-2-trifluoromethylpentylamino]-1H-indole-5-carboxylic acid cyanomethylamide; 2-[4-(5-chloro-2,3-dihydrobenzofuran-7-yl)-2-hydroxy-4-methyl-2-trifluoromethylpentylamino]-5-methyl-1,5-dihydropyrrolo[3,2-c]pyridin-6-one; 4-(5-chloro-2,3-dihydrobenzofuran-7-yl)-1,1,1-trifluoro-2-[(6-methoxy-5,6-dihydro-1H-pyrrolo[3,2-c]pyridin-2-ylmethyl)amino]-4-methylpentan-2-ol; 2-[4-(5-chloro-2,3-dihydrobenzofuran-7-yl)-2-hydroxy-4-methyl-2-trifluoromethylpentylamino]-1,7-dihydropyrrolo[3,2-c]pyridine-4,6-dione; and 6-[4-(5-chloro-2,3-dihydrobenzofuran-7-yl)-2-hydroxy-4-methyl-2-trifluoromethylpentylamino]-3-methyl-1,7-dihydropyrrolo[2,3-d]pyrimidine-2,4-dione.

In still another embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is hydrogen, C₁-C₈ alkyl, C₂-C₈ alkenyl, C₂-C₈ alkynyl, carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone, wherein R³ cannot be trifluoromethyl;

(d) B is a methylene or substituted methylene group having one or two substituent groups independently selected from the group consisting of C₁-C₃ alkyl, hydroxy, halogen, amino, and oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₁ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

Non-limiting examples of these compounds include 2-cyclopropyl-4-(5-fluoro-2-methoxyphenyl)-4-methyl-1-[(1H-pyrrolo[3,2-c]pyridin-2-yl)amino]pentan-2-ol; 2-cyclopropyl-4-(5-fluoro-2-methylphenyl)-4-methyl-1-[(1H-pyrrolo[2,3-c]pyridin-2-yl)amino]pentan-2-ol; 4-(5-chloro-2,3-dihydrobenzofuran-7-yl)-2-cyclopropyl-4-methyl-1-[(1H-pyrrolo[2,3-c]pyridin-2-yl)amino]pentan-2-ol; 2-cyclopropyl-4-(5-fluoro-2-methylphenyl)-4-methyl-1-[(1H-pyrrolo[3,2-c]pyridin-2-yl)amino]pentan-2-ol; 4-(5-chloro-2,3-dihydrobenzofuran-7-yl)-2-cyclopropyl-4-methyl-1-[(1H-pyrrolo[3,2-c]pyridin-2-yl)amino]pentan-2-ol; 4-(5-fluoro-2-methoxyphenyl)-2,4-dimethyl-1-[(1H-pyrrolo[2,3-c]pyridin-2-yl)amino]pentan-2-ol; 2-cyclohexyl-4-(5-fluoro-2-methoxyphenyl)-4-methyl-1-[(1H-pyrrolo[2,3-c]pyridin-2-yl)amino]pentan-2-ol; 2-cyclopentyl-4-(5-fluoro-2-methoxyphenyl)-4-methyl-1-[(1H-pyrrolo[2,3-c]pyridin-2-yl)amino]pentan-2-ol; 1,1-difluoro-4-(5-fluoro-2-methoxyphenyl)-4-methyl-2-[(1H-pyrrolo[2,3-c]pyridin-2-ylmethyl)amino]pentan-2-ol; 2-cyclobutyl-4-(5-fluoro-2-methoxyphenyl)-4-methyl-1-[(1H-pyrrolo[2,3-c]pyridin-2-yl)amino]pentan-2-ol; 1-fluoro-4-(5-fluoro-2-methoxyphenyl)-4-methyl-2-[(1H-pyrrolo[2,3-c]pyridin-2-ylmethyl)amino]pentan-2-ol; 4-(5-fluoro-2-methoxyphenyl)-4-methyl-2-phenyl-1-[(1H-pyrrolo[2,3-c]pyridin-2-yl)amino]pentan-2-ol; 1,1-difluoro-4-(5-fluoro-2-methoxyphenyl)-4-methyl-2-[(1H-pyrrolo[3,2-c]pyridin-2-ylmethyl)amino]pentan-2-ol; 2-(1-fluorocyclopropyl)-4-(5-fluoro-2-methoxyphenyl)-4-methyl-1-[(1H-pyrrolo[2,3-c]pyridin-2-yl)amino]pentan-2-ol; 2-(1-fluorocyclopropyl)-4-(4-fluorophenyl)-4-methyl-(1-quinolin-4-ylamino)pentan-2-ol; 4-(5-chloro-2,3-dihydrobenzofuran-7-yl)-1,1-difluoro-4-methyl-2-[(1H-pyrrolo[3,2-c]pyridin-2-ylmethyl)amino]pentan-2-ol; 4-(5-chloro-2,3-dihydrobenzofuran-7-yl)-1,1-difluoro-4-methyl-2-[(pyrrolo[3,2-b]pyridin-1-ylmethyl)amino]pentan-2-ol; 4-(5-fluoro-2-methylphenyl)-2,4-dimethyl-1-[(5-phenyl-1H-pyrrolo[2,3-c]pyridin-2-yl)amino]pentan-2-ol; 4-(5-chloro-2,3-dihydrobenzofuran-7-yl)-1,1-difluoro-4-methyl-2-[(6-methyl-1H-pyrrolo[3,2-c]pyridin-2-ylmethyl)amino]pentan-2-ol; 4-(5-chloro-2,3-dihydrobenzofuran-7-yl)-2,4-dimethyl-1-[(5-phenyl-1H-pyrrolo[2,3-c]pyridin-2-yl)amino]pentan-2-ol; 1,1-difluoro-4-(5-methanesulfonyl-2,3-dihydrobenzofuran-7-yl)-4-methyl-2-[(1H-pyrrolo[2,3-c]pyridin-2-ylmethyl)amino]pentan-2-ol; and 2-(5-bromo-1H-indol-2-ylmethyl)amino-1,1-difluoro-4-(5-methanesulfonyl-2,3-dihydrobenzofuran-7-yl)-4-methylpentan-2-ol.

In still another embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently C₁-C₅ alkyl, wherein one or both are independently substituted with hydroxy, C₁-C₅ alkoxy, C₁-C₅ alkylthio wherein the sulfur atom is optionally oxidized to a sulfoxide or sulfone, amino wherein the nitrogen atom is optionally independently mono- or di-substituted by C₁-C₅ alkyl or aryl;

(c) R³ is hydrogen, C₁-C₈ alkyl (preferably C₁-C₅ alkyl, more preferably C₁-C₃ alkyl), C₂-C₈ alkenyl (preferably C₁-C₅ alkenyl, more preferably C₁-C₃ alkenyl), C₂-C₈ alkynyl (preferably C₁-C₅ alkynyl, more preferably C₁-C₃ alkynyl), carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₅ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₅ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone;

(d) B is methylene;

(e) D is —NH— group;

(f) E is the hydroxy group; and

(g) Q comprises a heteroaryl group optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, acyl, C₁-C₃ silanyloxy, C₁-C₅ alkoxycarbonyl, carboxy, halogen, hydroxy, oxo, cyano, heteroaryl, heterocyclyl, amino wherein the nitrogen atom is optionally independently mono- or di-substituted by C₁-C₅ alkyl or aryl, ureido wherein either nitrogen atom is optionally independently substituted with C₁-C₅ alkyl, or trifluoromethyl.

In yet another embodiment, the present invention provides a method for producing a DIGRA compound having Formula Ia or Ib, wherein

(a) A is an aryl, heteroaryl, heterocyclyl, or C₃-C₈ cycloalkyl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl;

(c) R³ is the trifluoromethyl group;

(d) B is C₁-C₅ alkylene, C₂-C₅ alkenylene, or C₂-C₅ alkynylene, each optionally independently substituted with one to three substituent groups, wherein each substituent group of B is independently C₁-C₃ alkyl, hydroxy, halogen, amino, or oxo;

(e) D is —NH— or —NR′—, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises an indolyl group optionally substituted with one to three substituent groups, wherein each substituent group of Q is independently C₁-C₅ alkyl, C₂-C₅ alkenyl, C₂-C₅ alkynyl, C₃-C₅ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is optionally independently substituted with one to three substituent groups selected from the group consisting of C₁-C₃ alkyl, C₁-C₃ alkoxy, halogen, hydroxy, oxo, cyano, amino, and trifluoromethyl.

Non-limiting examples of these compounds include 4-(5-bromo-2,3-dihydrobenzofuran-7-yl)-1,1,1-trifluoro-2-[(1H-indol-2-ylmethyl)amino]-4-methylpentan-2-ol; 1,1,1-trifluoro-2-(1H-indol-2-ylmethylamino)-4-methyl-4-pyridin-2-ylpentan-2-ol; 4-(2,3-dihydro-5-cyanobenzofuran-7-yl)-1,1,1-trifluoro-2-((1H-indol-2-yl-methyl)amino)]-4-methylpentan-2-ol; 4-(2,3-dihydrobenzofuran-7-yl)-1,1,1-trifluoro-2-[(1H-indol-2-ylmethyl)amino]-4-methylpentan-2-ol; 1,1,1-trifluoro-4-(5-fluoro-2,3-dihydrobenzofuran-7-yl)-2-[(1H-indol-2-ylmethyl)amino]-4-methylpentan-2-ol; 1,1,1-trifluoro-2-[(1H-indol-2-ylmethyl)amino]-4-methyl-4-(5-methyl-2,3-dihydrobenzofuran-7-yl)pentan-2-ol; 4-(2,3-dihydrobenzofuran-5-yl)-1,1,1-trifluoro-2-[(1H-indol-2-ylmethyl)amino]-4-methylpentan-2-ol; 4-(2,3-dihydrobenzofuran-7-yl)-1,1,1-trifluoro-4-methyl-2-[(5-trifluoromethyl-1H-indol-2-ylmethyl)amino]pentan-2-ol; and 1,1,1-trifluoro-2-[(1H-indol-2-ylmethyl)amino]-4-methyl-4-thiophen-3-ylpentan-2-ol.

In a further embodiment, the present invention provides a method for producing a DIGRA compound having Formula Ia or Ib, wherein

(a) A is an aryl or heteroaryl group, each optionally independently substituted with one to three substituent groups, which are independently selected from the group consisting of C₁-C₅ alkyl, C₂-C₅ alkenyl, C₂-C₅ alkynyl, C₁-C₃ alkanoyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, amino wherein the nitrogen atom is optionally independently mono- or di-substituted by C₁-C₅ alkyl or aryl, ureido wherein either nitrogen atom is optionally independently substituted with C₁-C₅ alkyl, C₁-C₅ alkylthio wherein the sulfur atom is optionally oxidized to a sulfoxide or sulfone;

(b) R¹ and R² are each independently hydrogen or C₁-C₅ alkyl, or R¹ and R² together with the carbon atom they are commonly attached to form a C₃-C₈ spiro cycloalkyl ring;

(c) R³ is carbocycle, heterocyclyl, aryl, heteroaryl, carbocycle-C₁-C₈ alkyl, carboxy, alkoxycarbonyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups, wherein each substituent group of R³ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl, ureido wherein either nitrogen atom is optionally independently substituted with C₁-C₅ alkyl, C₁-C₅ alkylthio wherein the sulfur atom is optionally oxidized to a sulfoxide or sulfone;

(d) B is a methylene or substituted methylene group having one or two substituent groups selected from the group consisting of C₁-C₃ alkyl, hydroxy, halogen, amino, and oxo;

(e) D is an —NH— or —NR′— group, wherein R′ comprises an unsubstituted or substituted C₁-C₁₅ (alternatively, C₁-C₁₀, or C₁-C₅, or C₁-C₃) linear or branched alkyl group;

(f) E is the hydroxy group; and

(g) Q comprises the group

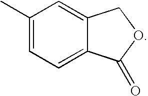

Non-limiting examples of these compounds include 2-benzyl-4-methyl-4-phenyl-(1-oxo-1,3-dihydroisobenzofuran-5-yl)amino-pentan-2-ol; 4-methyl-2,4-diphenyl-(1-oxo-1,3-dihydroisobenzofuran-5-yl)amino-pentan-2-ol; 4-methyl-2-phenethyl-4-phenyl-(1-oxo-1,3-dihydroisobenzofuran-5-yl)amino-pentan-2ol; 2-(3-methoxybenzyl)-4-methyl-4-phenyl-(1-oxo-1,3-dihydroisobenzofuran-5-yl)amino-pentan-2-ol; 2-(4-methoxybenzyl)-4-methyl-4-phenyl-(1-oxo-1,3-dihydroisobenzofuran-5-yl)amino-pentan-2-ol; 2-[2-(4-methoxyphenyl)ethyl]-4-methyl-4-phenyl-(1-oxo-1,3-dihydroisobenzofuran-5-yl)amino-pentan-2-ol; 2-cyclohexylmethyl-4-methyl-4-phenyl-(1-oxo-1,3-dihydroisobenzofuran-5-yl)amino-pentan-2-ol; 2-(4-tert-butylbenzyl)-4-methyl-4-phenyl-(1-oxo-1,3-dihydroisobenzofuran-5-yl)amino-pentan-2-ol; 2-biphenyl-5-ylmethyl-4-methyl-4-phenyl-(1-oxo-1,3-dihydroisobenzofuran-5-yl)amino-pentan-2-ol; 4-methyl-2-naphthalen-2-ylmethyl-4-phenyl-(1-oxo-1,3-dihydroisobenzofuran-5-yl)amino-pentan-2-ol; 2-(3-hydroxybenzyl)-4-methyl-4-phenyl-(1-oxo-1,3-dihydroisobenzofuran-5-yl)amino-pentan-2-ol; 4-methyl-2-(2-methyl-2-phenylpropyl)-4-phenyl-(1-oxo-1,3-dihydroisobenzofuran-5-yl)amino-pentan-2-ol; 2-benzyl-4-(5-fluoro-2-methoxyphenyl)-4-methyl-(1-oxo-1,3-dihydroisobenzofuran-5-yl)amino-pentan-2ol; 2-cyclohexylmethyl-4-(5-fluoro-2-methoxyphenyl)-4-methyl-(1-oxo-1,3-dihydroisobenzofuran-5-yl)amino-pentan-2-ol; 2-benzyl-4-(5-fluoro-2-hydroxyphenyl)-4-methyl-(1-oxo-1,3-dihydroisobenzofuran-5-yl)amino-pentan-2-ol; 2-cyclohexylmethyl-4-(5-fluoro-2-hydroxyphenyl)-4-methyl-(1-oxo-1,3-dihydroisobenzofuran-5-yl)amino-pentan-2-ol; 4-(5-fluoro-2-hydroxyphenyl)-4-methyl-2-(2-methyl-2-phenylpropyl)-(1-oxo-1,3-dihydroisobenzofuran-5-yl)amino-pentan-2ol; 2-(2-chloro-6-fluorobenzyl)-4-(5-fluoro-2-methoxyphenyl)-4-methyl-(1-oxo-1,3-dihydroisobenzofuran-5-yl)amino-pentan-2ol; 2-(3-fluorobenzyl)-4-(5-fluoro-2-methoxyphenyl)-4-methyl-(1-oxo-1,3-dihydroisobenzofuran-5-yl)amino-pentan-2ol; 2-(2-fluorobenzyl)-4-(5-fluoro-2-methoxyphenyl)-4-methyl-(1-oxo-1,3-dihydroisobenzofuran-5-yl)amino-pentan-2ol; 2-(3,4-difluorobenzyl)-4-(5-fluoro-2-methoxyphenyl)-4-methyl-(1-oxo-1,3-dihydroisobenzofuran-5-yl)amino-pentan-2-ol; 2-(2-chloro-6-fluorobenzyl)-4-(5-fluoro-2-hydroxyphenyl)-4-methyl-(1-oxo-1,3-dihydroisobenzofuran-5-yl)amino-pentan-2ol; 2-(3-fluorobenzyl)-4-(5-fluoro-2-hydroxyphenyl)-4-methyl-(1-oxo-1,3-dihydroisobenzofuran-5-yl)amino-pentan-2-ol; 2-(2-fluorobenzyl)-4-(5-fluoro-2-hydroxyphenyl)-4-methyl-(1-oxo-1,3-dihydroisobenzofuran-5-yl)amino-pentan-2ol; 2-(3,4-difluorobenzyl)-4-(5-fluoro-2-hydroxyphenyl)-4-methyl-(1-oxo-1,3-dihydroisobenzofuran-5-yl)amino-pentan-2ol; 2-(4-fluorobenzyl)-4-(5-fluoro-2-methoxyphenyl)-4-methyl-(1-oxo-1,3-dihydroisobenzofuran-5-yl)amino-pentan-2-ol; 4-(5-fluoro-2-methoxyphenyl)-4-methyl-2-(3-methylbenzyl)-(1-oxo-1,3-dihydroisobenzofuran-5-yl)amino-pentan-2-ol; 2-(4-fluorobenzyl)-4-(5-fluoro-2-hydroxyphenyl)-4-methyl-(1-oxo-1,3-dihydroisobenzofuran-5-yl)amino-pentan-2-ol; 4-(5-fluoro-3-hydroxyphenyl)-4-methyl-2-(3-methylbenzyl)-(1-oxo-1,3-dihydroisobenzofuran-5-yl)amino-pentan-2-ol; 2-(3,5-difluorophenyl)-4-(5-fluoro-2-hydroxyphenyl)-4-methyl-(1-oxo-1,3-dihydroisobenzofuran-5-yl)amino-pentan-2-ol; 4-(5-fluoro-2-methoxyphenyl)-4-methyl-2-(2-methylbenzyl)-(1-oxo-1,3-dihydroisobenzofuran-5-yl)amino-pentan-2-ol; 2-(3,5-dimethylbenzyl)-4-(5-fluoro-2-methoxyphenyl)-4-methyl-(1-oxo-1,3-dihydroisobenzofuran-5-yl)amino-pentan-2-ol; 2-(2,5-difluorobenzyl)-4-(5-fluoro-2-methoxyphenyl)-4-methyl-(1-oxo-1,3-dihydroisobenzofuran-5-yl)amino-pentan-2-ol; 2-(2,5-difluorobenzyl)-4-(5-fluoro-2-hydroxyphenyl)-4-methyl-(1-oxo-1,3-dihydroisobenzofuran-5-yl)amino-pentan-2-ol; 4-(5-fluoro-2-hydroxyphenyl)-4-methyl-2-(2-methylbenzyl)-(1-oxo-1,3-dihydroisobenzofuran-5-yl)amino-pentan-2-ol; 2-(3,5-dimethylbenzyl)-4-(5-fluoro-2-hydroxyphenyl)-4-methyl-(1-oxo-1,3-dihydroisobenzofuran-5-yl)amino-pentan-2-ol; 2-(3-chlorobenzyl)-4-(5-fluoro-2-hydroxyphenyl)-4-methyl-(1-oxo-1,3-dihydroisobenzofuran-5-yl)amino-pentan-2-ol; 4-(5-fluoro-2-methoxyphenyl)-2-[2-(4-methoxyphenyl)ethyl]-4-methyl-(1-oxo-1,3-dihydroisobenzofuran-5-yl)amino-pentan-2-ol; and 4-(5-fluoro-2-methoxyphenyl)-2-(2-methoxybenzyl)-4-methyl-(1-oxo-1,3-dihydroisobenzofuran-5-yl)amino-pentan-2-ol. Other non-limiting examples of these compounds include 6-(2-benzyl-2-hydroxy-4-methyl-4-phenylpentylamino)isobenzofuran-1(3H)-one; 5-(2-hydroxy-4-methyl-2,4-diphenylpentylamino)isobenzofuran-1(3H)-one; 5-(2-hydroxy-4-methyl-2-phenethyl-4-phenylpentylamino)isobenzofuran-1(3H)-one; 6-(2-hydroxy-2-(3-methoxybenzyl)-4-methyl-4-phenylpentylamino)isobenzofuran-1(3H)-one; 5-(2-hydroxy-2-(4-methoxybenzyl)-4-methyl-4-phenylpentylamino)isobenzofuran-1(3H)-one; 5-(2-hydroxy-2-[2-(4-methoxyphenyl)ethyl]-4-methyl-4-phenylpentylamino)isobenzofuran-1(3H)-one; 6-(2-cyclohexylmethyl-2-hydroxy-4-methyl-4-phenylpentylamino)isobenzofuran-1(3H)-one; 5-(2-(4-tert-butylbenzyl)-2-hydroxy-4-methyl-4-phenylpentylamino)isobenzofuran-1(3H)-one; 5-(2-biphenyl-4-ylmethyl-2-hydroxy-4-methyl-4-phenylpentylamino)isobenzofuran-1(3H)-one; 5-(2-hydroxy-4-methyl-2-naphthalen-2-ylmethyl-4-phenylpentylamino)isobenzofuran-1(3H)-one; 6-(2-hydroxy-2-(3-hydroxybenzyl)-4-methyl-4-phenylpentylamino)isobenzofuran-1(3H)-one; and 5-(2-hydroxy-4-methyl-2-(2-methyl-2-phenylpropyl)-4-phenylpentylamino)isobenzofuran-1(31H)-one.

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R² are incompatible under the reaction conditions of the process, protection/deprotection of these groups may be carried out, as required, using reagents and conditions readily selected by one of ordinary skill in the art (see, for example, T. W. Greene and P. G. M. Wuts, “Protective Groups in Organic Synthesis,” John Wiley & Sons, New York (1999)) and references cited therein. For example, a hydroxyl group can be protected as methyl ether and be deprotected at an appropriate stage with reagents, such as boron tribromide in dichloromethane. Typically, reaction progress may be monitored by high performance liquid chromatography (“HPLC”) or thin layer chromatography (“TLC”), if desired, and intermediates and products may be purified by chromatography on silica gel and/or by recrystallization.

In another aspect, a stereoisomer having Formula Ia, Ib, IIa, IIb, IIIa, or IIIb produced by a method of the present invention can be included in a pharmaceutical composition for treating, controlling, reducing, ameliorating, or preventing inflammation or infections and their inflammatory sequelae. In one embodiment, such a pharmaceutical composition is an ophthalmic pharmaceutical composition.

The compounds prepared by any method of the present invention disclosed herein, including compounds having Formula Ia, Ib, IIa, IIb, IIIa, IIIb, IIIc, or IIId, are preferably formulated prior to administration. Suitable pharmaceutical formulations are prepared by known procedures using additional well known and readily available ingredients. In making the compositions suitable for use in treating, controlling, reducing, ameliorating, or preventing inflammation or infections and their inflammatory sequelae, the active ingredient will usually be mixed with a carrier, or diluted by a carrier, or enclosed within a carrier which may be in the form of a capsule, sachet, paper or other container. When the carrier serves as a diluent, it may be a solid, semisolid, or liquid material, which acts as a vehicle, excipient, or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders for either oral or topical application.

Some examples of suitable carriers, excipient, and diluents include lactose, dextrose, sucrose sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions including a compound prepared by a method of the present invention of the invention may be formulated so as to provide quick, sustained or delayed release of the active ingredient after administration to the patient. The compositions are preferably formulated in a unit dosage form, each dosage containing from about 0.005 mg to about 5 g (alternatively, from about 0.01 mg to about 1 g, or from about 0.1 mg to about 0.5 g, or from about 1 mg to about 0.1 g) of the active ingredient. However, it will be understood that the therapeutic dosage administered will be determined by the physician in the light of the relevant circumstances including the severity of the condition to be treated, the choice of compound to be administered and the chosen route of administration. Therefore, the above dosage ranges are not intended to limit the scope of the invention in any way. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

In addition to the above formulations, most of which may be administered orally, the compounds used in a method of treatment disclosed herein also may be administered topically. Topical formulations include ointments, creams and gels.

Ointments generally are prepared using either (1) an oleaginous base, i.e., one consisting of fixed oils or hydrocarbons, such as white petrolatum or mineral oil, or (2) an absorbent base, i.e., one consisting of an anhydrous substance or substances which can absorb water, for example anhydrous lanolin. Customarily, following formation of the base, whether oleaginous or absorbent, the active ingredient (compound) is added to an amount affording the desired concentration.

Creams are oil/water emulsions. They consist of an oil phase (internal phase), comprising typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate, hydrophilic colloids, such as acacia colloidal clays, veegum, and the like. Upon formation of the emulsion, the active ingredient (compound) customarily is added in an amount to achieve the desired concentration.

Gels comprise a base selected from an oleaginous base, water, or an emulsion-suspension base. To the base is added a gelling agent which forms a matrix in the base, increasing its viscosity. Examples of gelling agents are hydroxypropyl cellulose, acrylic acid polymers, and the like. Customarily, the active ingredient (compound) is added to the formulation at the desired concentration at a point preceding addition of the gelling agent.

The amount of compound incorporated into a topical formulation is not critical; the concentration should be within a range sufficient to permit ready application of the formulation to the affected tissue area in an amount which will deliver the desired amount of compound to the desired treatment site.

The customary amount of a topical formulation to be applied to an affected tissue will depend upon an affected tissue size and concentration of the compound in the formulation. Generally, the formulation will be applied to the effected tissue in an amount affording from about 1 to about 500 μg of the compound per cm² of an affected tissue. Preferably, the applied amount of compound will range from about 30 to about 300 μg/cm², more preferably, from about 50 to about 200 μg/cm², and, most preferably, from about 60 to about 100 μg/cm².

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for selectively producing a stereoisomer of a substituted alcohol that has a Formula Ia or Ib, 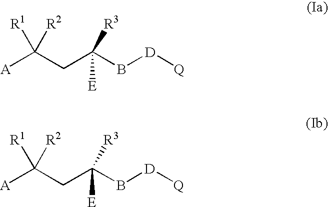
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; or R and R² together form an unsubstituted or substituted C₃-C₁₅ cycloalkyl group; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclic groups; B comprises a methylene or substituted methylene group, wherein one or two substituents on the methylene group are independently C₁-C₅ alkyl, hydroxy, halogen, amino, or oxo group; E is hydroxy; and D is —NH— or —NR′—, wherein R′ comprises an unsubstituted or substituted C₁-C₁₅ linear or branched alkyl group; the method comprising:
(a) converting a chiral epoxyester or epoxycarboxamide having Formula VIa or VIb to a chiral primary epoxyamine having Formula VIIa or VIIb under a halogen and in the presence of a base, or upon treatment with an alkali hypohalite; 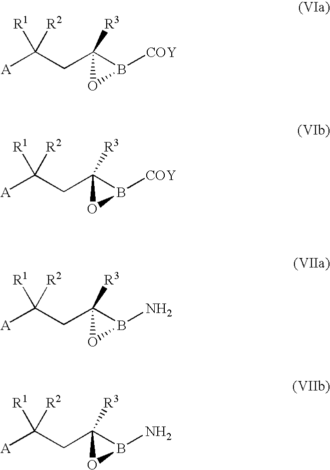
wherein Y is OR*, NH₂, or NHR*, and R* is a chiral auxiliary;
(b) reducing the chiral primary epoxyamine having Formula VIIa or VIIb to form a corresponding chiral primary amine having Formula IVa or IVb; and 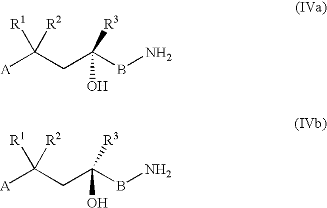
(c) reacting the chiral primary amine having Formula IVa or IVb with a compound having a formula of Q-X under a base catalysis condition or under transition metal catalysis, wherein X is a halogen, to selectively produce the compound having Formula Ia or Ib.

2. The method of claim 1, wherein R¹ and R² are independently selected from the group consisting of hydrogen, unsubstituted C₁-C₅ linear or branched alkyl groups, substituted C₁-C₅ linear or branched alkyl groups, unsubstituted C₃-C₅ cycloalkyl groups, and substituted C₃-C₅ cycloalkyl groups; or R¹ and R² together form an unsubstituted or substituted C₃-C₅ cycloalkyl group; R³ is selected from the group consisting of hydrogen, unsubstituted C₁-C₅ linear or branched alkyl groups, substituted C₁-C₅ linear or branched alkyl groups, unsubstituted C₃-C₅ cycloalkyl and heterocycloalkyl groups, and substituted C₃-C₅ cycloalkyl; B comprises a methylene or substituted methylene group, wherein one or two substituents on the methylene group are independently C₁-C₃ alkyl; E is hydroxy; and D is —NH— or —NR′—, wherein R′ comprises an unsubstituted or substituted C₁-C₅ linear or branched alkyl group.

3. A method for producing a single substituted alcohol steroisomer having Formula Ia or Ib, 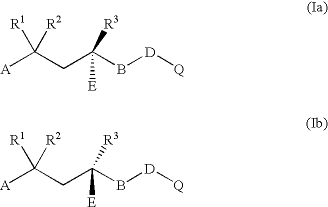
substantially free of the other stereoisomer, the method comprising:
(a) reacting a ketone having Formula VIII with a chiral ester of an α-haloacid under a basic condition to form a chiral epoxyester having Formula VIa or VIb 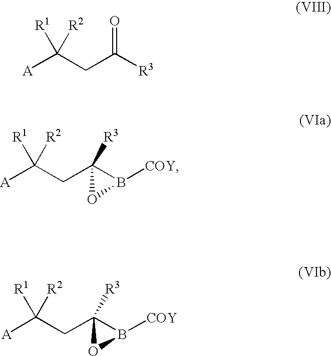
wherein Y is OR* and R* is a chiral auxiliary;
(b) reacting the chiral epoxyester having Formula VIa or VIb with ammonia or an amine to produce a chiral epoxycarboxamide having Formula IXa or IXb 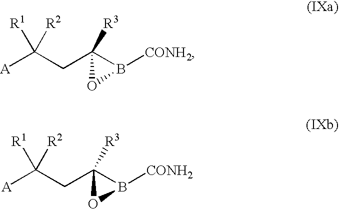
(c) converting the chiral epoxycarboxamide having Formula IXa or IXb to a chiral primary amine having Formula VIIa or VIIb under a halogen and in the presence of a base, or upon treatment with an alkali hypohalite 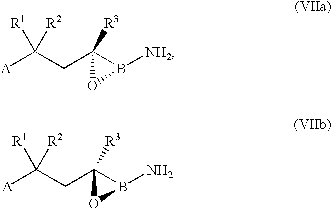
(d) carrying out the step of: (i) reducing the chiral primary epoxyamine having Formula VIIa or VIIb to form a chiral hydroxyamine having Formula IVa or IVb 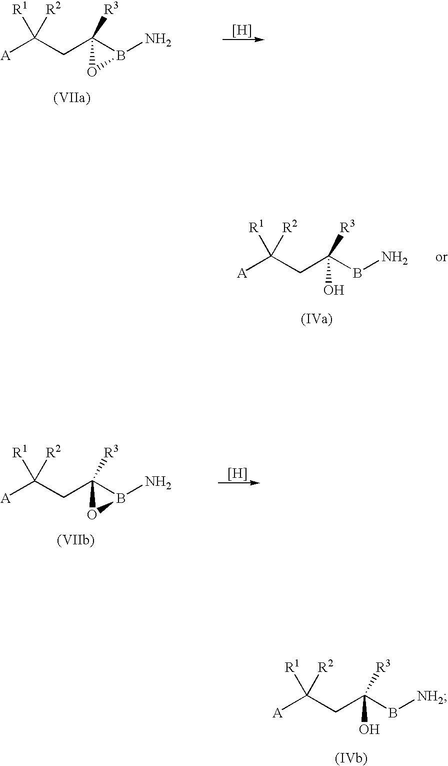
or (ii) converting the chiral primary epoxyamine having Formula VIIa or VIIb, under an acidic condition, to the chiral aldehyde or ketone having Formula Va or Vb; and 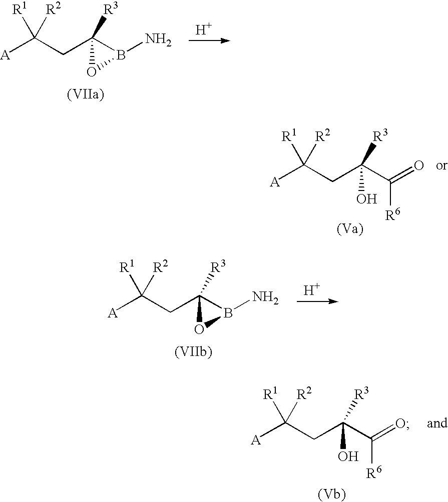
(e) reacting: (1) the chiral hydroxyamine having Formula IVa or IVb with a compound having a formula of Q-X under a base catalysis condition or under transition metal catalysis, when the step (d)(i) is carried out; or (2) the chiral aldehyde or ketone Va or Vb with a compound having a formula of Q-NH₂ or Q-NHR′, when the step (d)(ii) is carried out, to produce the single substituted alcohol stereoisomer having Formula Ia or Ib;
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; or R¹ and R² together form an unsubstituted or substituted C₃-C₁₅ cycloalkyl group; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclic groups; B comprises a methylene or substituted methylene group, wherein a substituent on the methylene group is C₁-C₅ alkyl, hydroxy, halogen, or amino; E is hydroxy; D is —NH— or —NR′—, wherein R′ comprises an unsubstituted or substituted C₁-C₁₅ linear or branched alkyl group; and X is a halogen or tosylate group.

4. The method of claim 3, wherein R¹ and R² are independently selected from the group consisting of hydrogen, unsubstituted C₁-C₅ linear or branched alkyl groups, substituted C₁-C₅ linear or branched alkyl groups, unsubstituted C₃-C₅ cycloalkyl groups, and substituted C₃-C₅ cycloalkyl groups; or R¹ and R² together form an unsubstituted or substituted C₃-C₅ cycloalkyl group; R³ is selected from the group consisting of hydrogen, unsubstituted C₁-C₅ linear or branched alkyl groups, substituted C₁-C₅ linear or branched alkyl groups, unsubstituted C₃-C₅ cycloalkyl and heterocycloalkyl groups, and substituted C₃-C₅ cycloalkyl; B comprises a methylene or substituted methylene group, wherein one or two substituents on the methylene group are independently C₁-C₃ alkyl; E is hydroxy; and D is —NH— or —NR′—, wherein R′ comprises an unsubstituted or substituted C₁-C₅ linear or branched alkyl group.

5. A method for producing a single substituted alcohol steroisomer having Formula Ia or Ib, 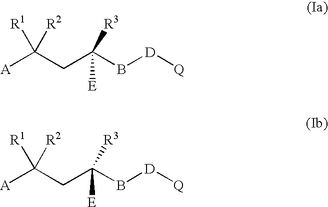
substantially free of the other stereoisomer, the method comprising:
(a) reacting a ketone having Formula VIII with an amide of an α-haloacid under a basic condition in the presence of a chiral catalyst to form a chiral epoxycarboxamide having Formula IXa or IXb 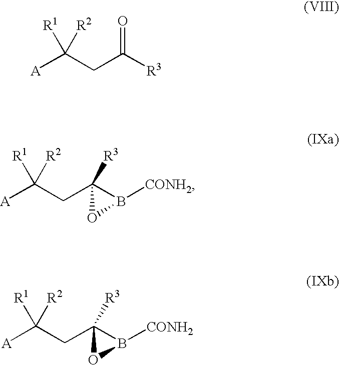
(b) converting the chiral epoxycarboxamide having Formula IXa or IXb to a chiral primary amine having Formula VIIa or VIIb under a halogen and in the presence of a base, or upon treatment with an alkali hypohalite 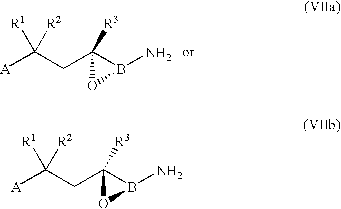
(c) carrying out the step of: (i) reducing the chiral primary epoxyamine having Formula VIIa or VIIb to form a chiral hydroxyamine having Formula IVa or IVb 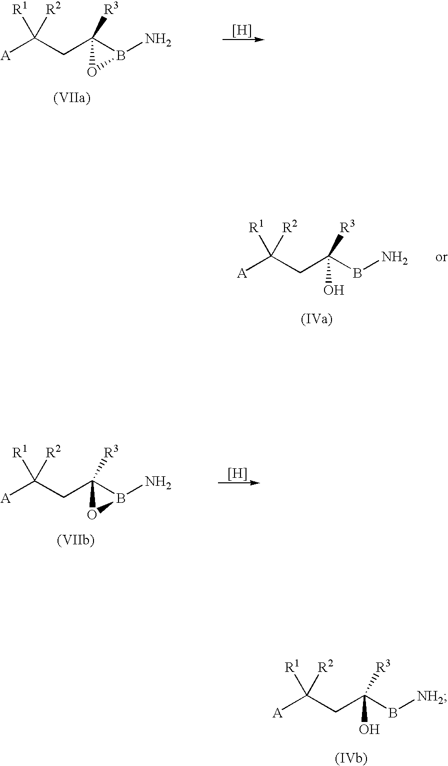
or (ii) converting the chiral primary epoxyamine having Formula VIIa or VIIb, under an acidic condition, to the chiral aldehyde or ketone having Formula Va or Vb; and 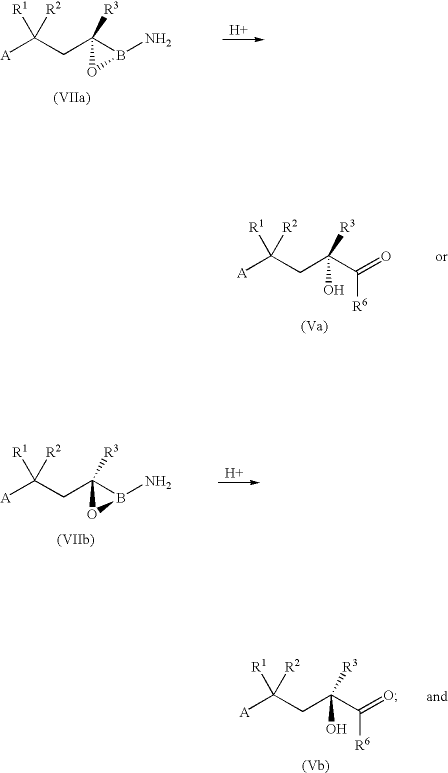
(d) reacting: (1) the chiral hydroxyamine having Formula IVa or IVb with a compound having a formula of Q-X under a base catalysis condition or under transition metal catalysis, when the step (c)(i) is carried out; or (2) the chiral aldehyde or ketone Va or Vb with a compound having a formula of Q-NH₂ or Q-NHR′, when the step (c)(ii) is carried out, to produce the single substituted alcohol stereoisomer having Formula Ia or Ib;
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; or R¹ and R² together form an unsubstituted or substituted C₃-C₁₅ cycloalkyl group; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclic groups; B comprises a methylene or substituted methylene group, wherein a substituent on the methylene group is C₁-C₅ alkyl, hydroxy, halogen, or amino; E is hydroxy; D is —NH— or —NR′—, wherein R′ comprises an unsubstituted or substituted C₁-C₁₅ linear or branched alkyl group; and X is a halogen or tosylate group.

6. The method of claim 5, wherein R¹ and R² are independently selected from the group consisting of hydrogen, unsubstituted C₁-C₅ linear or branched alkyl groups, substituted C₁-C₅ linear or branched alkyl groups, unsubstituted C₃-C₅ cycloalkyl groups, and substituted C₃-C₅ cycloalkyl groups; or R¹ and R² together form an unsubstituted or substituted C₃-C₅ cycloalkyl group; R³ is selected from the group consisting of hydrogen, unsubstituted C₁-C₅ linear or branched alkyl groups, substituted C₁-C₅ linear or branched alkyl groups, unsubstituted C₃-C₅ cycloalkyl and heterocycloalkyl groups, and substituted C₃-C₅ cycloalkyl; B comprises a methylene or substituted methylene group, wherein one or two substituents on the methylene group are independently C₁-C₃ alkyl; E is hydroxy; and D is —NH— or —NR′—, wherein R′ comprises an unsubstituted or substituted C₁-C₅ linear or branched alkyl group.

7. The method of claim 2, wherein A and Q are independently selected from the group consisting of unsubstituted and substituted aryl and heteroaryl groups, and unsubstituted and substituted heterocyclic groups.

8. The method of claim 2, wherein A is an unsubstituted aryl, substituted aryl, unsubstituted heteroaryl, or substituted heteroaryl group, and Q is an unsubstituted or substituted azaindolyl group.

9. The method of claim 2, wherein A is an unsubstituted aryl, substituted aryl, unsubstituted heteroaryl, or substituted heteroaryl group, and Q is a methylated benzoxazinone group.

10. The method of claim 2, wherein A is an unsubstituted aryl, substituted aryl, unsubstituted heteroaryl, or substituted heteroaryl group, and Q comprises a quinoline, isoquinoline, pyrrolidine, morpholine, thiomorpholine, piperazine, piperidine, 1H-pyridin-4-one, 1H-pyridin-2-one, 1H-pyridin-4-ylideneamine, 1H-quinolin-4-ylideneamine, pyran, tetrahydropyran, 1,4-diazepane, 2,5-diazabicyclo[2.2.1]heptane, 2,3,4,5-tetrahydrobenzo[b][1,4]diazepine, dihydroquinoline, tetrahydroquinoline, 5,6,7,8-tetrahydro-1H-quinolin-4-one, tetrahydroisoquinoline, decahydroisoquinoline, 2,3-dihydro-1H-isoindole, 2,3-dihydro-1H-indole, chroman, 1,2,3,4-tetrahydroquinoxaline, 1,2-dihydroindazol-3-one, 3,4-dihydro-2H-benzo[1,4]oxazine, 4H-benzo[1,4]thiazine, 3,4-dihydro-2H-benzo[1,4]thiazine, 1,2-dihydrobenzo[d][1,3]oxazin-4-one, 3,4-dihydrobenzo[1,4]oxazin-4-one, 3H-quinazolin-4-one, 3,4-dihydro-1H-quinoxalin-2-one, 1H-quinnolin-4-one, 1H-quinazolin-4-one, 1H-[1,5]naphthyridin-4-one, 5,6,7,8-tetrahydro-1H-[1,-5]naphthyridin-4-one, 2,3-dihydro-1H-[1,5]naphthyridin-4-one, 1,2-dihydropyrido[3,2-d][1,3]oxazin-4-one, pyrrolo[3,4-c]pyridine-1,3-dione, 1,2-dihydropyrrolo[3,4-c]pyridin-3-one, or tetrahydro[b][1,4]diazepinone group, each unsubstituted or independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, oxo, cyano, trifluoromethyl, trifluoromethoxy, trifluoromethylthio, nitro, amino wherein the nitrogen atom is unsubstituted or independently mono- or di-substituted by C₁-C₅ alkyl, ureido wherein either nitrogen atom is unsubstituted or independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from C₁-C₃ alkyl, C₁-C₃ alkoxy, C₁-C₃ alkoxycarbonyl, acyl, aryl, benzyl, heteroaryl, heterocyclyl, halogen, hydroxy, oxo, cyano, amino wherein the nitrogen atom is optionally independently mono- or di-substituted by C₁-C₅ alkyl, or ureido wherein either nitrogen atom is optionally independently substituted with C₁-C₅ alkyl.

11. The method of claim 2, wherein A is an unsubstituted aryl, substituted aryl, unsubstituted heteroaryl, or substituted heteroaryl group, and Q comprises an unsubstituted or substituted phenyl group having the formula 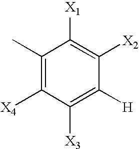
wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein t h e nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

12. The method of claim 2, wherein A is an unsubstituted aryl, substituted aryl, unsubstituted heteroaryl, or substituted heteroaryl group, and Q comprises an unsubstituted or substituted indolyl group with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from the group consisting of C₁-C₃ alkyl, C₁-C₃ alkoxy, halogen, hydroxy, oxo, cyano, amino, and trifluoromethyl.

13. The method of claim 2, wherein A is an unsubstituted aryl, substituted aryl, unsubstituted heteroaryl, or substituted heteroaryl group; and Q comprises the group

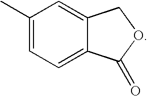

14. The method of claim 4, wherein A and Q are independently selected from the group consisting of unsubstituted and substituted aryl and heteroaryl groups, and unsubstituted and substituted heterocyclic groups.

15. The method of claim 4, wherein A is an unsubstituted aryl, substituted aryl, unsubstituted heteroaryl, or substituted heteroaryl group, and Q is an unsubstituted or substituted azaindolyl group.

16. The method of claim 4, wherein A is an unsubstituted aryl, substituted aryl, unsubstituted heteroaryl, or substituted heteroaryl group, and Q is a methylated benzoxazinone group.

17. The method of claim 4, wherein A is an unsubstituted aryl, substituted aryl, unsubstituted heteroaryl, or substituted heteroaryl group, and Q comprises a quinoline, isoquinoline, pyrrolidine, morpholine, thiomorpholine, piperazine, piperidine, 1H-pyridin-4-one, 1H-pyridin-2-one, 1H-pyridin-4-ylideneamine, 1H-quinolin-4-ylideneamine, pyran, tetrahydropyran, 1,4-diazepane, 2,5-diazabicyclo[2.2.1]heptane, 2,3,4,5-tetrahydrobenzo[b][1,4]diazepine, dihydroquinoline, tetrahydroquinoline, 5,6,7,8-tetrahydro-1H-quinolin-4-one, tetrahydroisoquinoline, decahydroisoquinoline, 2,3-dihydro-1H-isoindole, 2,3-dihydro-1H-indole, chroman, 1,2,3,4-tetrahydroquinoxaline, 1,2-dihydroindazol-3-one, 3,4-dihydro-2H-benzo[1,4]oxazine, 4H-benzo[1,4]thiazine, 3,4-dihydro-2H-benzo[1,4]thiazine, 1,2-dihydrobenzo[d][1,3]oxazin-4-one, 3,4-dihydrobenzo[1,4]oxazin-4-one, 3H-quinazolin-4-one, 3,4-dihydro-1H-quinoxalin-2-one, 1H-quinnolin-4-one, 1H-quinazolin-4-one, 1H-[1,5]naphthyridin-4-one, 5,6,7,8-tetrahydro-1H-[1,-5]naphthyridin-4-one, 2,3-dihydro-1H-[1,5]naphthyridin-4-one, 1,2-dihydropyrido[3,2-d][1,3]oxazin-4-one, pyrrolo[3,4-c]pyridine-1,3-dione, 1,2-dihydropyrrolo[3,4-c]pyridin-3-one, or tetrahydro[b][1,4]diazepinone group, each unsubstituted or independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, oxo, cyano, trifluoromethyl, trifluoromethoxy, trifluoromethylthio, nitro, amino wherein the nitrogen atom is unsubstituted or independently mono- or di-substituted by C₁-C₅ alkyl, ureido wherein either nitrogen atom is unsubstituted or independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from C₁-C₃ alkyl, C₁-C₃ alkoxy, C₁-C₃ alkoxycarbonyl, acyl, aryl, benzyl, heteroaryl, heterocyclyl, halogen, hydroxy, oxo, cyano, amino wherein the nitrogen atom is optionally independently mono- or di-substituted by C₁-C₅ alkyl, or ureido wherein either nitrogen atom is optionally independently substituted with C₁-C₅ alkyl.

18. The method of claim 4, wherein A is an unsubstituted aryl, substituted aryl, unsubstituted heteroaryl, or substituted heteroaryl group, and Q comprises an unsubstituted or substituted phenyl group having the formula 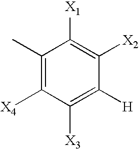
wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein the nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

19. The method of claim 4, wherein A is an unsubstituted aryl, substituted aryl, unsubstituted heteroaryl, or substituted heteroaryl group, and Q comprises an unsubstituted or substituted indolyl group with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from the group consisting of C₁-C₃ alkyl, C₁-C₃ alkoxy, halogen, hydroxy, oxo, cyano, amino, and trifluoromethyl.

20. The method of claim 4, wherein A is an unsubstituted aryl, substituted aryl, unsubstituted heteroaryl, or substituted heteroaryl group; and Q comprises the group

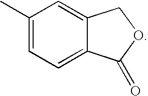

21. The method of claim 6, wherein A and Q are independently selected from the group consisting of unsubstituted and substituted aryl and heteroaryl groups, and unsubstituted and substituted heterocyclic groups.

22. The method of claim 6, wherein A is an unsubstituted aryl, substituted aryl, unsubstituted heteroaryl, or substituted heteroaryl group, and Q is an unsubstituted or substituted azaindolyl group.

23. The method of claim 6, wherein A is an unsubstituted aryl, substituted aryl, unsubstituted heteroaryl, or substituted heteroaryl group, and Q is a methylated benzoxazinone group.

24. The method of claim 6, wherein A is an unsubstituted aryl, substituted aryl, unsubstituted heteroaryl, or substituted heteroaryl group, and Q comprises a quinoline, isoquinoline, pyrrolidine, morpholine, thiomorpholine, piperazine, piperidine, 1H-pyridin-4-one, 1H-pyridin-2-one, 1H-pyridin-4-ylideneamine, 1H-quinolin-4-ylideneamine, pyran, tetrahydropyran, 1,4-diazepane, 2,5-diazabicyclo[2.2.1]heptane, 2,3,4,5-tetrahydrobenzo[b][1,4]diazepine, dihydroquinoline, tetrahydroquinoline, 5,6,7,8-tetrahydro-1H-quinolin-4-one, tetrahydroisoquinoline, decahydroisoquinoline, 2,3-dihydro-1H-isoindole, 2,3-dihydro-1H-indole, chroman, 1,2,3,4-tetrahydroquinoxaline, 1,2-dihydroindazol-3-one, 3,4-dihydro-2H-benzo[1,4]oxazine, 4H-benzo[1,4]thiazine, 3,4-dihydro-2H-benzo[1,4]thiazine, 1,2-dihydrobenzo[d][1,3]oxazin-4-one, 3,4-dihydrobenzo[1,4]oxazin-4-one, 3H-quinazolin-4-one, 3,4-dihydro-1H-quinoxalin-2-one, 1H-quinnolin-4-one, 1H-quinazolin-4-one, 1H-[1,5]naphthyridin-4-one, 5,6,7,8-tetrahydro-1H-[1,-5]naphthyridin-4-one, 2,3-dihydro-1H-[1,5]naphthyridin-4-one, 1,2-dihydropyrido[3,2-d][1,3]oxazin-4-one, pyrrolo[3,4-c]pyridine-1,3-dione, 1,2-dihydropyrrolo[3,4-c]pyridin-3-one, or tetrahydro[b][1,4]diazepinone group, each unsubstituted or independently substituted with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, oxo, cyano, trifluoromethyl, trifluoromethoxy, trifluoromethylthio, nitro, amino wherein the nitrogen atom is unsubstituted or independently mono- or di-substituted by C₁-C₅ alkyl, ureido wherein either nitrogen atom is unsubstituted or independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from C₁-C₃ alkyl, C₁-C₃ alkoxy, C₁-C₃ alkoxycarbonyl, acyl, aryl, benzyl, heteroaryl, heterocyclyl, halogen, hydroxy, oxo, cyano, amino wherein the nitrogen atom is optionally independently mono- or di-substituted by C₁-C₅ alkyl, or ureido wherein either nitrogen atom is optionally independently substituted with C₁-C₅ alkyl.

25. The method of claim 6, wherein A is an unsubstituted aryl, substituted aryl, unsubstituted heteroaryl, or substituted heteroaryl group, and Q comprises an unsubstituted or substituted phenyl group having the formula 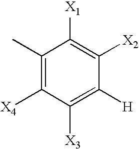
wherein X₁, X₂, X₃ and X₄ are each independently selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl, and amino wherein the nitrogen atom may be independently mono- or di-substituted by C₁-C₅ alkyl, and wherein said aryl group is optionally substituted by one or more hydroxy or C₁-C₅ alkoxy groups, and wherein either nitrogen atom of the urea group may be independently substituted by C₁-C₅ alkyl; or Q is an aromatic 5- to 7-membered monocyclic ring having from one to four heteroatoms in the ring independently selected from nitrogen, oxygen, and sulfur, optionally independently substituted with one to three substituent groups selected from the group consisting of hydrogen, halogen, hydroxy, trifluoromethyl, trifluoromethoxy, C₁-C₅ alkyl, C₂-C₅ alkenyl, C₂-C₅ alkynyl, C₁-C₅ alkoxy, C₁-C₅ alkylthio wherein the sulfur atom is optionally oxidized to a sulfoxide or sulfone, C₁-C₅ alkanoyl, C₁-C₅ alkoxycarbonyl, C₁-C₅ acyloxy, C₁-C₅ alkanoylamino, C₁-C₅ carbamoyloxy, urea, aryl optionally substituted by one or more hydroxy or C₁-C₅ alkoxy groups, and amino wherein the nitrogen atom may be independently mono- or di-substituted by C₁-C₅ alkyl, and wherein either nitrogen atom of the urea group may be independently substituted by C₁-C₅ alkyl.

26. The method of claim 6, wherein A is an unsubstituted aryl, substituted aryl, unsubstituted heteroaryl, or substituted heteroaryl group, and Q comprises an unsubstituted or substituted indolyl group with one to three substituent groups, wherein each substituent group of Q is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, amino wherein the nitrogen atom is optionally independently mono- or di-substituted by C₁-C₅ alkyl, ureido wherein either nitrogen atom is optionally independently substituted with C₁-C₅ alkyl, or C₁-C₅ alkylthio wherein the sulfur atom is optionally oxidized to a sulfoxide or sulfone, wherein each substituent group of Q is unsubstituted or independently substituted with one to three substituent groups selected from the group consisting of C₁-C₃ alkyl, C₁-C₃ alkoxy, halogen, hydroxy, oxo, cyano, amino, and trifluoromethyl.

27. The method of claim 6, wherein A is an unsubstituted aryl, substituted aryl, unsubstituted heteroaryl, or substituted heteroaryl group; and Q comprises the group

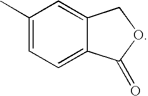

28. A method for producing a single substituted alcohol steroisomer having Formula IIa or IIb, 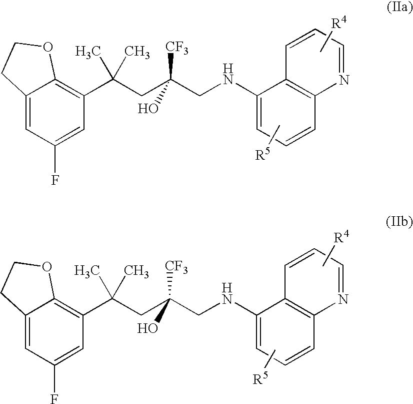
substantially free of the other stereoisomer, the method comprising:
(a) reacting a ketone having Formula X with a chiral ester of an α-haloacid under a basic condition to form a chiral epoxyester having Formula XIa or XIb 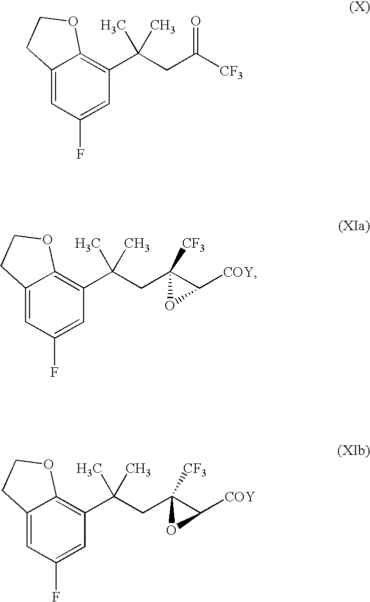
wherein Y is OR* and R* is a chiral auxiliary;
(b) reacting the chiral epoxyester having Formula XIa or XIb with ammonia or an amine to produce a chiral epoxycarboxamide having Formula XIIa or XIIb 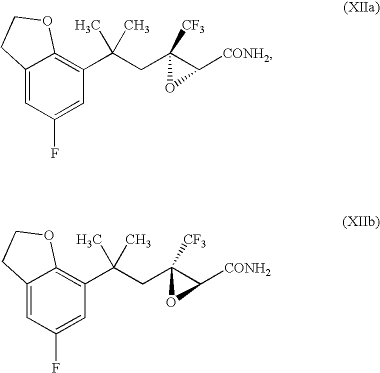
(c) converting the chiral epoxycarboxamide having Formula XIIa or XIIb to a chiral primary epoxyamine having Formula XIIIa or XIIIb under a halogen and in the presence of a base, or upon treatment with an alkali hypohalite; 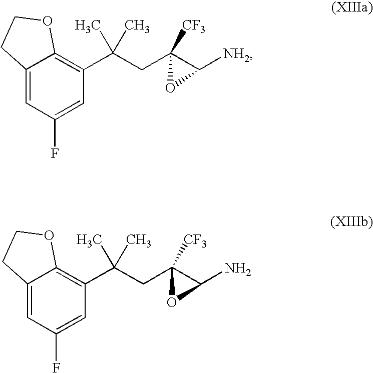
(d) carrying out the step of (i) reducing the chiral primary epoxyamine having Formula XIIIa or XIIIb to form a chiral hydroxyamine having Formula XIVa or XIVb; 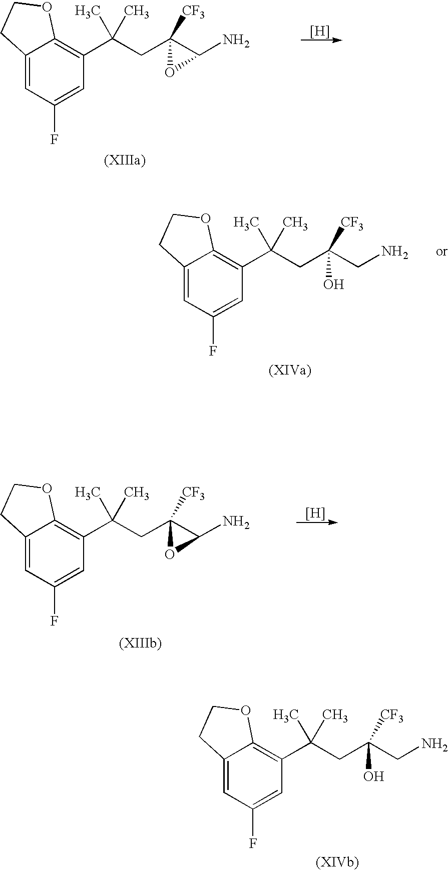
or (ii) converting the chiral primary epoxyamine having Formula XIIIa or XIIIb, under an acidic condition, to a chiral aldehyde having Formula XVa or XVb; 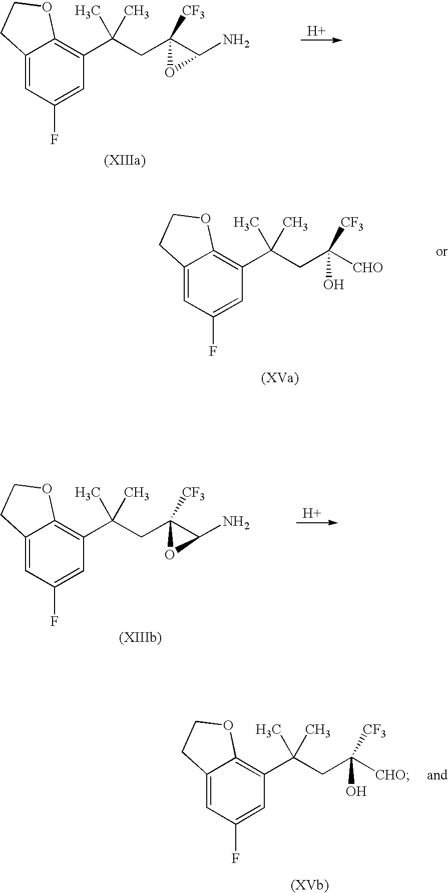
(e) reacting: (1) the chiral hydroxyamine having Formula XIVa or XIVb with a compound having a formula of Q-X under a base catalysis condition or under transition metal catalysis, when the step (d)(i) is carried out; or (2) the chiral aldehyde XVa or XVb with a compound having a formula of Q-NH₂, when the step (d)(ii) is carried out, to produce the single substituted alcohol stereoisomer having Formula IIa or IIb;
wherein Q is the quinolin-5-yl group which is unsubstituted or substituted at one or more positions 2, 3, 4, 6, 7, or 8; X is a halogen or tosylate group attached to the quinolinyl group at the 5 position; and R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

29. A method for producing a single substituted alcohol steroisomer having Formula IIc or IId, 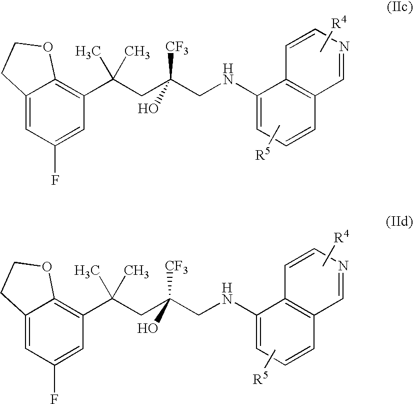
substantially free of the other stereoisomer, the method comprising:
(a) reacting a ketone having Formula X with a chiral ester of an α-haloacid under a basic condition to form a chiral epoxyester having Formula XIa or XIb 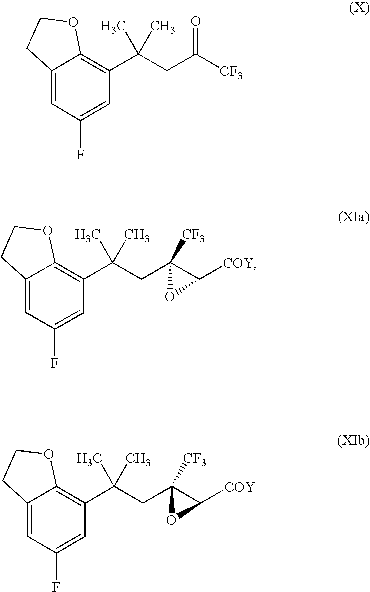
wherein Y is OR* and R* is a chiral auxiliary;
(b) reacting the chiral epoxyester having Formula XIa or XIb with ammonia or an amine to produce a chiral epoxycarboxamide having Formula XIIa or XIIb 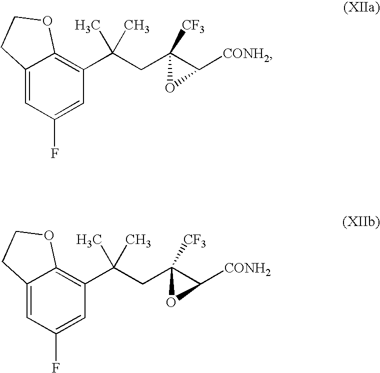
(c) converting the chiral epoxycarboxamide having Formula XIIa or XIIb to a chiral primary epoxyamine having Formula XIIIa or XIIIb under a halogen and in the presence of a base, or upon treatment with an alkali hypohalite; 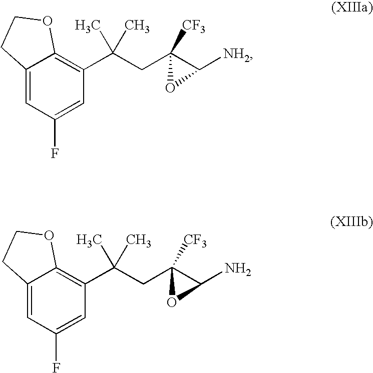
(d) carrying out the step of (i) reducing the chiral primary epoxyamine having Formula XIIIa or XIIIb to form a chiral hydroxyamine having Formula XIVa or XIVb; 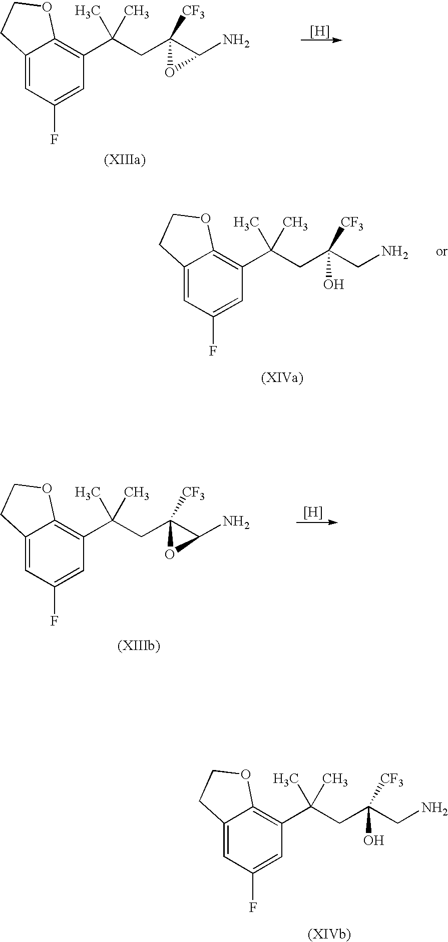
or (ii) converting the chiral primary epoxyamine having Formula XIIIa or XIIIb, under an acidic condition, to a chiral aldehyde having Formula XVa or XVb; 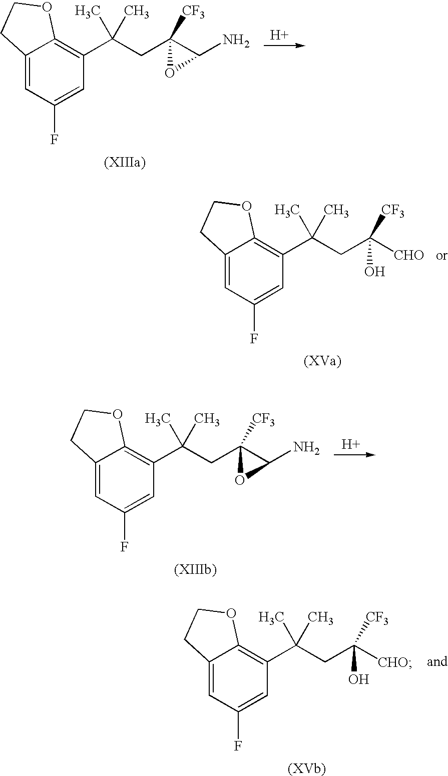
(e) reacting: (1) the chiral hydroxyamine having Formula XIVa or XIVb with a compound having a formula of Q-X under a base catalysis condition or under transition metal catalysis, when the step (d)(i) is carried out; or (2) the chiral aldehyde XVa or XVb with a compound having a formula of Q-NH₂, when the step (d)(ii) is carried out, to produce the single substituted alcohol stereoisomer having Formula IIa or IIb;
wherein Q is the isoquinolin-4-yl group which is unsubstituted or substituted at one or more positions 2, 3, 5, 6, 7, or 8; X is a halogen or tosylate group attached to the quinolinyl group at the 4 position; and R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

30. A method for producing a single substituted alcohol steroisomer having Formula IIa or IIb, 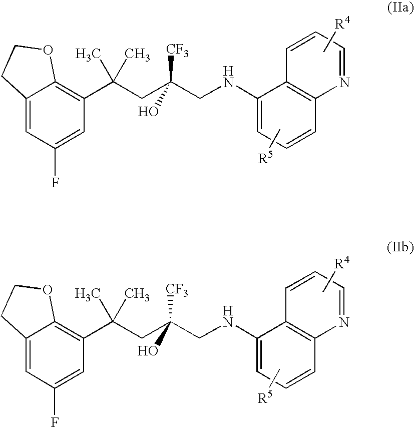
substantially free of the other stereoisomer, the method comprising:
(a) reacting a ketone having Formula X with a with an amide of an α-haloacid under a basic condition in the presence of a chiral catalyst to form a chiral epoxycarboxamide having Formula having Formula XIa or XIb; 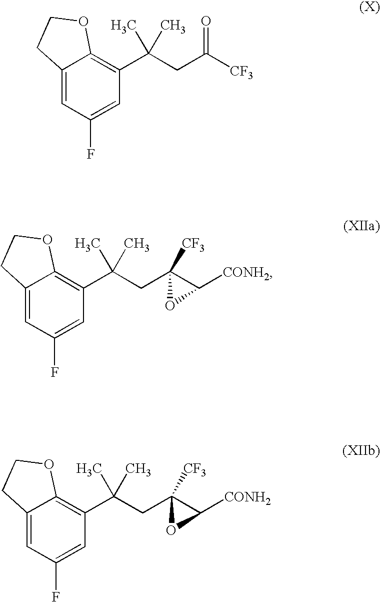
(b) converting the chiral epoxycarboxamide having Formula XIIa or XIIb to a chiral primary amine having Formula XIIIa or XIIIb under a halogen and in the presence of a base, or upon treatment with an alkali hypohalite; 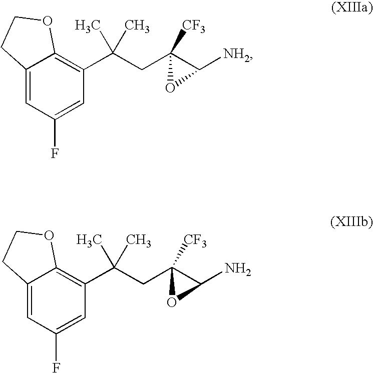
(c) carrying out the step of (i) reducing the chiral primary epoxyamine having Formula XIIIa or XIIIb to form a chiral hydroxyamine having Formula XIVa or XIVb; 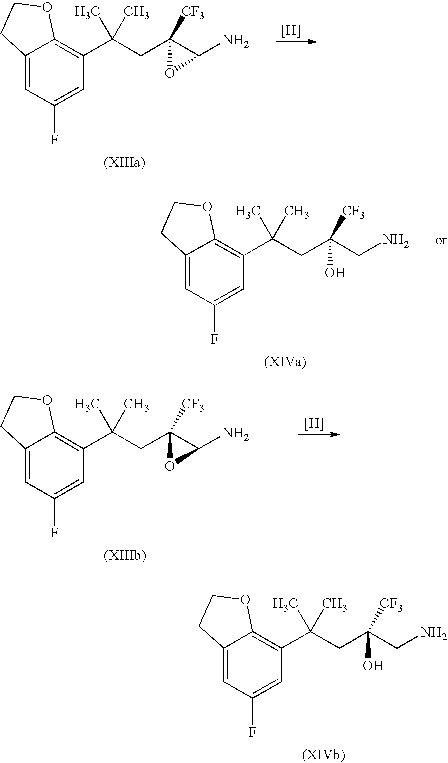
or (ii) converting the chiral primary epoxyamine having Formula XIIIa or XIIIb, under an acidic condition, to a chiral aldehyde having Formula XVa or XVb; 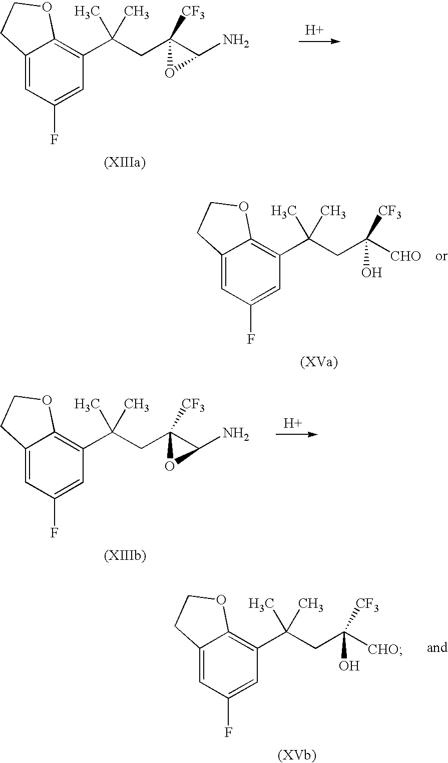
(d) reacting: (1) the chiral hydroxyamine having Formula XIVa or XIVb with a compound having a formula of Q-X under a base catalysis condition or under transition metal catalysis, when the step (c)(i) is carried out; or (2) the chiral aldehyde XVa or XVb with a compound having a formula of Q-NH₂, when the step (c)(ii) is carried out, to produce the single substituted alcohol stereoisomer having Formula IIa or IIb;
wherein Q is the quinolin-5-yl group which is unsubstituted or substituted at one or more positions 2, 3, 4, 6, 7, or 8; X is a halogen or tosylate group attached to the quinolinyl group at the 5 position; and R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

31. A method for producing a single substituted alcohol steroisomer having Formula IIc or IId, 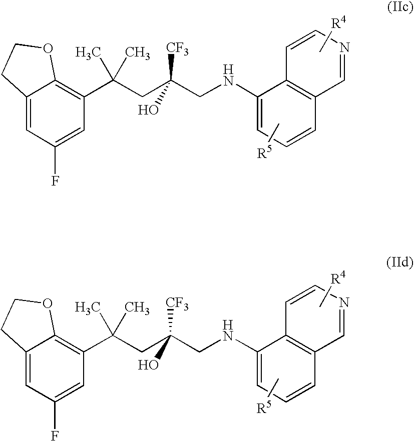
substantially free of the other stereoisomer, the method comprising:
(a) reacting a ketone having Formula X with a with an amide of an α-haloacid under a basic condition in the presence of a chiral catalyst to form a chiral epoxycarboxamide having Formula having Formula XIa or XIb; 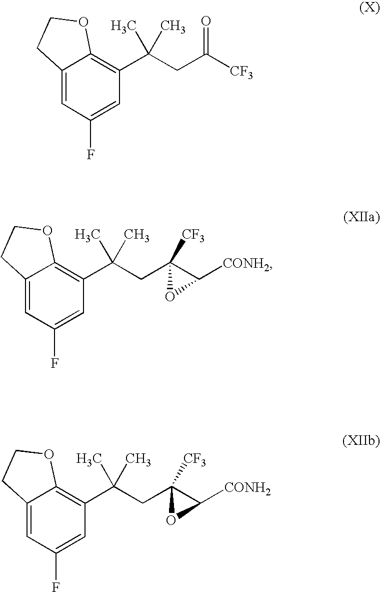
(b) converting the chiral epoxycarboxamide having Formula XIIa or XIIb to a chiral primary amine having Formula XIIIa or XIIIb under a halogen and in the presence of a base, or upon treatment with an alkali hypohalite; 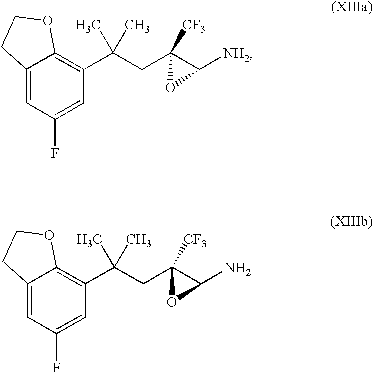
(c) carrying out the step of (i) reducing the chiral primary epoxyamine having Formula XIIIa or XIIIb to form a chiral hydroxyamine having Formula XIVa or XIVb; 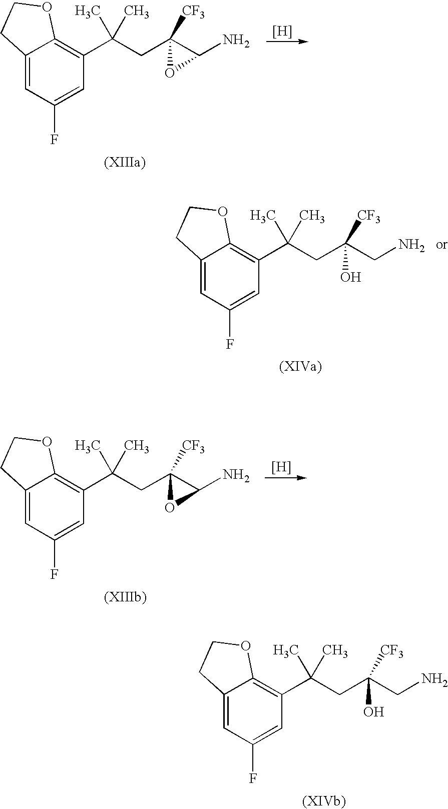
or (ii) converting the chiral primary epoxyamine having Formula XIIIa or XIIIb, under an acidic condition, to a chiral aldehyde having Formula XVa or XVb; 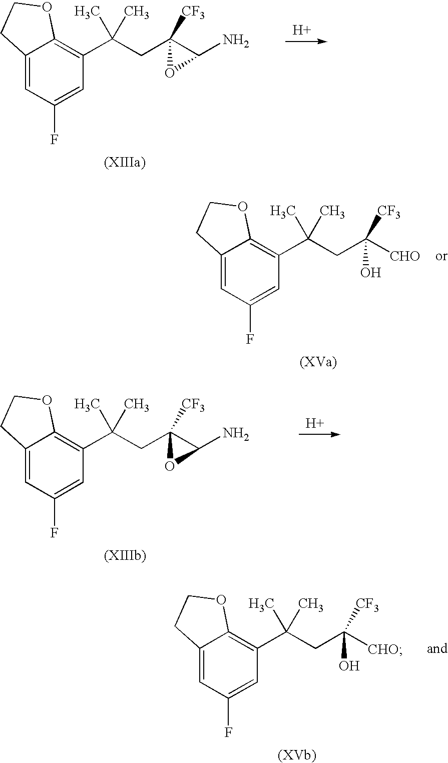
(e) reacting: (1) the chiral hydroxyamine having Formula XIVa or XIVb with a compound having a formula of Q-X under a base catalysis condition or under transition metal catalysis, when the step (c)(i) is carried out; or (2) the chiral aldehyde XVa or XVb with a compound having a formula of Q-NH₂, when the step (c)(ii) is carried out, to produce the single substituted alcohol stereoisomer having Formula IIa or IIb;
wherein Q is the isoquinolin-4-yl group which is unsubstituted or substituted at one or more positions 2, 3, 5, 6, 7, or 8; X is a halogen or tosylate group attached to the quinolinyl group at the 4 position; and R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

32. A method for producing a single substituted alcohol steroisomer having Formula IIIa or IIIb, 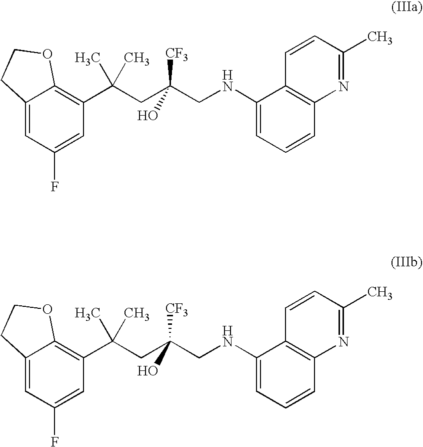
substantially free of the other stereoisomer, the method comprising:
(a) reacting a ketone having Formula X with a chiral ester of an α-haloacid under a basic condition to form a chiral epoxyester having Formula XIa or XIb; 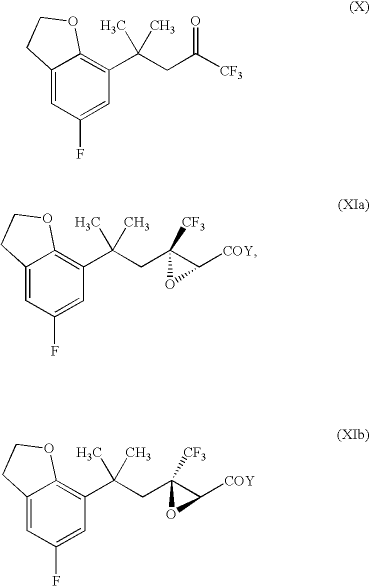
wherein Y is OR* and R* is a chiral auxiliary;
(b) reacting the chiral epoxyester having Formula XIa or XIb with ammonia or an amine to produce a chiral epoxycarboxamide having Formula XIIIa or XIIIb; 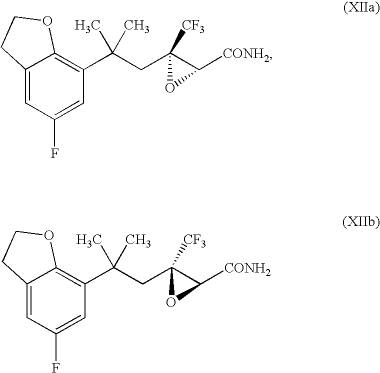
(c) converting the chiral epoxycarboxamide having Formula XIIa or XIIb to a chiral primary amine having Formula XIIIa or XIIIb under a halogen and in the presence of a base, or upon treatment with an alkali hypohalite; 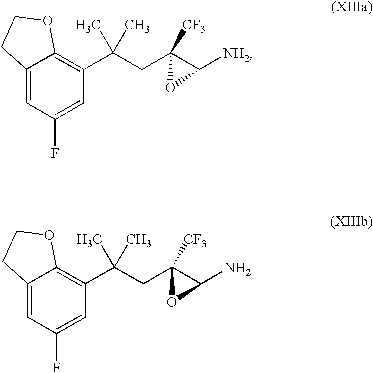
(d) carrying out the step of (i) reducing the chiral primary epoxyamine having Formula XIIIa or XIIIb to form a chiral hydroxyamine having Formula XIVa or XIVb; 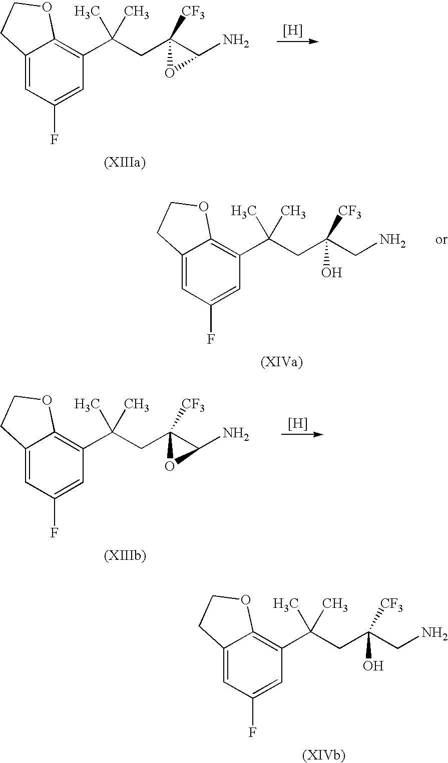
or (ii) converting the chiral primary epoxyamine having Formula XIIIa or XIIIb, under an acidic condition, to a chiral aldehyde having Formula XVa or XVb; 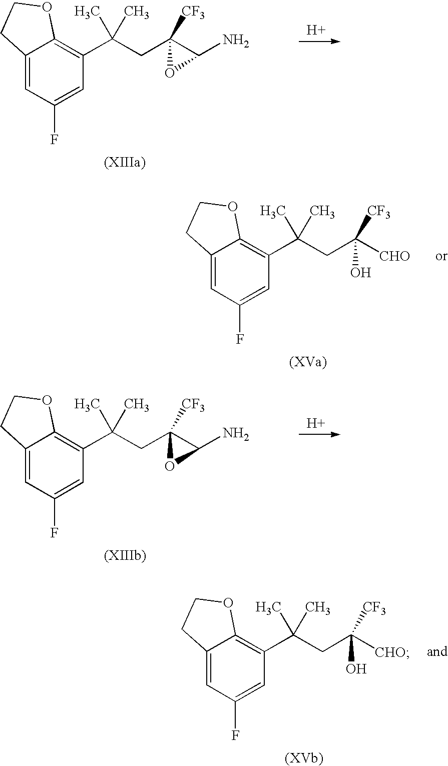
(e) reacting: (1) the chiral hydroxyamine having Formula XIVa or XIVb with a compound having a formula of 5-halo-2-methyl-quinoline under a base catalysis condition or under transition metal catalysis, when the step (d)(i) is carried out; or (2) the chiral aldehyde XVa or XVb with a compound having a formula of 5-amion-2-methyl-quinoline, when the step (d)(ii) is carried out, to produce the single substituted alcohol stereoisomer having Formula IIIa or IIIb; wherein the halo substituent at the 5 position on the substituted quinoline is selected from the group consisting bromine, chlorine, fluorine, and iodine.

33. A method for producing a single substituted alcohol steroisomer having Formula IIIc or IIId, 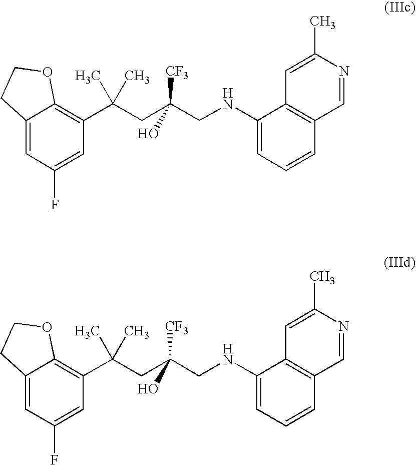
substantially free of the other stereoisomer, the method comprising:
(a) reacting a ketone having Formula X with a chiral ester of an α-haloacid under a basic condition to form a chiral epoxyester having Formula XIa or XIb; 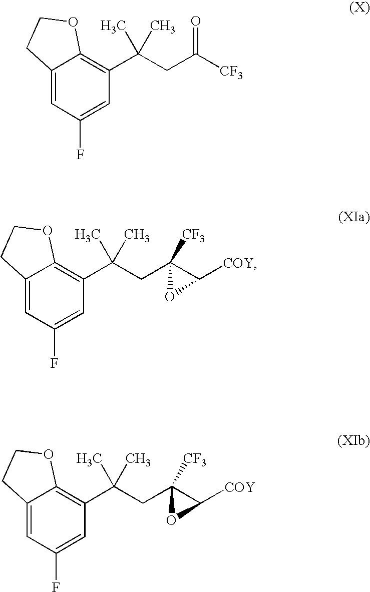
wherein Y is OR* and R* is a chiral auxiliary;
(b) reacting the chiral epoxyester having Formula XIa or XIb with ammonia or an amine to produce a chiral epoxycarboxamide having Formula XIIa or XIIb; 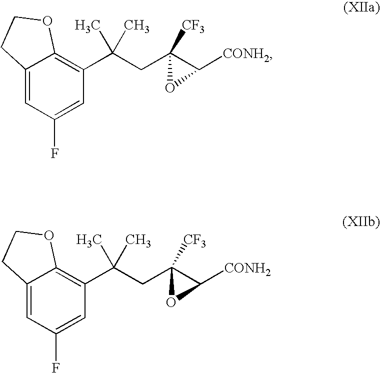
(c) converting the chiral epoxycarboxamide having Formula XIIa or XIIb to a chiral primary amine having Formula XIIIa or XIIIb under a halogen and in the presence of a base, or upon treatment with an alkali hypohalite; 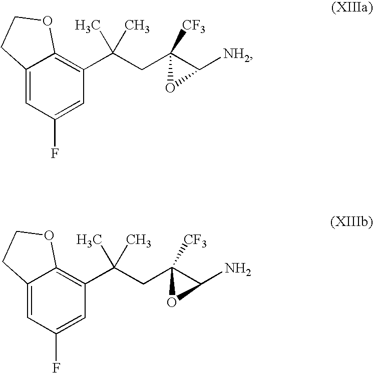
(d) carrying out the step of (i) reducing the chiral primary epoxyamine having Formula XIIIa or XIIIb to form a chiral hydroxyamine having Formula XIVa or XIVb; 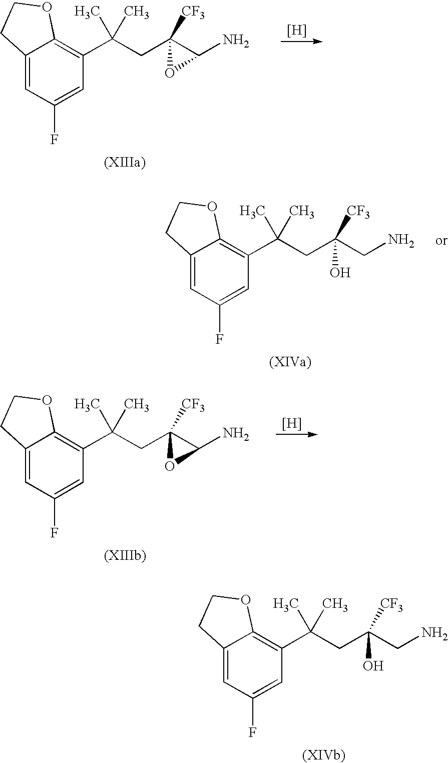
or (ii) converting the chiral primary epoxyamine having Formula XIIIa or XIIIb, under an acidic condition, to a chiral aldehyde having Formula XVa or XVb; 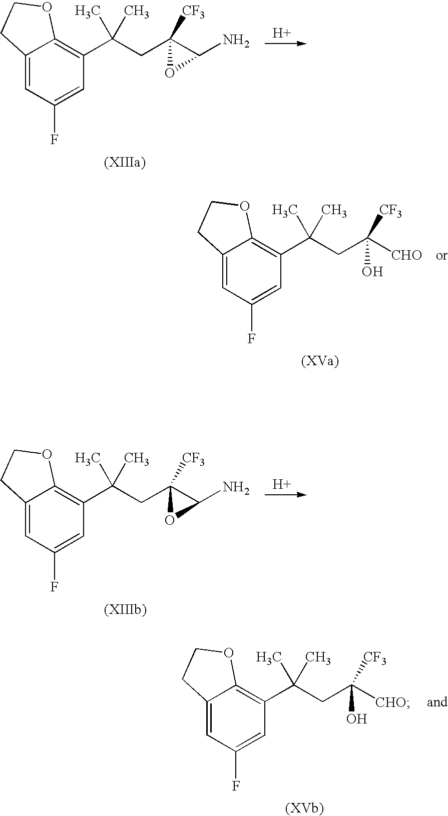
(e) reacting: (1) the chiral hydroxyamine having Formula XIVa or XIVb with a compound having a formula of 4-halo-2-methyl-isoquinoline under a base catalysis condition or under transition metal catalysis, when the step (d)(i) is carried out; or (2) the chiral aldehyde XVa or XVb with a compound having a formula of 4-amino-2-methyl-isoquinoline, when the step (d)(ii) is carried out, to produce the single substituted alcohol stereoisomer having Formula IIIa or IIIb; wherein X is a halogen substituent at the 4 position on the substituted isoquinoline and is selected from the group consisting bromine, chlorine, fluorine, and iodine.

34. A method for producing a single substituted alcohol steroisomer having Formula IIIa or IIIb, 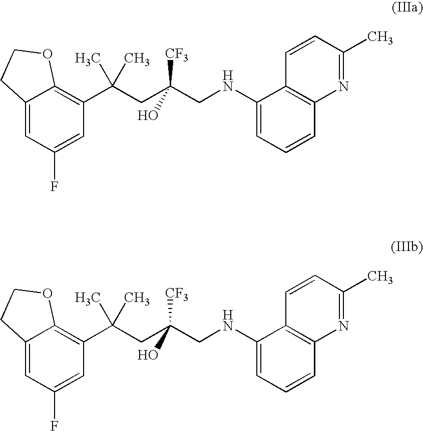
substantially free of the other stereoisomer, the method comprising:
(a) reacting a ketone having Formula X with a with an amide of an α-haloacid under a basic condition in the presence of a chiral catalyst to form a chiral epoxycarboxamide having Formula having Formula XIa or XIb; 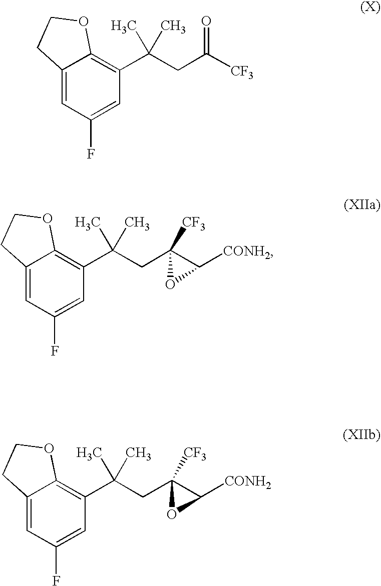
(b) converting the chiral epoxycarboxamide having Formula XIIa or XIIb to a chiral primary amine having Formula XIIIa or XIIIb under a halogen and in the presence of a base, or upon treatment with an alkali hypohalite; 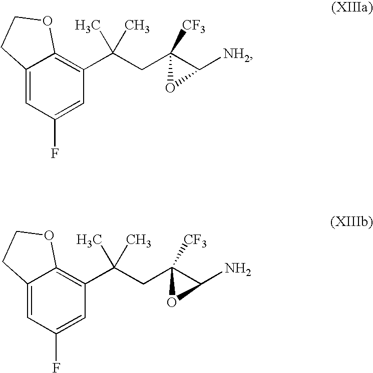
(c) carrying out the step of (i) reducing the chiral primary epoxyamine having Formula XIIIa or XIIIb to form a chiral hydroxyamine having Formula XIVa or XIVb; 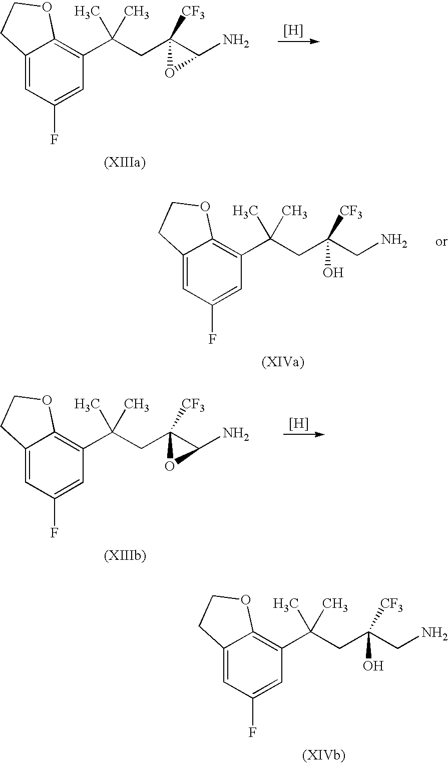
or (ii) converting the chiral primary epoxyamine having Formula XIIIa or XIIIb, under an acidic condition, to a chiral aldehyde having Formula XVa or XVb; 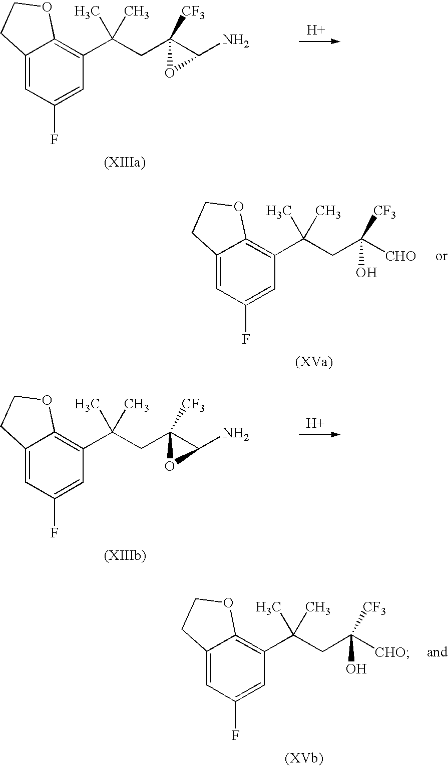
(e) reacting: (1) the chiral hydroxyamine having Formula XIVa or XIVb with a compound having a formula of 5-halo-2-methyl-quinoline under a base catalysis condition or under transition metal catalysis, when the step (d)(i) is carried out; or (2) the chiral aldehyde XVa or XVb with a compound having a formula of 5-amino-quinoline, when the step (d)(ii) is carried out, to produce the single substituted alcohol stereoisomer having Formula IIa or IIb; wherein the halo substituent at the 5 position of the substituted quinoline is selected from the group consisting of bromine, chlorine, fluorine, and iodine.

35. A method for producing a single substituted alcohol steroisomer having Formula IIIc or IIId, 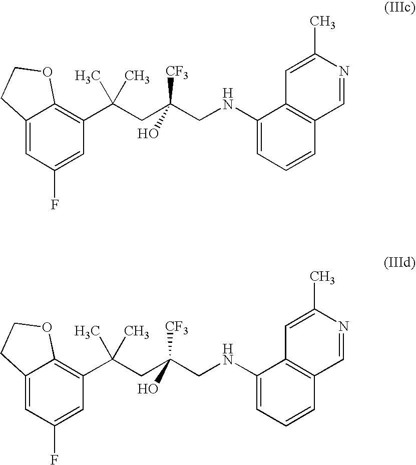
substantially free of the other stereoisomer, the method comprising:
(a) reacting a ketone having Formula X with a with an amide of an α-haloacid under a basic condition in the presence of a chiral catalyst to form a chiral epoxycarboxamide having Formula having Formula XIa or XIb; 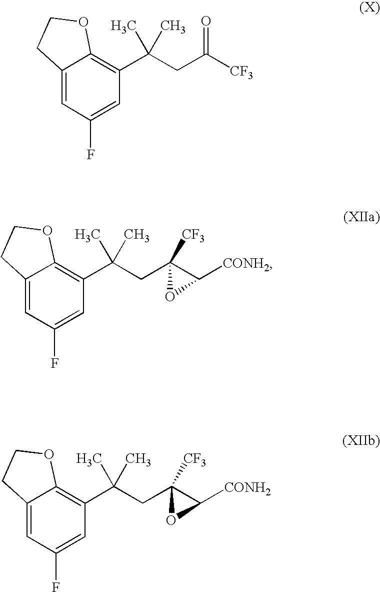
(b) converting the chiral epoxycarboxamide having Formula XIIa or XIIb to a chiral primary amine having Formula XIIIa or XIIIb under a halogen and in the presence of a base, or upon treatment with an alkali hypohalite; 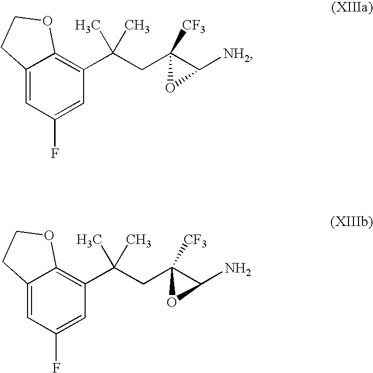
(c) carrying out the step of (i) reducing the chiral primary epoxyamine having Formula XIIIa or XIIIb to form a chiral hydroxyamine having Formula XIVa or XIVb; 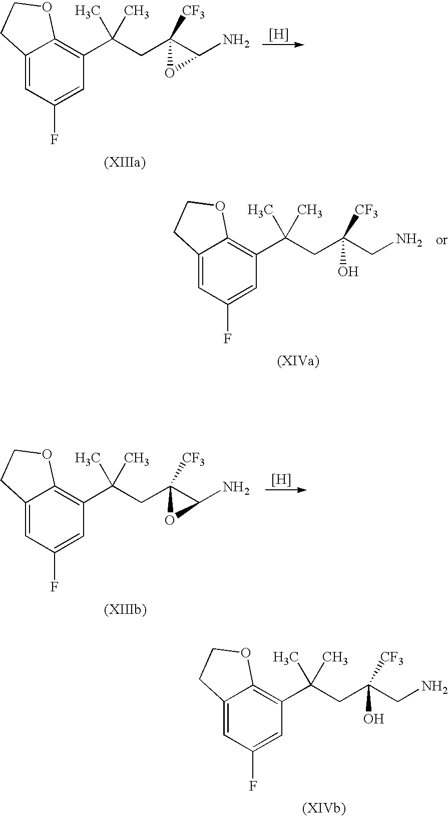
or (ii) converting the chiral primary epoxyamine having Formula XIIIa or XIIIb, under an acidic condition, to a chiral aldehyde having Formula XVa or XVb; 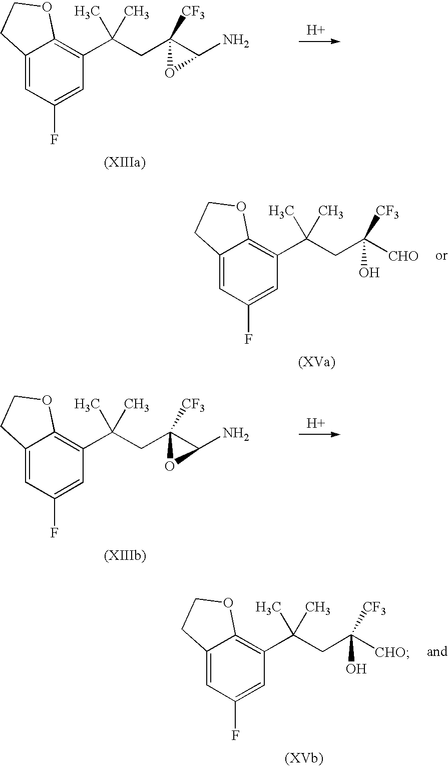
(d) reacting: (1) the chiral hydroxyamine having Formula XIVa or XIVb with a compound having a formula of 4-halo-2-methyl-isoquinoline under a base catalysis condition or under transition metal catalysis, when the step (c)(i) is carried out; or (2) the chiral aldehyde XVa or XVb with a compound having a formula of 4-amino-isoquinoline, when the step (c)(ii) is carried out, to produce the single substituted alcohol stereoisomer having Formula IIIc or IIId; wherein the halo substituent at the 4 position of the substituted isoquinoline is selected from the group consisting of bromine, chlorine, fluorine, and iodine.

36. A steroisomer of a substituted alcohol that has Formula Ia or Ib, substantially free of the other stereoisomer, produced by the method of claim 1.

37. A steroisomer of a substituted alcohol that has Formula Ia or Ib, substantially free of the other stereoisomer, produced by the method of claim 2.

38. A steroisomer of a substituted alcohol that has Formula Ia or Ib, substantially free of the other stereoisomer, produced by the method of claim 3.

39. A steroisomer of a substituted alcohol that has Formula Ia or Ib, substantially free of the other stereoisomer, produced by the method of claim 4.

40. A steroisomer of a substituted alcohol that has Formula Ia or Ib, substantially free of the other stereoisomer, produced by the method of claim 5.

41. A steroisomer of a substituted alcohol that has Formula Ia or Ib, substantially free of the other stereoisomer, produced by the method of claim 6.

42. A steroisomer of a substituted alcohol that has Formula IIa or IIb, substantially free of the other stereoisomer, produced by the method of claim 28.

43. A steroisomer of a substituted alcohol that has Formula IIc or IId, substantially free of the other stereoisomer, produced by the method of claim 29.

44. A steroisomer of a substituted alcohol that has Formula IIa or IIb, substantially free of the other stereoisomer, produced by the method of claim 30.

45. A steroisomer of a substituted alcohol that has Formula IIc or IId, substantially free of the other stereoisomer, produced by the method of claim 31.

46. A steroisomer of a substituted alcohol that has Formula IIIa or IIIb, substantially free of the other stereoisomer, produced by the method of claim 32.

47. A steroisomer of a substituted alcohol that has Formula IIIc or IIId, substantially free of the other stereoisomer, produced by the method of claim 33.

48. A steroisomer of a substituted alcohol that has Formula IIIa or IIIb, substantially free of the other stereoisomer, produced by the method of claim 34.

49. A steroisomer of a substituted alcohol that has Formula IIIc or IIId, substantially free of the other stereoisomer, produced by the method of claim 35.
